(19) United States
(12) Patent Application Publication
(10) Pub. No.: US 2012/0115235 A1
(43) Pub. Date: May 10, 2012

(54) ENHANCED CELLULASE EXPRESSION IN S. DEGRADANS

(75) Inventor: Steven W. Hutcheson, Columbia, MD (US)

(73) Assignee: UNIVERSITY OF MARYLAND, College Park, MD (US)

(21) Appl. No.: 13/262,978

(22) PCT Filed: Apr. 6, 2010
(86) PCT No.: PCT/US2010/030075

§ 371 Date: Jan. 9, 2012

Related U.S. Application Data
(60) Provisional application No. 61/166,773, filed on Apr. 6, 2009.

(51) Int. Cl. C12N 1/21; C12N 15/74

(52) U.S. Cl. 435/471; 435/252.3

(57) ABSTRACT

The invention provides organisms and methods of using and making organisms with enhanced cellulase expression.

### RELATED APPLICATIONS

This application claims priority from U.S. Provisional Patent Application No. 61/166,773 filed on Apr. 6, 2009 and incorporated, herein, by reference in its entirety.

### FIELD OF THE INVENTION

The invention is generally directed to degradative enzymes and systems. In particular, the present invention is directed to plant cell wall degrading enzymes and associated proteins found in Saccharophagus degradans, systems containing such enzymes and/or proteins, and methods of using the systems to obtain biofuels such as ethanol.

### BACKGROUND OF THE INVENTION

Cellulases and related enzymes have been utilized in food, beer, wine, animal feeds, textile production and laundering, pulp and paper industry, and agricultural industries. Various such uses are described in the paper “Cellulases and related enzymes in biotechnology” by M. K. Bhat (Biotechnical Advances 18 (2000) 355-383), the subject matter of which is hereby incorporated by reference in its entirety.

The cell walls of plants are composed of a heterogenous mixture of complex polysaccharides that interact through covalent and noncovalent means. Complex polysaccharides of higher plant cell walls include, for example, cellulose (β-1,4-glucan) which generally makes up 35-50% of carbon found in cell wall components. Cellulose polymers self associate through hydrogen bonding, van der Waals interactions and hydrophobic interactions to form semi-crystalline cellulose microfibrils. These microfibrils also include noncrystalline regions, generally known as amorphous cellulose. The cellulose microfibrils are embedded in a matrix formed of hemicelluloses (including, e.g., xylans, arabinans, and mannans), pectins (e.g., galacturonans and galactans), and various other β-1,3 and β-1,4 glucans. These matrix polymers are often substituted with, for example, arabinose, galactose and/or xylose residues to yield highly complex arabinoxylans, arabinogalactans, galactomannans, and xyloglucans. The hemicellulose matrix is, in turn, surrounded by polyphenolic lignin.

The complexity of the matrix makes it difficult to degrade by microorganisms as lignin and hemicellulose components must be degraded before enzymes can act on the core cellulose microfibrils. Ordinarily, a consortium of different microorganisms is required to degrade cell wall polymers to release the constituent monosaccharides. For saccharification of plant cell walls, the lignin must be permeabilized and hemicellulose removed to allow cellulose-degrading enzymes to act on their substrate. For industrial saccharification of cell walls, large amounts of primarily fungal cellulases are added to processed feedstock that has been treated with dilute sulfuric acid at high temperature and pressure to permeabilize the lignin and partially saccharify the hemicellulose constituents.

Saccharophagus degradans strain 2-40 (herein referred to as “S. degradans 2-40” or “2-40”) is a representative of an emerging group of marine bacteria that degrade complex polysaccharides (CP). S. degradans has been deposited at the American Type Culture Collection and bears accession number ATCC 43961. S. degradans 2-40, formerly known and referred to synonymously herein as Saccharophagus degradans strain 2-40, is a marine-proteobacterium that was isolated from decaying Sparina alterniflora, a salt marsh cord grass in the Chesapeake Bay watershed. Consistent with its isolation from decaying plant matter, S. degradans strain 2-40 is able to degrade many complex polysaccharides, including cellulose, pectin, and xylan, which are common components of the cell walls of higher plants. S. degradans strain 2-40 is also able to depolymerize algal cell wall components, such as agar, agarose, and laminarin, as well as protein, chitin, starch, pullulan, and alginic acid. In addition to degrading this plethora of polymers, S. degradans strain 2-40 can utilize each of the polysaccharides as the sole carbon source. Therefore, S. degradans strain 2-40 is not only an excellent model of microbial degradation of insoluble complex polysaccharides (ICPs) but can also be used as a paradigm for complete metabolism of these ICPs. ICPs are polymerized saccharides that are used for form and structure in animals and plants. They are insoluble in water and therefore are difficult to break down.

Saccharophagus degradans strain 2-40 requires at least 1% sea salts for growth and will tolerate salt concentrations as high as 10%. It is a highly pleomorphic, Gram-negative bacterium that is aerobic, generally rod-shaped, and motile by means of a single polar flagellum. Previous work has determined that S. degradans can degrade at least 10 different carbohydrate polymers (CP), including agar, chitin, alginic acid, carboxymethylcellulose (CMC), β-glucan, laminarin, pectin, pullulan, starch and xylan (Ensor, Stotz et al. 1999). In addition, it has been shown to synthesize a true tyrosinase (Kelley, Coyne et al. 1990). 16S rDNA analysis shows that S. degradans is a member of the gamma-subclass of the phylum Proteobacteria, related to Microbulbifer hydrolyticus (Gonzalez and Weiner 2000) and to Teridinibacter sp., (Distel, Morrill et al. 2002) cellulolytic nitrogen-fixing bacteria that are symbionts of shipworms.

The agarase, chitinase and alginase systems have been generally characterized (Ekborg et al, 2006; Howard et al 2003ab; Howard et al, 2004). Zymogram activity gels indicate that all three systems are comprised of multiple depolymerases and multiple lines of evidence suggest that at least some of these depolymerases are attached to the cell surface (Stotz 1994; Whitehead 1997; Chakravorty 1998). Activity assays reveal that the majority of S. degradans enzyme activity resides with the cell fraction during logarithmic growth on CP, while in later growth phases the bulk of the activity is found in the supernatant and cell-bound activity decreases dramatically (Stotz 1994).

The oldest methods studied to convert lignocellulosic materials to saccharides are based on acid hydrolysis (see, e.g., review by Grethlein, Chemical Breakdown Of Cellulosic Materials, J. APPL. CHEM. BIOTECHNOL. 28:296-308 (1978)). This process can involve the use of concentrated or dilute acids. For example, U.S. Pat. Nos. 5,221,537 and 5,536,325, incorporated by reference herein in their entireties, describe a two-step process for the acid hydrolysis of lignocellulosic material to glucose. These processes have numerous disadvantages including, for example, recovery of the acid, the specialized materials of construction required, the need to minimize water in the system, and the high production of degradation products which can inhibit the fermentation to ethanol.

To overcome the problems of the acid hydrolysis process, cellulose conversion processes are being developed using enzymatic hydrolysis. See, for example, U.S. Pat. No. 5,916,780, incorporated by reference herein in its entirety, which discloses enzymatic hydrolysis with a pre-treatment step to break down the integrity of the fiber structure and make the cellulose more accessible to attack by cellulase enzymes in the treatment phase.

U.S. Pat. No. 6,333,181, incorporated by reference herein in its entirety, discloses production of ethanol from lignocellulosic material by treatment of a mixture of lignocellulose, cellulose, and an ethanologenic microorganism with ultrasound.

The microbial degradation of cellulose is of interest due to applications in the sugar-dependent production of alternative biofuels (Rubin, E. M. (2008) Nature 454:841-845). Cellulose is the core polymer of plant cell walls, typically comprising 40% or more of the plant cell wall, and is organized into microfibrils formed of parallel high molecular weight β-1,4-linked D-glucose polymers. (Himmel, M. E. (2007) Science 316:982-982). As cellulose microfibrils are generally too large for cellular uptake by microorganisms, they must be depolymerized extracellularly through the action of synergistically-acting glucanases in order to metabolize the material. Secreted glucanases carry a glycoside hydrolase (GH) catalytic domain from one of several families that can be joined to one or more carbohydrate binding modules (CBMs) though flexible hydrophylic linkers. (Boraston et al. (2004) Biochem J 382:769-781). Both endo- and exo-acting glucanases can be found in some GH families. Exo-acting glucanases are processive, catalyzing multiple reactions after adsorption to the substrate, whereas endo-acting glucanases typically catalyze a single reaction prior to release but can also be processive in some cases. (Doi et al. (2004) Nat Rev Microbiol 2:541-551; Wilson, D. B. (2008) Ann NY Acad Sci 1125:289-297).

There are well-characterized cellulolytic systems of fungi and bacteria that employ multiple endo- and exo-acting glucanases in the degradation of cellulose. Microorganisms producing noncomplexed cellulase systems secrete a variety of endo-β-1,4-glucanases that release cellodextrins from crystalline or amorphous cellulose, exo-acting cellobiohydrolases specific to either the non-reducing or reducing end of cellulose or cellodextrin to generate cellobiose, and β-glucosidases to convert cellobiose to glucose. (Himmel, M. E. (2007) Science 316:982-982). For example, the wood soft rot fungus, Hypocrea jecorina, produces up to eight secreted β-1,4-endoglucanases (Cel5A, Cel5B, Cel7B, Cel12A, Cel45A, Cel61A, Cel61B, Cel74A), two cellobiohydrolases (Cel6A, Cel7A), and several β-glucosidases (e.g., Bgl3A). (Martinez, D. et al (2008) Nat Biotechnol 26:1193-1193). Another well-characterized noncomplexed cellulase system is found in Thermobifida fusca, a filamentous soil bacterium that is a major degrader of organic material found in compost piles. (Wilson, D. B. (2004) Chem Rec 4:72-82). This bacterium also secretes several endoglucanases and cellobiohydrolases specific to either the nonreducing and reducing end of cellulose. The T. fusca Cel5A, Cel6A and Cel9B are classic β-1,4-endoglucanases whereas Cel9A is a processive β-1,4-endoglucanase. (Wilson, D. B. (2004) Chem Rec 4:72-82).

An alternative mechanism to degrade cellulose is found in microorganisms producing complexed cellulolytic systems, such as those found in cellulolytic clostridia. In these microorganisms, several β-1,4-endoglucanases, including processive endoglucanases, and cellobiohydrolases assemble on surface-associated scaffoldin polypeptides to form a cellulose-degrading multiprotein complexes known as cellulosomes (Doi et al. (2004) Nat Rev Microbiol 2:541-551; Bayer et al. (1998) Curr Opin Struc Biol 8:548-557). The unifying theme in noncomplexed and complexed cellulolytic systems is the importance of cellobiohydrolases in converting cellulose and cellodextrins to soluble cellobiose. Recently a complete cellulolytic system was reported in the marine bacterium Saccharophagus degradans (Taylor, L. E. et al (2006) J Bacteriol 188:3849-3861; Weiner, R. M. et al (2008) PLOS Genet. 4:e100087). This bacterium is capable of growth on both crystalline and noncrystalline cellulose and produces multiple cellulases that can be detected in zymograms of cell lysates (Taylor, L. E. et al (2006) J Bacteriol 188:3849-3861). The genome sequence of this bacterium predicts the cellulolytic system of this bacterium consists of ten GH5-containing β-1,4-endoglucanases (Cel5A, Cel5B, Cel5C, Cel5D, Cel5E, Cel5F, Cel5G, Cel5H, Cel5I, Cel5J), two GH9 β-1,4-endoglucanases (Cel9A, Cel9B), one cellobiohydrolase (Cel6A), five β-glucosidases (Bgl1A, Bgl1B, Bgl3C, Ced3A, Ced3B) and a cellobiose phosphorylase (Cep94A) (Taylor, L. E. et al (2006) J Bacteriol 188:3849-3861; Weiner, R. M. et al (2008) PLOS Genet. 4:e100087).

The apparent absence of homologs to clostridial scaffoldins in the genome sequence and to dockerin-like domains in the cellulases suggests S. degradans 2-40 produces a noncomplexed cellulolytic system. Two unusual features of this cellulolytic system are the large number of GH5 endoglucanases and the presence of only one annotated cellobiohydrolase, Cel6A, which is postulated to act from the nonreducing end of the polymer (Taylor, L. E. et al (2006) J Bacteriol 188:3849-3861; Weiner, R. M. et al (2008) PLOS Genet. 4:e100087). This enzyme, however, is expressed at a low level (Zhang and Hutcheson, unpublished) and a homolog to a cellobiohydrolase acting from the reducing end of cellulose was not detected in this bacterium's genome (Taylor, L. E. et al (2006) J Bacteriol 188:3849-3861).

There exists a need to identify enzyme systems that use cellulose as a substrate, express the genes encoding the proteins using suitable vectors, identify and isolate the amino acid products (enzymes and non-enzymatic products), and use these products as well as organisms containing these genes for purposes, such as the production of ethanol and uses described in the Bhat paper. There is also a need in the art of using lignocellulosic materials for production of biofuels such as ethanol, to develop more effective treatment methods that result in greater yields of biofuels.

### SUMMARY OF THE INVENTION

One aspect of the present invention is directed to systems of plant wall active carbohydrases and related proteins.

The invention provides a modified bacterium in which cell wall degrading enzymes are constitutively expressed at an increased rate of expression. In more specific embodiments the modified bacterium is modified to over express one or more enzymes selected from Cel5G, Cel5H or Cel5J. The modified bacterium may also express one or more enzymes from BglA, Bgl1B, Bgl3C, Ced3A, Ced3B. The bacterium may be S. degradans.

A further aspect of the invention is directed to a method for the degradation of substances comprising cellulose. The method involves contacting the cellulose containing substances with one or more compounds obtained from Saccharophagus degradans strain 2-40.

Another aspect of the present invention is directed to groups of enzymes that catalyze reactions involving cellulose.

Another aspect of the present invention is directed to polynucleotides that encode polypeptides with cellulose degrading or cellulose binding activity.

A further aspect of the invention is directed to chimeric genes and vectors comprising genes that encode polypeptides with cellulose depolymerase activity.

A further aspect of the invention is directed to a method for the identification of a nucleotide sequence encoding a polypeptide comprising any one of the following activities from S. degradans: cellulose depolymerase, or cellulose binding. An S. degradans genomic library can be constructed in E. coli and screened for the desired activity. Transformed E. coli cells with specific activity are created and isolated.

Another aspect of the invention is directed to a method for producing ethanol or another byproduct that is the product of a fermentative process from lignocellulosic material, comprising treating lignocellulosic material with an effective saccharifying amount of one or more compounds listed in FIGS. 4-11 to obtain saccharides and converting the saccharides to produce ethanol. Conversion of sugars to ethanol or another fermentative product and recovery may be accomplished by, but are not limited to, any of the well-established methods known to those of skill in the art. For example, through the use of an ethanologenic microorganism, such as Zymomonas, Erwinia, Klebsiella, Xanthomonas, and Escherichia, preferably Escherichia coli K011 and Klebsiella oxytoca P2 or butanogenic organism such as Clostridium acetobutylicum.

A further aspect of the invention is directed to a method for producing ethanol from lignocellulosic material, comprising contacting lignocellulosic material with a microorganism expressing an effective saccharifying amount of one or more compounds listed in FIGS. 4-11 to obtain saccharides and converting the saccharides to produce ethanol. See above.

A further aspect of the invention is directed to a method for producing ethanol from lignocellulosic material, comprising contacting lignocellulosic material with an ethanologenic microorganism expressing an effective saccharifying amount of one or more compounds listed in FIGS. 4-11 to produce ethanol. Such an ethanologenic microorganism expresses an effective amount of one or more compounds listed in FIGS. 4-11 to saccharify the lignocellulosic material and an effective amount of one or more enzymes or enzyme systems which, in turn, catalyze (individually or in concert) the conversion of the saccharides to ethanol. See above.

Further aspects of the invention are directed to utilization of the cellulose degrading substances in food, beer, wine, animal feeds, textile production and laundering, pulp and paper industry, and agricultural industries.

The present invention is advantageous in that saccharification of plant cell walls and ethanol production processes including saccharification may be obtained without permeabilizing lignin and/or removing or partially saccharifying the hemicellulose or hemicellulose constituents before the cellulose-degrading enzymes can act on their substrate. The present invention also allows for saccharification and ethanol production processes including saccharification without or with a reduced amount of fungal cellulases, acids (e.g., sulfuric acid), high temperatures, and high pressures in the saccharification process.

The invention provides a method for creating a mixture of enzymes for the degradation of plant material. Preferably, this degradation occurs without chemical pretreatments of the plant material. This method comprises growing Saccharophagus degradans in the presence of a given plant material and then measuring the expression of enzymes that are expressed in the Saccharophagus degradans. The enzymes that undergo increased expression in the presence of the given plant material are combined to form a mixture of enzymes for the degradation of the given plant material.

The invention also provides a modified bacterium in which enzymes that are upregulated in Saccharophagus degradans in the presence of a given plant material are constitutively expressed at an increased rate of expression. The modified bacterium is able to degrade the given plant material at a much faster rate than a non-modified bacterium. The invention also provides a modified bacterium in which enzymes that are constituitively expressed in Saccharophagus degradans are expressed at an increased rate. The modified bacterium is able to degrade the given plant material at a much faster rate than a non-modified bacterium. For example, an enzyme that is constituitively expressed is Bgl1A.

The invention also provides a method of producing ethanol, wherein a bacterium is used to degrade one or more plant materials, and the simpler sugars that result from the degradation process are used to produce ethanol in an aqueous mixture with the one or more plant materials. The ethanol is produced by any way known in the art. In one embodiment, the ethanol is produced from the degradation product of the bacterium by a yeast cell. The bacterium may be Saccharophagus degradans strain 2-40 or it may be a modified bacterium that expresses specific cell wall degrading enzymes. In specific embodiments of the invention the aqueous mixture of bacteria and one or more plant materials comprises at least 1% salt and/or at most 10% salt. These embodiments of the invention are also used to make sugar by omitting steps to convert sugars to ethanol.

The invention also provides a method of producing ethanol, wherein a mix of enzymes is used to degrade a given plant material, and the simpler sugars that result from the degradation process are used to produce ethanol. The ethanol is produced by any way known in the art. In one embodiment, the ethanol is produced from the degradation product of the bacterium by a yeast cell. The mix of enzymes is two or more of the enzymes upregulated in Saccharophagus degradans strain 2-40 in response to the presence of the given plant material. The enzymes are harvested from the Saccharophagus degradans strain 2-40 by any method known in the art. In specific embodiments of the invention the aqueous mixture of proteins and one or more plant materials comprises at least 1% salt and/or at most 10% salt. In other specific embodiments, the Saccharophagus degradans strain 2-40 is grown until it reaches an OD600 from about 0.3 to about 0.5 on the first portion of plant material. In other specific embodiments, the Saccharophagus degradans strain 2-40 is grown until it reaches an OD600 from about 5 to about 10. In other specific embodiments, the Saccharophagus degradans strain 2-40 is grown until it reaches an OD600 greater than 10. These embodiments of the invention are also used to make sugar by not adding yeast.

In more specific embodiments of the mixes of enzymes used to degrade a given plant material, the invention includes a composition comprising Cel5H and Cel5I. In another specific embodiment, the invention includes a composition comprising Cel5H and Cel5F.

In alternative embodiments of the mixes of enzymes used to degrade a given plant material, the invention includes a composition comprising Cel5F, Cel5H, Cel5I, Cep94A and Cep94B. In more specific embodiments, the invention includes a composition further comprising Cel6A, Bgl3C and Cel9B. In more specific embodiments, the invention includes a composition further comprising Cel5A, Cel5B, Cel5C, Cel5D, Cel5E, Cel5G, Cel5J, Cel9A, Bgl1A, Bgl1B, Ced3A and Ced3B. In additional embodiment, the composition of the invention further comprises yeast.

In alternative embodiments of the mixes of enzymes used to degrade a given plant material, the invention includes a composition comprising Cel5E, Cel5I, Cel9A, Bgl1A and Ced3B. In more specific embodiments, the invention includes a composition further comprising Cel5B, Cep94A and Cep94B. In additional embodiment, the composition of the invention further comprises yeast.

In alternative embodiments of the mixes of enzymes used to degrade a given plant material, the invention includes a composition comprising Cel5F, Cel9A, Bgl1A and Ced3B. In more specific embodiments, the invention includes a composition further comprising Cel5B, Cel5E, Cel5I, Bgl1B, Bgl3C, Cep94A and Cep94B. In additional embodiment, the composition of the invention further comprises yeast.

In alternative embodiments of the mixes of enzymes used to degrade a given plant material, the invention includes a composition comprising Cel5I, Bgl1A, Bgl1B, Ced3B and Cep94B. In more specific embodiments, the invention includes a composition further comprising Cel5A, Cel5G, Cel9A, Bgl3C and Cep94A. In additional embodiment, the composition of the invention further comprises yeast.

In alternative embodiments of the mixes of enzymes used to degrade a given plant material, the invention includes a composition comprising Cel5E, Cel5F, Cel5H, Cel6A and Cel9B. In more specific embodiments, the invention includes a composition further comprising Cel5I, Bgl3C and Cep94A. In other specific embodiments, the invention includes a composition further comprising Cel5C, Cel5D and Ced3A. In additional embodiment, the composition of the invention further comprises yeast.

In alternative embodiments of the mixes of enzymes used to degrade a given plant material, the invention includes a composition comprising Xyn10a, Xyn10b and Xyn11a. In more specific embodiments, the invention includes a composition further comprising Xyn10D and Xyn11B. In additional embodiment, the composition of the invention further comprises yeast.

In another embodiment of the invention, a nucleic acid that encodes an inventive desired cellulase is provided. In another embodiment, the DNA is in a vector. In a further embodiment, the vector is used to transform a host cell.

In another embodiment of this invention, a method for producing an inventive desired cellulase is provided. The method comprises the steps of culturing a host cell transformed with a nucleic acid encoding a desired cellulase in a suitable culture medium under suitable conditions to produce the desired cellulase and obtaining the desired cellulase so produced.

### BRIEF DESCRIPTION OF THE FIGURES

FIG. 1A shows the chemical formula of cellulose.

FIG. 1B illustrates the physical structure of cellulose.

FIG. 2A illustrates the degradation of cellulose fibrils.

FIG. 2B shows the chemical representation of cellulose degradation to cellobiose and glucose.

FIG. 3 shows SDS-PAGE and Zymogram analysis of S. degradans culture supernatants.

FIG. 4 lists the predicted cellulases of S. degradans 2-40 (the sequences from FIGS. 4-10 are disclosed as SEQ ID NOs 1-214, respectively in order of appearance, 1—Acronyms, cel=cellulase, ced=cellodextrinase, bql=6-glucosidase, cep=cellobiose/cellodextrin phosphorylase; 2—Protein identified by tandem mass spectrometry in supernatant concentrates. Growth substrates: av=avicel, aq=agarose, al=alginate, cm=CMC, xn=xylan; 3—MW and amino acid count calculated using the protParam (protein parameters) tool at the Expasy website based on the DOE/JGI gene model amino acid sequence translations and includes any predicted signal peptide; 4—Predictions of function and GH, GT and CBM module determination according to CAZy ModO analysis by B. Henrissat, AFMB-CRNS; Da^{gg}ers (t) indicate lack of a secretion signal sequence; 5—Nonstandard module abbreviations, LPB=lipobox motif, PSL=polyserine linker, EPR=glutamic acid-proline rich region, PLP=phospholipase-like domain, number in parentheses indicates the length of the indicated feature in amino acid residues; 6—Refseq accession number of gene amino acid sequence from the Entrez protein database.

FIG. 5 lists the predicted xylanases, xylosidases and related accessories of S. degradans 2-40.

FIG. 6 lists the predicted pectinases and related accessories of S. degradans 2-40, 1—Acronyms, pet=pectate lyase, pes=pectin methylesterase, rql=rhamnogalacturonan lyase; 2—MW and amino acid count calculated using the protParam (protein parameters) tool at the Expasy website based on the DOE/JGI gene model amino acid sequence translations and includes any predicted signal peptide; 3—Predictions of function and GH, GT, PL, CE and CBM module determination according to CAZy ModO analysis by B. Henrissat, AFMB-CRNS; 4—Module abbreviations, CE=carbohydrate esterase, FN3=fibronectin type3-like domain, LPB=lipobox motif, PL=pectate lyase, PSR=polyserine region, EPR=glutamic acid-proline rich region, number in parentheses indicates the length of the indicated feature in amino acid residues; 5—Refseq accession number of gene model amino acid sequence from the Entrez Pubmed database;

FIG. 7 lists the arabinanases and arabinogalactanases of S. degradans 2-40.

FIG. 8 lists the mannanases of S. degradans 2-40.

FIG. 9 lists the laminarinases of S. Degradans 2-40, Superscripts: 1—Acronyms, lam=laminarinase; 2—MW and amino acid count calculated using the protParam (protein parameters) tool at the Expasy website based on the DOE/JGI gene model amino acid sequence translations and includes any predicted signal peptide; 3-Predictions of function and GH, GT, PL, CE and CBM module determination according to CAZy Mod( ) analysis by B. Henrissat, AFMB-CRNS; 4—Module abbreviations: TSP3=thrombospondin type3 repeats, COG3488=thiol-oxidoreductase like domain of unknown function (Interestingly, a similar domain is found in cbm32A: see table 7), PSD=polyserine domain, TMR=predicted transmembrane region, FN3=fibronectin type3like domain, EPR=glutamic acid-proline rich region, CADG=cadherin-like calcium binding motif, number in parentheses indicates the length of the indicated feature in amino acid residues; 5—Refseq accession number can be used to retrieve the gene model amino acid sequence from the Entrez Pubmed database.

FIG. 10 lists selected carbohydrate-binding module proteins of S. degradans 2-40

FIG. 11 lists the recombinant proteins of S. degradans 2-40 and a comparison of predicted vs. observed molecular weights thereof.

FIG. 12 provides a zymogram of S. degradans GH5 Cellulase activities. After fractionation by SDS-PAGE and renaturing, retained substrate was stained using Congo Red. Zones of clearing represent glucanase activity. Similar results were obtained in zymograms containing HE-cellulose. With the exception of Cel5E and Cel5J, expression of each protein in the Rossetta 2™ (DE3) host was equivalent in commassie blue-stained gels. To resolve the activity of polypeptides in the zymograms, the samples were diluted. The amount of protein in each well was equivalent to the original cell culture (nanoliters): Cel5A (100), Cel5B (1000), Cel5C (7000), Cel5D (7000), Cel5E (1000), Cel5F (100), Cel5G (1), Cel5H (10), Cel5I (7000), Cel5J (10). Precision Plus molecular weight markers (Bio-Rad, Hercules, Calif.) were used as molecular weight markers.

FIG. 13 provides an analysis of products formed by S. degradans Cel5H activity. Left: Products formed on the indicated substrate: A, Avicel™; FP, filter paper; and SC, phosphoric acid swollen cellulose. Reaction mixtures containing purified Cel5H and the substrate were incubated at 50° C. for 16 h. Two μl aliquots were spotted on Silica G plates and resolved using a nitromethane-propanol-water solvent system. The markers, G1-G4, represent glucose, cellobiose, cellotriose and cellotetraose. Right: Time course of products released by the activity of S. degradans Cel5H on swollen cellulose. Reaction conditions were as described in the Materials and Methods and products resolved as above. Time is indicated in minutes.

FIG. 14 relates to the processivity of S. degradans Cel5H and Cel5H′. Purified Cel5H or Cel5H′ were incubated with filter paper for the indicated time and products formed as reducing sugar determined. Diamonds (top line) indicate soluble reducing sugar detected (μmol cellobiose). Squares (bottom line) indicate insoluble reducing sugar (μmol glucose). Linear trendlines were calculated using y=mx. For release of soluble reducing sugar, m=0.0108 with a goodness of fit R²=0.97). For formation of insoluble reducing sugar, m=0.0020. R²=0.94.

FIG. 15 relates to the effect of S. degradans Cel5H, T. fusca Cel9B and T. fusca Cel6B on the viscosity of carboxymethyl cellulose. The viscosity was measured as a function of time as described in the Materials and Methods. The viscosity is given in centipoise (cP).

FIG. 16 relates to the phylogenetic analysis of S. degradans GH5 domains and their processivity on filter paper. The sequences of the GH5 domains found in the S. degradans cellulases identified by Taylor et al. (J Bacteriol 188:3849-3861 (2006)) together with the closest homologs identified in another organism by BLAST were extracted using the SMART algorithm and subjected to nearest neighbor analysis as described in the Materials and Methods. The resulting phylogenetic tree as well as the structures predicted by SMART are shown. The processivities of the indicated enzymes were determined as in FIG. 3. Gene designations with accession number of the closest homolog: Cel5A-N-YP_{—}435061 (Endoglucanase [Hahella chejuensis KCTC 2396]); Cel5A-C-YP_{—}528706 (Sde 3237, Cel5H); Cel5B-ZP_{—}00510594 (Glycoside hydrolase, family 5: Clostridium cellulosome enzyme, dockerin type I: Carbohydrate binding domain, family 11 [Clostridium thermocellum ATCC 27405]); cel5C ZP_{—}01246425 (Glycoside hydrolase, family 5 [Flavobacterium johnsoniae UW101]); Cel5D ZP_{—}01115721 (Endoglucanase family 5 [Reinekea sp. MED297]); Cel5E (Sde 2490, Cel5B); Cel5F ABA02176 (cellulase [uncultured bacterium])—new best hit(7/8/8): Cellvibrio japonicus Ueda107; Cel5G YP_{—}528706 (Sde 3237, Cel5H); Cel5H YP_{—}528708 (Sde_{—}3239, Cel5G); Cel5I ZP_{—}01113981 (endo-1,4-β-glucanase [Reinekea sp. MED297]); Cel5J YP_{—}435061 (Endoglucanase [Hahella chejuensis KCTC 2396]).

FIG. 17 relates to the analysis of products formed by digestions of secreted S. degradans 2-40 enzymes. S. degradans 2-40 was grown on Avicel™ for 24 hours and supernatant decanted. The supernatant was then digested with swollen cellulose (SC) for 24 hours. Two μl aliquots were spotted onto the TLC plate as described in the materials and methods. The result for the digestion of swollen cellulose is compared to the enzyme supernatant and the SC substrate prior to digestion.

FIG. 18 provides an illustration of one embodiment of the method for producing a new S. degradans strain.

FIG. 19 provides a restriction map of the pDSK600 plasmid.

FIG. 20 provides a zymogram of Zym5 and Zym8 grown on Avicel™. Lane 1 represents original wild-type bacterium; lane 2 represents Zym5 (strain over-expressing Cel5G); lane 3 represents Wild-type bacterium again; and lane 4 represents Zym8 strain (strain over expressing Cel5J).

FIG. 21 provides a growth curve of modified host cells (Zym strains) grown on glucose.

FIG. 22 provides a graph showing corrected data showing real distribution of the cellulase activity between cells and the medium. The total activity produced by each strain in this assay are (units Azcl Cellulose Degradation).

### DETAILED DESCRIPTION

The invention provides genetic modification of marine bacteria with processive cellulases for the more efficient saccharification of cellulose.

Saccharophagus degradans 2-40 is a marine bacterium capable of degrading all of the polymers found in the higher plant cell wall using secreted and surface-associated enzymes. This bacterium has the unusual ability to saccharify whole plant material without chemical pretreatments. For example, this bacterium is able to utilize as sole carbon sources (generation time at same concentration, h): glucose (2.6), Avicel™ (2.25), oat spelt xylan (1.6), newsprint (>6), whole and pulverized corn leaves (>6), and pulverized Panicum vigatum leaves (>6), indicating the production of synergistically-acting hemicellulases, pectinases, cellulases, and possibly ligninases.

Analysis of the genome sequence of S. degradans 2-40 reveals an abundance of genes coding for enzymes that are predicted to degrade plant-derived carbohydrates. To date, S. degradans is the only sequenced marine bacterium with apparently complete cellulase and xylanase systems, as well as a number of other systems containing plant-wall active carbohydrases.

Thus it appears that S. degradans can play a significant role in the marine carbon cycle, functioning as a “super-degrader” that mediates the breakdown of CP from various algal, plantal, and invertebrate sources. The remarkable enzymatic diversity, novel surface features (ES), and the apparent localization of carbohydrases to ES make S. degradans 2-40 an intriguing organism in which to study the cell biology of CP metabolism and surface enzyme attachment.

It has now been discovered that S. degradans has a complete complement of enzymes, suitably positioned, to degrade plant cell walls. This has been accomplished by the following approaches: a) annotation and genomic analysis of S. degradans plant-wall active enzyme systems, b) identification of enzymes and other proteins which contain domains or motifs that may be involved in surface enzyme display, c) the development of testable models based on identified protein motifs, and d) cloning and expression of selected proteins for the production of antibody probes to allow testing of proposed models of surface enzyme display using immunoelectron microscopy.

These efforts have been greatly facilitated by the recent sequencing of the genome of S. degradans strain 2-40, allowing a strategy where genes which code for proteins with potential involvement in surface attachment may be identified based on sequence homology with modules or domains known to function in surface attachment and/or adhesion.

Enzymatic and non-enzymatic ORFs with compelling sequence elements are identified using BLAST and other amino acid sequence alignment and analysis tools. Genes of interest can be cloned into E coli, expressed with in-frame polyhistidine affinity tag fusions and purified by nickel ion chromatography, thus providing the means of identifying and producing recombinant S. degradans proteins for study and antibody probe production.

The genome sequence of S. degradans was recently obtained in conjunction with the Department of Energy's Joint Genome Initiative (JGI). The finished draft sequence dated Jan. 19, 2005 comprises 5.1 Mbp contained in a single contiguous sequence. Automated annotation of open reading frames (ORFs) was performed by the computational genomics division of the Oak Ridge National Laboratory (ORNL), and the annotated sequence is available on the World Wide Web (http://genome.ornl.gov/microbial/mdeg).

The initial genome annotation has revealed a variety of carbohydrases, including a number of agarases, alginases and chitinases. Remarkably, the genome also contains an abundance of enzymes with predicted roles in the degradation of plant cell wall polymers, including a number of ORFs with homology to cellulases, xylanases, pectinases, and other glucanases and glucosidases. In all, over 180 open reading frames with a probable role in carbohydrate catabolism were identified in the draft genome.

To begin to define the cellulase, xylanase and pectinase systems of S. degradans, genes were initially classified as belonging to one of those systems by BLAST homology. Ambiguous ORFs were tentatively assigned to the class of the best known hit. Other tools used to refine this tentative classification include Pfam (Protein families database of alignments and HMMs; http://www.sanger.ac.uk/Software/Pfam/) and SMART (Simple Modular Architecture Research Tool; http://smart.embl-heidelberg.de/) which use multiple alignments and hidden Markov models (statistical models of sequence consensus homology) to identify discreet modular domains within a protein sequence. These analyses were relatively successful; however, a number of ORFs remained difficult to classify based on sequence homology alone.

Enzymes have traditionally been classified by substrate specificity and reaction products. In the pre-genomic era, function was regarded as the most amenable (and perhaps most useful) basis for comparing enzymes and assays for various enzymatic activities have been well-developed for many years, resulting in the familiar EC classification scheme. Cellulases and other O-Glycosyl hydrolases, which act upon glycosidic bonds between two carbohydrate moieties (or a carbohydrate and non-carbohydrate moiety—as occurs in nitrophenol-glycoside derivatives) are designated as EC 3.2.1.-, with the final number indicating the exact type of bond cleaved. According to this scheme an endo-acting cellulase (1,4-β-endoglucanase) is designated EC 3.2.1.4.

With the advent of widespread genome sequencing projects and the ease of determining the nucleotide sequence of cloned genes, ever-increasing amounts of sequence data have facilitated analyses and comparison of related genes and proteins on an unprecedented scale. This is particularly true for carbohydrases; it has become clear that classification of such enzymes according to reaction specificity, as is seen in the E.C. nomenclature scheme, is limited by the inability to convey sequence similarity. Additionally, a growing number of carbohydrases have been crystallized and their 3-D structures solved.

One of the major revelations of carbohydrase sequence and structure analyses is that there are discreet families of enzymes with related sequence, which contain conserved three-dimensional folds that can be predicted based on their amino acid sequence. Further, it has been shown that enzymes with the same three-dimensional fold exhibit the same stereospecificity of hydrolysis, even when they catalyze different reactions (Henrissat, Teeri et al. 1998; Coutinho and Henrissat 1999).

These findings form the basis of a sequence-based classification of carbohydrase modules which is available in the form of an internet database, the Carbohydrate-Active enZYme server (CAZy), at http://afmb.cnrs-mrs.fr/CAZY/index.html (Coutinho and Henrissat 1999; Coutinho and Henrissat 1999).

CAZy defines four major classes of carbohydrases, based on the type of reaction catalyzed: Glycosyl Hydrolases (GH's), Glycosyltransferases (GT's), Polysaccharide Lyases (PL's), and Carbohydrate Esterases (CE's). GH's cleave glycosidic bonds through hydrolysis. This class includes many familiar polysaccharidases such as cellulases, xylanases, and agarases. GT's generally function in polysaccharide synthesis, catalyzing the formation of new glycosidic bonds through the transfer of a sugar molecule from an activated carrier molecule, such as uridine diphosphate (UDP), to an acceptor molecule. While GT's often function in biosynthesis, there are examples where the mechanism is exploited for bond cleavage, as occurs in the phosphorolytic cleavage of cellobiose and cellodextrins. PL's use a β-elimination mechanism to mediate bond cleavage and are commonly involved in alginate and pectin depolymerization. CE's generally act as deacetylases on O- or N-substituted polysaccharides. Common examples include xylan and chitin deacetylases. Sequence-based families are designated by number within each class, as is seen with GH5: glycosyl hydrolase family 5. Members of GH5 hydrolyze β-1,4 bonds in a retaining fashion, using a double-displacement mechanism which results in retention of the original bond stereospecificity. Retention or inversion of anomeric configuration is a general characteristic of a given GH family (Henrissat and Bairoch 1993; Coutinho and Henrissat 1999). Many examples of endocellulases, xylanases and mannanases belonging to GH5 have been reported, illustrating the variety of substrate specificity possible within a GH family. Also, GH5s are predominantly endohydrolases—cleaving chains of their respective substrates at random locations internal to the polymer chains. While true for GH5, this generalization does not hold for many other GH families. In addition to carbohydrases, the CAZy server defines numerous families of Carbohydrate Binding Modules (CBM). As with catalytic modules, CBM families are designated based on amino acid sequence similarity and conserved three-dimensional folds.

The CAZyme structural families have been incorporated into a new classification and nomenclature scheme, developed by Bernard Henrissat and colleagues (Henrissat, Teeri et al. 1998). Traditional gene/protein nomenclature assigns an acronym indicating general function and order of discovery; in this scheme an organism's cellulase genes are designated celA, celB, etc., regardless of their actual mechanism of action on cellulose. Some researchers have attempted to convey more information by naming cellulases as endoglucanases (engA, engB) or cellobiohydrolases (cbhA, cbhB), however this requires determination of function in vitro and still fails to convey relatedness of protein sequence and structure. CAZyme nomenclature retains the familiar acronym to indicate the functional system a gene belongs to and incorporates the family number designation. Capital letters after the family number indicate the order of report within a given organism system. An example is provided by two endoglucanases, CenA and CenB, of Cellulomonas fimi. In the old nomenclature nothing can be deduced from the names except order of discovery. Naming them Cel6A and Cel9A, respectively, makes it immediately clear that these two cellulases are unrelated in sequence, and so belong to different GH families (where Cel stands for cellulase, and 9 for glycosyl hydrolase family nine). While this scheme does not distinguish between endo- and exo-activity, these designations are not absolute and can be included in discussion of an enzyme when relevant (i.e. the cellobiohydrolase Cel6A, the endoxylanase Xyn10B). Catalytic modules take precedence in naming carbohydrases; since many (or even most) carbohydrases contain at least one CBM, they are named for their enzymatic module. If more than one catalytic domain is present, they are named in order from N-terminus to C-terminus, i.e. cel9A-cel48A contains a GH9 at the amino-terminus and a GH48 at the carboxy-terminus. Both domains act against cellulose. There are, however, many examples of CBM modules occurring on proteins with no predicted carbohydrase module. In the absence of some other predicted functional domain (like a protease) these proteins are named for the CBM module family. If there are multiple CBM families present, then naming is again from amino to carboxy end, i.e. cbm2D-cbm10A. This nomenclature has been widely accepted and will be used in the naming of all S. degradans plant-wall active carbohydrases and related proteins considered as part of this study.

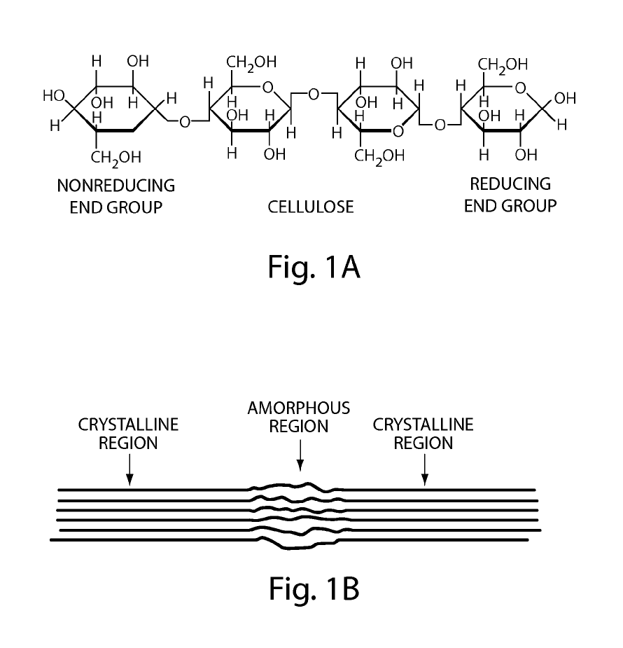

The cell walls of higher plants are comprised of a variety of carbohydrate polymer (CP) components. These CP interact through covalent and non-covalent means, providing the structural integrity plants required to form rigid cell walls and resist turgor pressure. The major CP found in plants is cellulose, which forms the structural backbone of the cell wall. See FIG. 1A. During cellulose biosynthesis, chains of poly-β-1,4-D-glucose self associate through hydrogen bonding and hydrophobic interactions to form cellulose microfibrils which further self-associate to form larger fibrils. Cellulose microfibrils are somewhat irregular and contain regions of varying crystallinity. The degree of crystallinity of cellulose fibrils depends on how tightly ordered the hydrogen bonding is between its component cellulose chains. Areas with less-ordered bonding, and therefore more accessible glucose chains, are referred to as amorphous regions (FIG. 1B). The relative crystallinity and fibril diameter are characteristic of the biological source of the cellulose. The irregularity of cellulose fibrils results in a great variety of altered bond angles and steric effects which hinder enzymatic access and subsequent degradation.

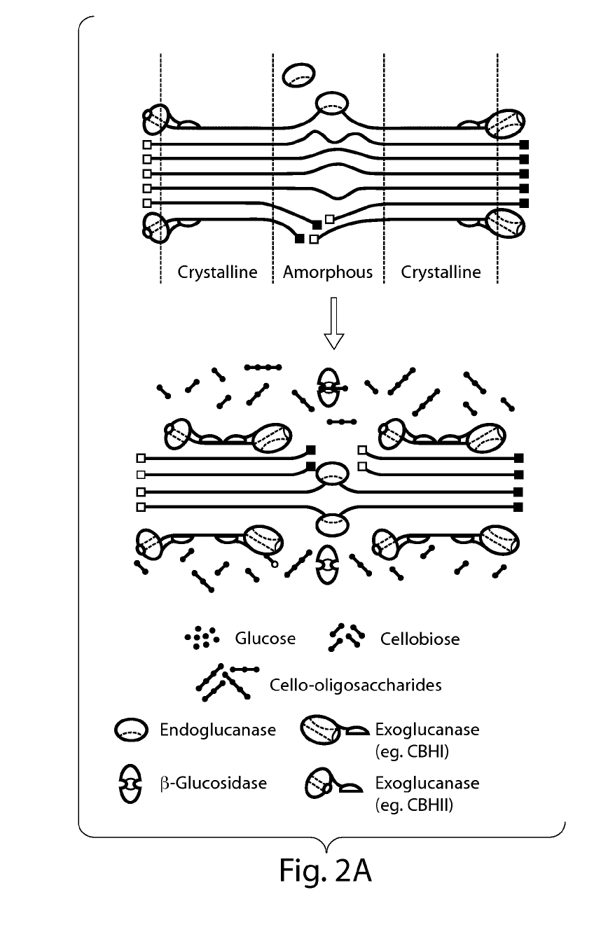

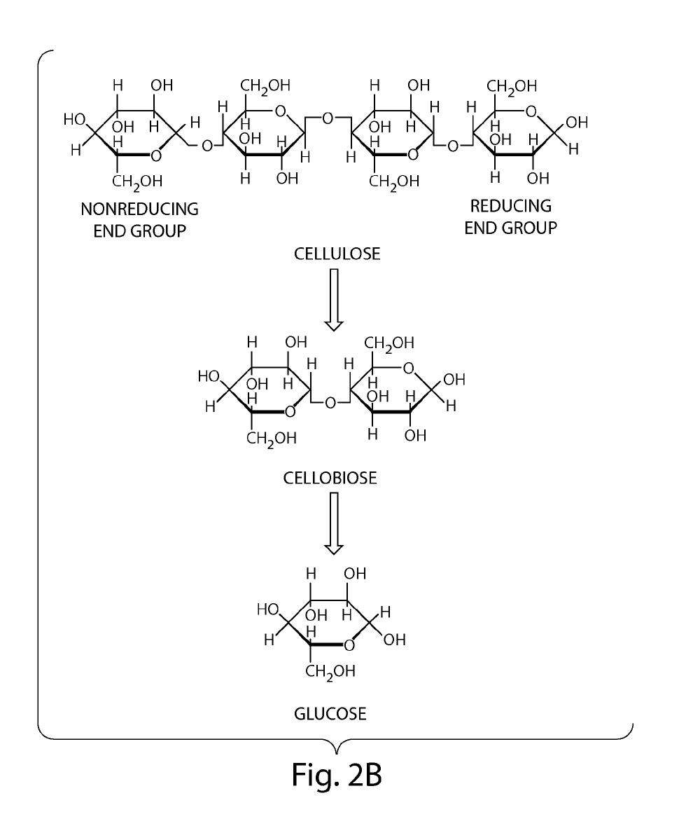

The general model for cellulose depolymerization to glucose involves a minimum of three distinct enzymatic activities (See FIGS. 2A and 2B). Endoglucanases cleave cellulose chains internally to generate shorter chains and increase the number of accessible ends, which are acted upon by exoglucanases. These exoglucanases are specific for either reducing ends or non-reducing ends and frequently liberate cellobiose, the dimer of cellulose (cellobiohydrolases). The accumulating cellobiose is cleaved to glucose by cellobiases (β-1,4-glucosidases). In many systems an additional type of enzyme is present: cellodextrinases are β-1,4-glucosidases which cleave glucose monomers from cellulose oligomers, but not from cellobiose. Because of the variable crystallinity and structural complexity of cellulose, and the enzymatic activities required for is degradation, organisms with “complete” cellulase systems synthesize a variety of endo and/or exo-acting β-1,4-glucanases.

For example, Cellulomonas fimi and Thermobifida fusca have each been shown to synthesize six cellulases while Clostridium thermocellum has as many as 15 or more. Presumably, the variations in the shape of the substrate-binding pockets and/or active sites of these numerous cellulases facilitate complete cellulose degradation. Organisms with complete cellulase systems are believed to be capable of efficiently using plant biomass as a carbon and energy source while mediating cellulose degradation. The ecological and evolutionary role of incomplete cellulose systems is less clear, although it is believed that many of these function as members of consortia (such as ruminal communities) which may collectively achieve total or near-total cellulose hydrolysis.

In the plant cell wall, microfibrils of cellulose are embedded in a matrix of hemicelluloses (including xylans, arabinans and mannans), pectins (galacturonans and galactans), and various β-1,3 and β-1,4 glucans. These matrix polymers are often substituted with arabinose, galactose and/or xylose residues, yielding arabinoxylans, galactomannans and xyloglucans—to name a few. The complexity and sheer number of different glycosyl bonds presented by these non-cellulosic CP requires specific enzyme systems which often rival cellulase systems in enzyme count and complexity. Because of its heterogeneity, plant cell wall degradation often requires consortia of microorganisms.

Objectives—S. degradans synthesize complete multi-enzyme systems that degrade the major structural polymers of plant cell walls. A) define cellulase and xylanase systems, determining the activities of genes for which function cannot be predicted by sequence homology; and B) genomic identification and annotation of other plant-degrading enzyme systems by sequence homology (i.e. pectinases, laminarinases, etc.).

From the ORNL annotation it is clear that the S. degradans genome contains numerous enzymes with predicted activity against plant cell wall polymers. This is particularly surprising since S. degradans is an estuarine bacterium with several complex enzyme systems that degrade common marine polysaccharides such as agar, alginate, and chitin. Defining multienzyme systems based on automated annotations is complicated by the presence of poorly conserved domains and/or novel combinations of domains. There are many examples of this in the plant-wall active enzymes of S. degradans. Accordingly, the ORNL annotations of carbohydrase ORFs were manually reviewed with emphasis on the modular composition and then assigned to general groups based on the substrate they were likely to be involved with (i.e. cellulose or xylan degradation). These genomic sequence analyses resulted in a pool of about 25 potential cellulases, 11 xylanases and 17 pectinases.

When sequence homology is well-conserved, highly accurate predictions of function are possible. Therefore, to verify the presence of functioning cellulase and xylanase systems in S. degradans, zymograms and enzyme activity assays were performed as discussed below. Also, attempts were made to identify enzymes from S. degradans culture supernatants using Mass Spectrometry based proteomics.

Next, more sophisticated genomic analyses were used to predict function where possible and to identify ORFs which require functional characterization to determine their roles, if any, in the cellulase and xylanase systems. ORFs which belong to other plant wall-active enzyme systems were tentatively classified based on the sequence analyses and functional predictions of B. Henrissat.

To gain insight into the induction and expression of S. degradans cellulases and xylanases, specific activities were determined for Avicel™ and xylan-grown cells and supernatants by dinitrosalicylic acid reducing-sugar assays (DNSA assays), as discussed in the Experimental Protocols section at the end of this proposal. Xylanase activity was measured for Avicel™-grown cultures, and vice versa, in order to investigate possible co-induction of activity by these two substrates which occur together in the plant cell wall.

Growth on either Avicel™ or xylan yields enzymatic activity against both substrates, suggesting co-induction of the cellulase and xylanase systems. As with other S. degradans carbohydrase systems, highest levels of activity were induced by the homologous substrate. The results also reveal some key differences in the expression of these two systems. When grown on Avicel™, cellulase activity is cell-associated in early growth and accumulates significantly in late-stage supernatants. Cell and supernatant fractions exhibit low levels of xylanase activity that remain roughly equal throughout all growth phases. In contrast, xylan-grown cultures exhibit the majority of xylanase and cellulase activity in the cellular fraction throughout the growth cycle. Cellulase activity does not accumulate in the supernatant and xylanase activity accumulates modestly, but still remains below the cell-bound activity.

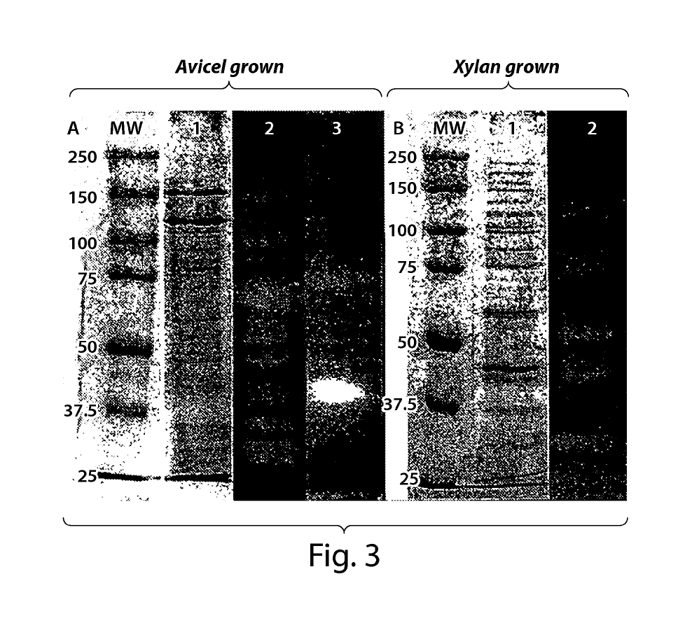

Enzyme activity gels (zymograms) of Avicel™ and xylan grown cell pellets and culture supernatants were analyzed to visualize and identify expressed cellulases and xylanases. The zymograms revealed five xylanolytic bands in xylan-grown supernatants (FIG. 3), four of which correspond well with the calculated MW of predicted xylanases (xyl/arb43G-xyn10D: 129.6 kDa, xyn10E: 75.2 kDa, xyn10C: 42.3 kDa, and xyn11A: 30.4 kDa; see Table 2). Avicel™-grown cultures showed eight active bands with MWs ranging from 30-150 kDa in CMC zymograms. CMC is generally a suitable substrate for endocellulase activity. These zymograms clearly demonstrate that S. degradans synthesizes a number of endocellulases of varied size during growth on Avicel™-indicative of a functioning multienzyme cellulase system. Together, the CMC and xylan zymograms confirm the results of the genomic analyses and the inducible expression of multienzyme cellulase and xylanase systems in S. degradans 2-40.

The amino acid translations of all gene models in the S. degradans draft genome were analyzed on the CAZy ModO (Carbohydrase Active enzyme Modular Organization) server at AFMB-CRNS. This analysis identified all gene models that contain a catalytic module (GH, GT, PL, or CE) and/or a CBM. In all, the genome contains 222 gene models containing CAZy domains, most of which have modular architecture. Of these, 117 contain a GH module, 39 have GTs, 29 PLs, and 17 CE. Many of these carry one or more CBM from various families. There are also 20 proteins that contain a CBM but no predicted carbohydrase domain.

Detailed comparisons of S. degradans module sequences to those in the ModO database allowed specific predictions of function for modules where the sequence of the active site is highly conserved. For example, Cel9B (from the gel slice MS/MS) contains a GH9 module which is predicted to function as an endocellulase, a CBM2 and a CBM10 module.

When catalytic module sequences are less conserved, only a general mechanism can be predicted. This is the case with gly5M which contains a GH5 predicted to be either a 1, 3 or 1,4 glucanase—sequence analysis cannot be certain which, and so the acronym designation “gly” for glycanase.

The results of this detailed evaluation and analysis were used to assign genes to cellulase, xylanase, pectinase, laminarinase, arabinanase and mannanase systems. Each system was also assigned the relevant accessory enzymes, i.e. cellobiases belong to the cellulase system and xylosidases belong to the xylanase system. Genes with less-conserved GH modules which have the most potential to function as cellulases, xylanases or accessories were identified and designated as needing demonstration of function.

The major criteria for assigning function will be the substrate acted upon, and the type of activity detected. As such, the various enzyme activity assays will focus on providing a qualitative demonstration of function rather than on rigorously quantifying relative activity levels. The assays required are dictated by the substrate being tested, and are discussed in more detail in Experimental Protocols. For cellulose it is important to distinguish between β-1,4-endoglucanase (endocellulase), β1,4-exoglucanase (cellobiohydrolase), and β-1,4-glucosidase (cellobiase) activities. This will be accomplished using zymograms to assay for endocellulase, DNSA reducing-sugar assays for cellobiohydrolase, and p-nitrophenol-β-1,4-cellobioside (pnp-cellobiose) for cellobiase activity. The combined results from all three assays will allow definition of function as follows: a positive zymogram indicates endocellulase activity, a negative zymogram combined with a positive DNSA assay and a negative pnp-cellobiose assay indicates an exocellulase, while a negative zymogram and DNSA with a positive pnp-cellobiose result will imply that the enzyme is a cellobiase. To date the predicted biochemical activities as an endoglucanase have been demonstrated for Cel5A, Cel5B, Cel5E, Cel5F, Cel5G, Cel5H, Cel5J, Cel9A, and Cel9B. Cellobiohydrolase activity has been shown for Cel6A and β-glucosidase activity confirmed for Bgl1A, Bgl1B, Bgl3C, Ced3A, and Ced3B. Cep94A has been shown to be a cellobiose phosphorylase.

Xylanase (β-1,4-xylanase), laminarinase (β-1,3-glucanase), and mixed glucanase (β-1,3(4)-glucanase) activity will be determined by xylan, laminarin and barley glucan zymograms, respectively. Unlike cellulose, there do not appear to be any reports of “xylobiohydrolases” or other exo-acting enzymes which specifically cleave dimers from these substrates. Thus zymograms will suffice for demonstrating depolymerase (endo) activity and pnp-derivatives will detect monosaccharide (exo) cleavage. The pnp-derivatives used in this study will include pnp-α-L-arabinofuranoside, -α-L-arabinopyranoside, -β-L-arabinopyranoside, -β-D-cellobioside, -α-D-xylopyranoside and -β-D-xylopyranoside. These substrates were chosen based on the possible activities of the domains in question. The assays will allow determination of function for any α- and β-arabinosidases, β-cellobiases, β-xylosidases, bifunctional α-arabinosidase/β-xylosidases, and α-xylosidases—which cleave α-linked xylose substituents from xyloglucans. The pnp-derivative assays will be run in 96-well microtiter plates using a standard curve of p-nitrophenol concentrations, as discussed in Experimental Protocols. To date Xyn11A and Xyn11B have been shown to have xylanase activity.

### Experimental Protocols

### Zymograms

All activity gels were prepared as standard SDS-PAGE gels with the appropriate CP substrate incorporated directly into the separating gel. Zymograms are cast with 8% polyacrylamide concentration and the substrate dissolved in dH₂O and/or gel buffer solution to give a final concentration of 0.1% (HE-cellulose), 0.15% (barley β-glucan), or 0.2% (xylan). Gels are run under discontinuous conditions according to the procedure of Laemmli (Laemmli 1970) with the exception of an 8 minute treatment at 95° C. in sample buffer containing a final concentration of 2% SDS and 100 mM dithiothreitol (DTT). After electrophoresis, gels are incubated at room temperature for 1 hour in 80 ml of a renaturing buffer of 20 mM PIPES buffer pH 6.8 which contains 2.5% Triton X-100, 2 mM DTT and 2.5 mM CaCl₂. The calcium was included to assist the refolding of potential calcium-binding domains such as the tsp3s of Lam16A.

After the 1 hour equilibration, gels were placed in a fresh 80 ml portion of renaturing buffer and held overnight at 4° C. with gentle rocking. The next morning gels were equilibrated in 80 ml of 20 mM PIPES pH6.8 for 1 hour at room temperature, transferred to a clean container, covered with the minimal amount of PIPES buffer and incubated at 37° C. for 4 hours. Following incubation gels were stained for 30 minutes with a solution of either 0.25% Congo red in dH₂O (HE-cellulose, β-glucan and xylan) or 0.01% Toluidine blue in 7% acetic acid. Gels were destained with 1M NaCl for Congo red and dH₂O for Toluidine blue until clear bands were visible against a stained background.

### DNS Reducing-Sugar Assays

Saccharifying enzyme activity is assayed using DNS assay for reducing sugars (Ghose 1987. Pure Apl Chem 59; 257-268). Test substrates include avicel, CMC, phosphoric-acid swollen cellulose (PASC), Barley glucan, laminarin, and xylan dissolved at 1% in 20 mM PIPES pH 6.8 (Barley glucan and laminarin, 0.5%). Barley glucan, laminarin and xylan assays are incubated 2 hours at 50° C.; avicel, CMC and PASC assays were incubated 1 hour at 37° C. Samples are assayed in triplicate, corrected for blank values, and levels estimated from a standard curve. Enzymatic activity is calculated, with one unit (U) defined as 1 μM of reducing sugar released/minute and reported as specific activity in U/mg protein.

Exoglycosidase Activity Assays: pnp-Derivatives

Purified proteins were assayed for activity against pNp derivatives of α-L-arabinofuranoside, -α-L-arabinopyranoside, -β-L-arabinopyranoside, -β-D-cellobioside, -α-D-glucopyranoside, -β-D-glucopyranoside, -α-D-xylopyranoside and -β-D-xylopyranoside. 25 μl of enzyme solution was added to 125 μl of 5 mM substrate solution in 20 mM PIPES pH 6.8, incubated for 30 min at 37° C., and A₄₀₅ was determined. After correcting for blank reactions, readings were compared to a p-nitrophenol standard curve and reported as specific activities in U/mg protein, with one unit (U) defined as 1 μmol p-Np/min.

Cloning and Expression of Saccharophagus Degradans Proteins or Saccharophagus Degradans-like proteins in E coli

The basic cloning and expression system uses pET28B (Novagen) as the vector, E coli DH5α (Invitrogen) as the cloning strain, and E coli BL-21(DE3) Rosetta2™ (DE3) cells (Novagen) for protein expression strain. This system allows the cloning of toxic or otherwise difficult genes because the vector places expression under the control of a T7 lac promoter—which is lacking in the cloning strain DH5α, thereby abolishing even low-level expression during plasmid screening and propagation. After the blue/white screen, plasmids are purified from DH5α and transformed into the expression host (Tuners). The Tuner strain has the T7 lac promoter, allowing IPTG-inducible expression of the vector-coded protein and lacks the Lon and Omp proteases.

The nucleotide sequences of gene models were obtained from the DOE JGI's Saccharophagus degradans genome web server and entered into the PrimerQuest™ design tool provided on Integrated DNA Technologies web page located at http://biotools.idtdna.com/Primerguest/. The design parameters were Optimum Tₘ 60° C., Optimum Primer Size 20 nt, Optimum GC %=50, and the product size ranges were chosen so that the primers were selected within the first and last 100 nucleotides of each ORF in order to clone as much of the gene as reasonably possible. The cloning and expression vector, pETBlue2, provides a C-terminal 6× Histidine fusion as well as the start and stop codon for protein expression. Thus, careful attention to the frame of the vector and insert sequences is required when adding 5′ restriction sites to the PCR primers. The resulting “tailed primers” were between 26 to 30 nt long, and their sequences were verified by “virtual cloning” analysis using the PDRAW software package. This program allows vector and insert DNA sequences to be cut with standard restriction enzymes and ligated together. The amino acid translations of the resulting sequences were examined to detect any frame shifts introduced by errors in primer design. Following this verification, the primers were purchased from Invitrogen (Frederick, Md.).

PCR reactions contained 10 pMol of forward and reverse primers, 1 μl of 10 mM DNTPs, 1.5 μl of 100 mM MgCl₂, and 1 μl Proof Pro® Pfu Polymerase in a 501 reaction with 0.5 μl of S. degradans genomic DNA as the template. PCRs conditions used standard parameters for tailed primers and Pfu DNA polymerase. PCR products were cleaned up with the QIAGEN QIAquick PCR Cleanup kit and viewed in 0.8% agarose gels. Following cleanup and confirmation of size, PCR products and pETBlue2 are digested with appropriate restriction enzymes, usually Ascl and Clal at 37° C. for 1 to 4 hours, cleaned up using the QIAquick kit, and visualized in agarose gels. Clean digestions are ligated using T4 DNA ligase for at least 2 hours in the dark at room temperature. Ligations are then transformed into E coli DH5α by electroporation. Transformants are incubated one hour at 37° C. in non-selective media, and then plated onto LB agar containing ampicillin and X-gal. As pETBlue2 carries an Amp^{r} gene and inserts are cloned into the lacZ ORF, white colonies contain the insert sequence. White colonies are picked with toothpicks and patched onto a new LB/Amp/X-gal plate, with three of the patched colonies also being used to inoculate 3 ml overnight broths. Plasmids are prepped from broths which correspond to patched colonies which remained white after overnight outgrowth. These plasmid preps are then singly digested with an appropriate restriction enzyme and visualized by agarose electrophoresis for size confirmation.

The plasmids are then heat-shock transformed into the Rosetta® strain. The Transformants are incubated 1 hour at 37° C. in non-selective rescue medium, plated on LB agar with Amp nd incubated overnight at 37° C. Any colonies thus selected should contain the vector and insert. This is confirmed by patching three colonies onto a Tuner medium plate and inoculating corresponding 3 ml overnight broths. The next morning the broths are used to inoculate 25 ml broths which are grown to an OD₆₀₀ of around 0.6 (2-3 hours). At this point a 1 ml aliquot is removed from the culture, pelleted and resuspended in 1/10 volume 1×SDS-PAGE treatment buffer. This pre-induced sample is frozen at −20° C. for later use in western blots. The remaining broth is then amended to 1 mM IPTG and incubated 4 hours at 37° C. Induced pellet samples are collected at hourly intervals. These samples and the pre-induced control are run in standard SDS-PAGE gels and electroblotted onto PVDF membrane. The membranes are then processed as western blots using a 1/5000 dilution of monoclonal mouse α-HisTag® primary antibodies followed by HRP-conjugated goat α-mouse IgG secondary antibodies. Bands are visualized colorimetrically using BioRad's Opti-4CN substrate kit. Presence of His tagged bands in the induced samples, but not in uninduced controls, confirms successful expression and comparison of bands from the hourly time points are used to optimize induction parameters in later, larger-scale purifications.

### Production and Purification of Recombinant Proteins

Expression strains are grown to an OD₆₀₀ of 0.6 to 0.8 in 500 ml or 1 liter broths of tuner medium.

At this point a non-induced sample is collected and the remaining culture induced by addition of 100 mM IPTG to a final concentration of 1 mM. Induction is carried out for four hours at 37° C. or for 16 hours at 25° C. Culture pellets are harvested and frozen overnight at −20° C. for storage and to aid cell lysis. Pellets are then thawed on ice for 10 minutes and transferred to pre-weighed falcon tubes and weighed. The cells are then rocked for 1 hour at 25° C. in 4 ml of lysis buffer (8M Urea, 100 mM NaH₂PO₄, 25 mM Tris, pH 8.0) per gram wet pellet weight. The lysates are centrifuged for 30 minutes at 15,000 g to pellet cell debris. The cleared lysate (supernatant) is pipetted into a clean falcon tube, where 1 ml of QIAGEN 50% Nickel-NTA resin is added for each 4 ml cleared lysate. This mixture is gently agitated for 1 hour at room temperature to facilitate binding between the Ni²⁺ ions on the resin and the His tags of the recombinant protein. After binding, the slurry is loaded into a disposable mini column and the flow thru (depleted lysate) is collected and saved for later evaluation. The resin is washed twice with lysis buffer that has been adjusted to pH 7.0; the volume of each of these washes is equal to the original volume of cleared lysate. The flow thru of these two washes is also saved for later analysis in western blots to evaluate purification efficiency.

At this point the columns contain relatively purified recombinant proteins which are immobilized by the His tags at their C-terminus. This is an ideal situation for refolding, so the column is moved to a 4° C. room and a series of renaturation buffers with decreasing urea concentrations are passed through the column. The renaturation buffers contain varying amounts of urea in 25 mM Tris pH 7.4, 500 mM NaCl, and 20% glycerol. This buffer is prepared as stock solutions containing 6M, 4M, 2M and 1M urea. Aliquots of these can be easily mixed to obtain 5M and 3M urea concentrations thus providing a descending series of urea concentrations in 1M steps. One volume (the original lysate volume) of 6M buffer is passed through the column, followed by one volume of 5M buffer, continuing on to the 1M buffer—which is repeated once to ensure equilibration of the column at 1M urea. At this point the refolded proteins are eluted in 8 fractions of 1/10^{th} original volume using 1M urea, 25 mM Tris pH 7.4, 500 mM NaCl, 20% glycerol containing 250 mM imidazole. The imidazole disrupts the Nickel ion-His tag interaction, thereby releasing the protein from the column.

Western blots are used to evaluate the amount of His tagged protein in the depleted lysate, the two washes, and the eluted fractions. If there is an abundance of recombinant protein in the depleted lysate and/or washes it is possible to repeat the process and “scavenge” more protein. Eluate fractions that contain the protein of interest are pooled and then concentrated and exchanged into storage buffer (20 mM Tris pH 7.4, 10 mM NaCl, 10% glycerol) using centricon centrifugal ultrafiltration devices (Millipore). The enzyme preparations are then aliquoted and frozen at −80° C. for use in activity assays.

In various embodiments of this invention, the cellulose degrading enzymes, related proteins and systems containing thereof, of this invention, for example including one or more enzymes or cellulose-binding proteins, have a number of uses. Many possible uses of the cellulases of the present invention are the same as described for other cellulases in the paper “Cellulases and related enzymes in biotechnology” by M. K. Bhat (Biotechnical Advances 18 (2000) 355-383), the subject matter of which is hereby incorporated by reference in its entirety. For examples, the cellulases and systems thereof of this invention can be utilized in food, beer, wine, animal feeds, textile production and laundering, pulp and paper industry, and agricultural industries.

In one embodiment, these systems can be used to degrade cellulose to produce short chain peptides for use in medicine.

In other embodiments, these systems are used to break down cellulose in the extraction and/or clarification of fruit and vegetable juices, in the production and preservation of fruit nectars and purees, in altering the texture, flavor and other sensory properties of food, in the extraction of olive oil, in improving the quality of bakery products, in brewing beer and making wine, in preparing monogastic and ruminant feeds, in textile and laundry technologies including “fading” denim material, defibrillation of lyocell, washing garments and the like, preparing paper and pulp products, and in agricultural uses.

In some embodiments of this invention, cellulose may be used to absorb environmental pollutants and waste spills. The cellulose may then be degraded by the cellulase degrading systems of the present invention. Bacteria that can metabolize environmental pollutants and can degrade cellulose may be used in bioreactors that degrade toxic materials. Such a bioreactor would be advantageous since there would be no need to add additional nutrients to maintain the bacteria—they would use cellulose as a carbon source.

In some embodiments of this invention, cellulose degrading enzyme systems can be supplied in dry form, in buffers, as pastes, paints, micelles, etc. Cellulose degrading enzyme systems can also comprise additional components such as metal ions, chelators, detergents, organic ions, inorganic ions, additional proteins such as biotin and albumin.

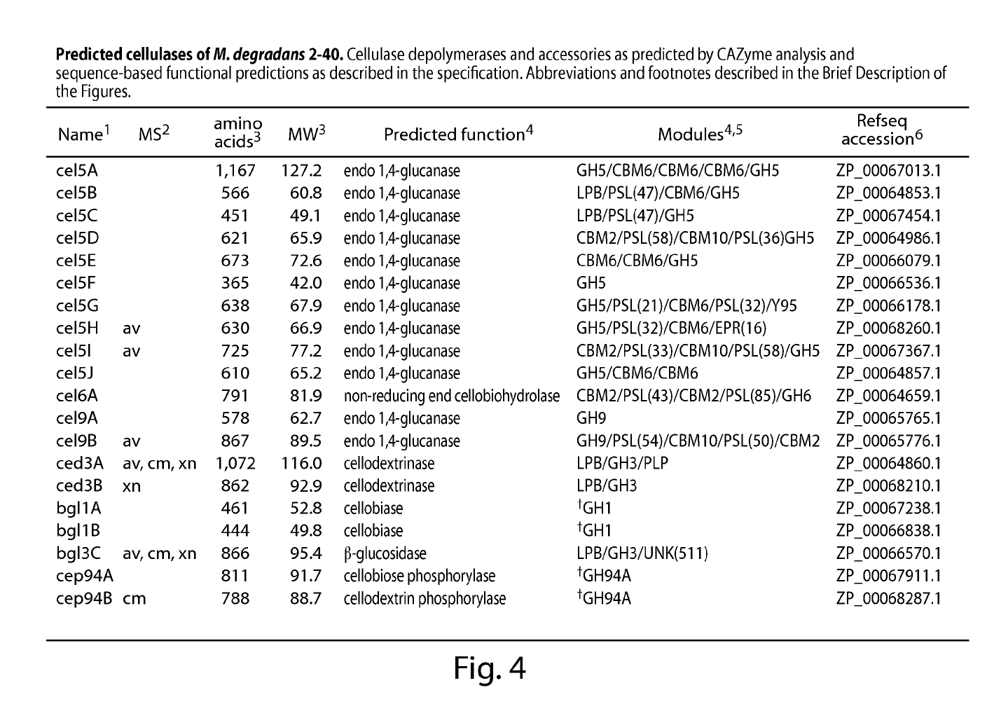

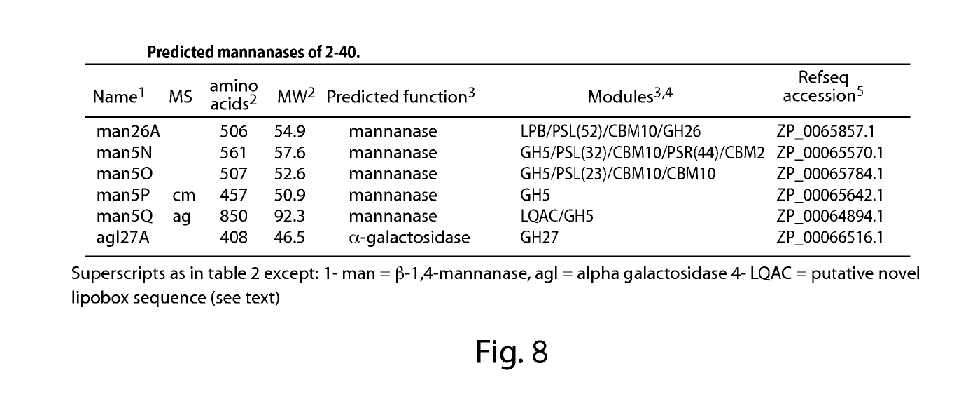

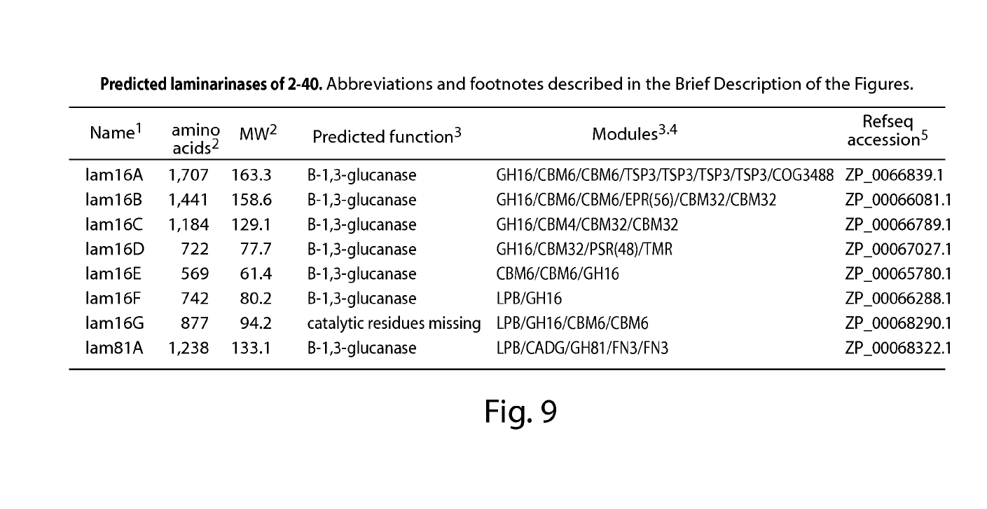

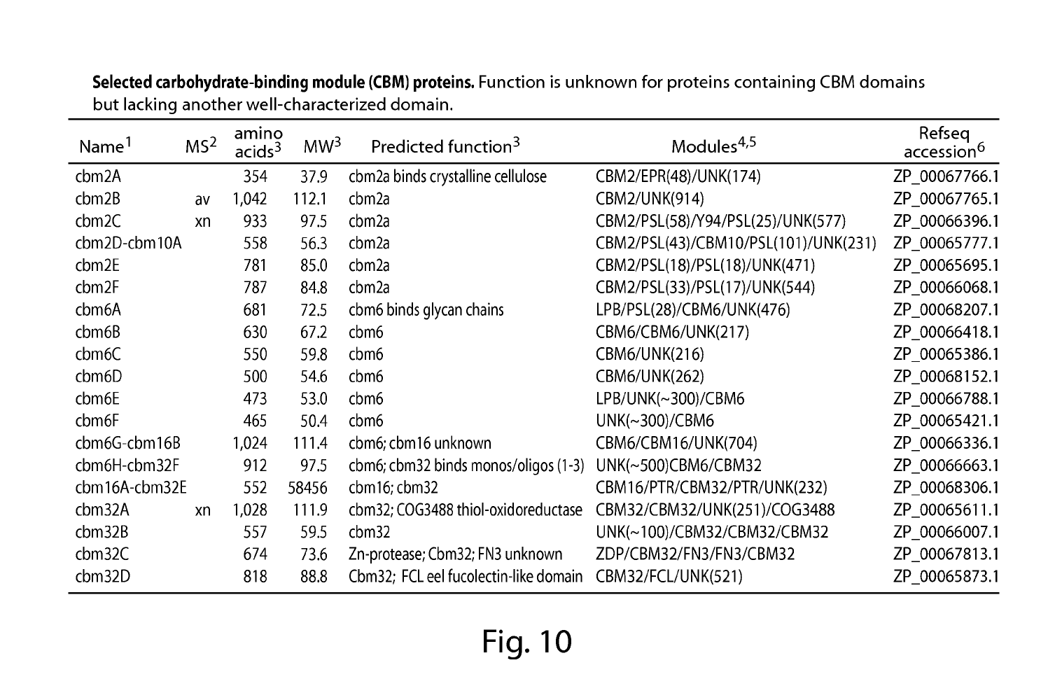

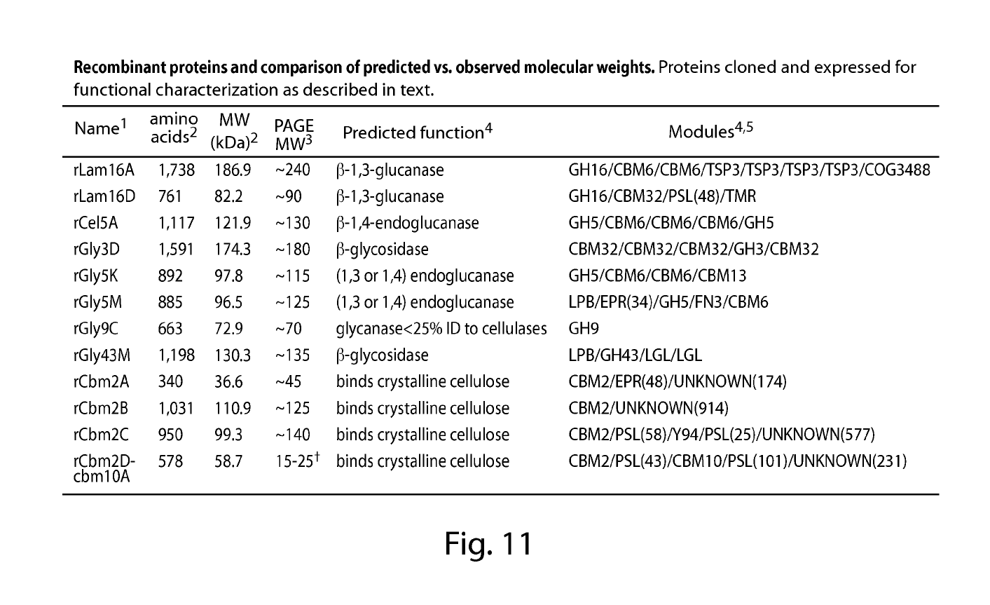

In some embodiments of this invention, the cellulose degrading systems of this invention could be applied directly to the cellulose material. For example, a system containing one, some or all of the compounds listed in FIGS. 4-11 could be directly applied to a plant or other cellulose containing item such that the system would degrade the plant or other cellulose containing item. As another example, S. degradans could be grown on the plant or other cellulose containing item, which would allow the S. degradans to produce the compounds listed in FIGS. 4-11 in order to degrade the cellulose containing item as the S. degradans grows. An advantage of using the S. degradans or systems of this invention is that the degradation of the cellulose containing plant or item can be conducted in a marine environment, for example under water.

It is one aspect of the present invention to provide a nucleotide sequence that has a homology selected from 100%, 99%, 98%, 97%, 96%, 95%, 90%, 85%, 80%, or 75% to any of the sequences of the compounds listed in FIGS. 4-11

The present invention also covers replacement of between 1 and 20 nucleotides of any of the sequences of the compounds listed in FIGS. 4-11 with non-natural or non-standard nucleotides for example phosphorothioate, deoxyinosine, deoxyuridine, isocytosine, isoguanosine, ribonucleic acids including 2-O-methyl, and replacement of the phosphodiester backbone with, for example, alkyl chains, aryl groups, and protein nucleic acid (PNA).

It is another aspect of some embodiments of this invention to provide a nucleotide sequence that hybridizes to any one of the sequences of the compounds listed in FIGS. 4-11 under stringency condition of 1×SSC, 2×SSC, 3×SSC1, 4×SSC, 5×SSC, 6×SSC, 7×SSC, 8×SSC, 9×SSC, or 10×SSC.

The scope of this invention covers natural and non-natural alleles of any one of the sequences of the compounds listed in FIGS. 4-11. In some embodiments of this invention, alleles of any one of any one of the sequences of the compounds listed in FIGS. 4-11 can comprise replacement of one, two, three, four, or five naturally occurring amino acids with similarly charged, shaped, sized, or situated amino acids (conservative substitutions). The present invention also covers non-natural or non-standard amino acids for example selenocysteine, pyrrolysine, 4-hydroxyproline, 5-hydroxylysine, phosphoserine, phosphotyrosine, and the D-isomers of the 20 standard amino acids.

Some embodiments of this invention are directed to a method for producing ethanol from lignocellulosic material, comprising treating lignocellulosic material with an effective saccharifying amount of one or more compounds listed in FIGS. 4-11, preferably cellulase cel5A listed in FIG. 4, to obtain saccharides and converting the saccharides to produce ethanol. The treating may be conducted in a marine environment, such as under water. The one or more compounds listed in FIGS. 4-11 may be present in dry form, in a buffer, or in the form of a paste, paint, or micelle.

Conversion of sugars to ethanol and recovery may be accomplished by, but are not limited to, any of the well-established methods known to those of skill in the art. For example, through the use of an ethanologenic microorganism, such as Zymomonas, Erwinia, Klebsiella, Xanthomonas, and Escherichia, preferably Escherichia coli K011 and Klebsiella oxytoca P2.

In further aspects of the present invention, the lignocellulosic material is treated with an effective saccharifying amount of all of the compounds listed in FIGS. 4-11.

In further aspects of the present invention, the one or more compounds listed in FIGS. 4-11 are from Saccharophagus degradans 2-40.

In further aspects of the present invention, the one or more compounds listed in FIGS. 4-11 are in a system consisting essentially of one or more compounds listed in FIGS. 4-11 or a system further comprising metal ions, chelators, detergents, organic ions, inorganic ions, or one or more additional proteins, such as biotin and/or albumin.

Some embodiments of this invention are directed to ethanol produced by treating lignocellulosic material with an effective saccharifying amount of one or more compounds listed in FIGS. 4-11 to obtain saccharides and converting the saccharides to produce ethanol. Conversion of sugars to ethanol and recovery may be accomplished by, but are not limited to, any of the well-established methods known to those of skill in the art. For example, through the use of an ethanologenic microorganism, such as Zymomonas, Erwinia, Klebsiella, Xanthomonas, and Escherichia, preferably Escherichia coli K011 and Klebsiella oxytoca P2.

Further embodiments of this invention are directed to a method for producing ethanol from lignocellulosic material, comprising contacting lignocellulosic material with a microorganism expressing an effective saccharifying amount of one or more compounds listed in FIGS. 4-11, preferably cellulase cel5A listed in FIG. 4, to obtain saccharides and converting the saccharides to produce ethanol. The contacting may be conducted in a marine environment, such as under water. The microorganism may be Saccharophagus degradans 2-40 or a recombinant microorganism containing a chimeric gene comprising at least one polynucleotide encoding a polypeptide comprising an amino acid sequence of at least one of the compounds listed in FIGS. 4-11; wherein the gene is operably linked to regulatory sequences that allow expression of the amino acid sequence by the microorganism. The recombinant microorganism, may be a bacteria or yeast, such as Escherichia coli. In some aspects of the present invention, the recombinant microorganism is an ethanologenic microorganism, such as microorganisms from the species Zymomonas, Erwinia, Klebsiella, Xanthomonas, and Escherichia, preferably Escherichia coli K011 and Klebsiella oxytoca P2.

Further aspects of the present invention are directed to ethanol produced by contacting lignocellulosic material with a microorganism expressing an effective saccharifying amount of one or more compounds listed in FIGS. 4-11 to obtain saccharides and converting the saccharides to produce ethanol.

A further aspect of the invention is directed to a method for producing ethanol from lignocellulosic material, comprising contacting lignocellulosic material with an ethanologenic microorganism expressing an effective saccharifying amount of one or more compounds listed in FIGS. 4-11 to produce ethanol. The ethanologenic microorganism expresses an effective amount of one or more compounds listed in FIGS. 4-11 to saccharify the lignocellulosic material and an effective amount of one or more enzymes or enzyme systems which, in turn, catalyze (individually or in concert) the conversion of the saccharides (e.g., sugars such as xylose and/or glucose) to ethanol. The one or more enzymes or enzyme systems of the ethanologenic organism may be expressed naturally or by, but not limited to, any of the methods known to those of skill in the art. For example, release of the one or more enzymes or enzyme systems may be obtained through the use of ultrasound. In some aspects of the present invention, the ethanologenic microorganism is transformed in order to be able to express one or more of the compounds listed in FIGS. 4-11. In some aspects of the present invention, the ethanologenic microorganism is from the species Zymomonas, Erwinia, Klebsiella, Xanthomonas, and Escherichia, preferably Escherichia coli K011 and Klebsiella oxytoca P2.

It is to be understood that while the invention has been described above using specific embodiments, the description and examples are intended to illustrate the structural and functional principles of the present invention and are not intended to limit the scope of the invention. On the contrary, the present invention is intended to encompass all modifications, alterations, and substitutions within the spirit and scope of the appended claims.

Analysis of the genome sequence predicts this bacterium produces at least 12 endoglucanases, 1 cellobiohydrolase, 2 cellodextrinases, 3 cellobiases, 7 xylanases, 10 “arabinases”, 5 mannases, and 14 pectinases. Analysis of zymograms and proteomic analyses of cultures revealed subsets of these enzymes are induced during growth on each of the aforementioned substrates. Induction of specific enzymes was assessed by qRT-PCR. Nomenclature for specific enzymes is explained in further detail in U.S. application Ser. No. 11/121,154, filed on May 4, 2005 and published as U.S. Publication No. 2006/0105914, which is incorporated herein in its entirety.

S. degradans effectively degrades plant material and therefore products that are constructed of plant material. Thus, the induction of the mixture of enzymes expressed in S. degradans upon exposure to a particular plant material shows that the individual enzymes and the mixture of enzymes are effective in the degradation of the plant material that the S. degradans is exposed to. This means that any two or more enzymes with increased or maintained high expression in S. degradans in response to exposure to a given plant material may be used to form an enzyme mixture for the degradation of that plant material. Two types of plant material that S. degradans is effective in degrading to simple sugars are plant material rich in cellulose and hemicellulose. Enzyme systems for degrading these two types of carbohydrates are described in greater detail below.

### Cellulose

S. degradans 2-40 expresses many enzymes for the degradation of cellulose to simple sugars. For example, in the presence of corn leaves, the celluloytic enzymes shown in Table 1, below were increased.

TABLE 1
Predicted cellulases and accessory enzymes of S. degradans
strain 2-40 and evidence supporting their identification.

| Name | Predicted function | Module(s) | MM(kDa) |
|---|---|---|---|
| Cel5A | Endo-1,4-β-glucanase (EC 3.2.1.4) | GH5/CBM6/CBM6/CBM6/GH5 | 127.2 |
| Cel5B | Endo-1,4-β-glucanase | LPB/PSL(47)/CBM6/GH5 | 60.8 |
| Cel5C | Endo-1,4-β-glucanase | LPB/PSL(47)/GH5 | 49.1 |
| Cel5D | Endo-1,4-β-glucanase | CBM2/PSL(58)/CBM10/PSL(36)/GH5 | 65.9 |
| Cel5E | Endo-1,4-β-glucanase | CBM6/CBM6/GH5 | 72.6 |
| Cel5F | Endo-1,4-β-glucanase | GH5 | 42.0 |
| Cel5G | Endo-1,4-β-glucanase | GH5/PSL(21)/CBM6/PSL(32)/Y95 | 67.9 |
| Cel5H | Endo-1,4-β-glucanase | GH5/PSL(32)/CBM6/EPR(16) | 66.9 |
| Cel5I | Endo-1,4-β-glucanase | CBM2/PSL(33)/CBM10/PSL(58)/GH5 | 77.2 |
| Cel5J | Endo-1,4-β-glucanase | GH5/CBM6/CBM6 | 65.2 |
| Cel6A | Cellobiohydrolase (EC 3.2.1.91) | CBM2/PSL(43)/CBM2/PSL(85)/GH6 | 81.9 |
| Cel9A | Endo-1,4-β-glucanase | GH9 | 62.7 |
| Cel9B | Endo-1,4-β-glucanase | GH9/PSL(54)/CBM10/PSL(50)/CBM2 | 89.5 |
| Ced3A | Cellodextrinase (EC 3.2.1.74) | LPB/GH3/PLP | 116.0 |
| Ced3B | Cellodextrinase | LPB/GH3 | 92.9 |
| Bgl1A | Cellobiase (EC 3.2.1.21) | GH1 | 52.8 |
| Bgl1B | Cellobiase | GH1 | 49.8 |
| Bgl3C | Cellobiase | LPB/GH3/UNK(511) | 95.4 |
| Cep94A | Cellobiose phosphorylase (EC 2.4.1.20) | GH94 | 91.7 |
| Cep94B | Cellodextrin phosphorylase (EC 2.4.1.49) | GH94 | 88.7 |

Enzymes that are increased in expression by S. degradans in the presence of corn leaves are likely necessary for the digestion of corn leaves to sugar. Thus, the enzymes that were increased over 20 fold, i.e. cel5F, cel5H are effective in degradation of corn leaves. Further, a mixture of all or any smaller number of the cellulolytic enzymes shown in Table 1, combined in proportion or inverse proportion to the increase of expression shown in Table 1, or the relative expression shown in FIG. 3, are used to make an enzyme mix effective for the degradation of corn leaves. Corresponding mixes are made for glucose, newsprint or Avicel™, using the information shown in Table 1. Further, mixtures for other plant materials are made through the exposure of S. degradans to the plant material and detection of the expression of degradation enzymes through detecting the RNA, protein or activity levels of the degradation enzymes.

Moreover, the following enzymes were induced as shown below in Table 2 in S. degradans when exposed to Avicel™, microcrystalline cellulose in a chemically pure form, for 10 hours.

TABLE 2
Fold increase in enzymes after 10 hours growth of
S. degradans strain 2-40 on Avicel ™.

Fold Increase after 10 h Growth on Avicel

| Basal | Low | Medium | High |
|---|---|---|---|
| Expression | (<5) | (5-25) | (>25) |
| Low (<1% GK) | | cel5C | cel5E |
| | | cel5D | cel5F |
| | | ced3A | cel5H |
| | | | cel6A |
| | | | cel9B |
| Medium (2-10%) | cel5A | cel5B | cel5I |
| | cel5G | cel5J | bgl3C |
| | | cel9A | cep94A |
| | | ced3B | |
| High (>10%) | bgl1A | cep94B | |
| | bgl1B | | |

MAX EXPRESSION: CON 2 H 4-10 H 24 H

It appears that cel5A, cel5G, cel9A, cel5B, ced3B, bgl1A and cep94B are constituitively expressed in S. degradans 2-40. After 2 hours of growth on Avicel™, cel9A expression increases. This is followed by an increase in cel5F expression at 4 hours, and increases in cel5H and cel5I expression at 10 hours. Cel5I continues to be overexpressed even at 24 hours of culture on Avicel™

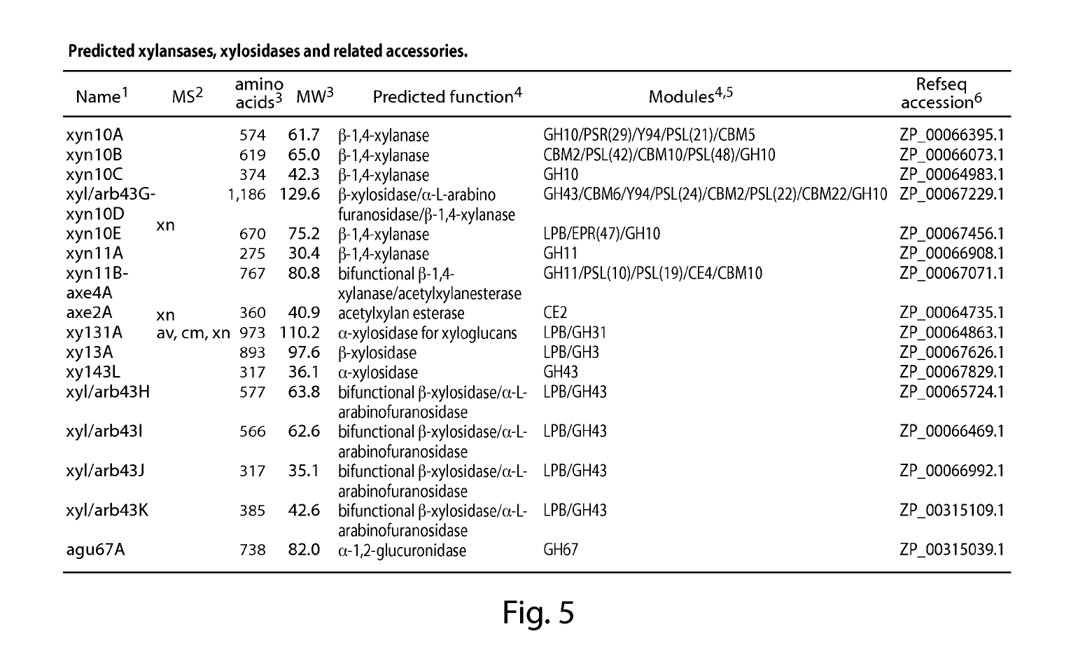

It has also been shown that Cel5I and Cel5H are particularly important for the degradation of cellulose. Cel5I is induced over 500 fold and cel5H over 100 fold when S. degradans 2-40 is exposed to cellulose (FIG. 4). Moreover, cel5H is expressed over 500 fold when S. degradans 2-40 is exposed to cellodextrins, such as cellobiose, cellotraose and cellodextrin (FIG. 5). Thus, either of these proteins could be used to efficiently break down cellulose to simple sugars.

Further, degradation enzymes with higher expression in S. degradans when exposed to a particular plant material, may be constitutively and/or over-expressed in an engineered bacterium, thus making a bacterium that is effective in the degradation of the particular plant material. For example, mixtures of proteins that are shown to be induced in S. degradans in the presence of corn leaves in Table 1, could be introduced into a bacteria so that they are constitutively expressed. These proteins could also be introduced so they are expressed at a high rate. These engineered bacteria are then used to degrade plant material, in this example, corn leaves. In one embodiment the bacterium to be engineered is S. degradans. In another embodiment, the bacterium to be engineered is E. coli.

### Hemicellulose

S. degradans 2-40 expresses many enzymes for the degradation of hemicellulose to simple sugars. Hemicellulose exists as short branched chains of sugar monomers. Sugars that make up hemicellulose include xylose, mannose, galactose, and/or arabinose. Hemicellulose forms a series of crosslinks with cellulose and pectin to form a rigid cell wall. Unlike cellulose, hemicellulose is mostly amorphous, relatively weak and susceptible to hydrolization.

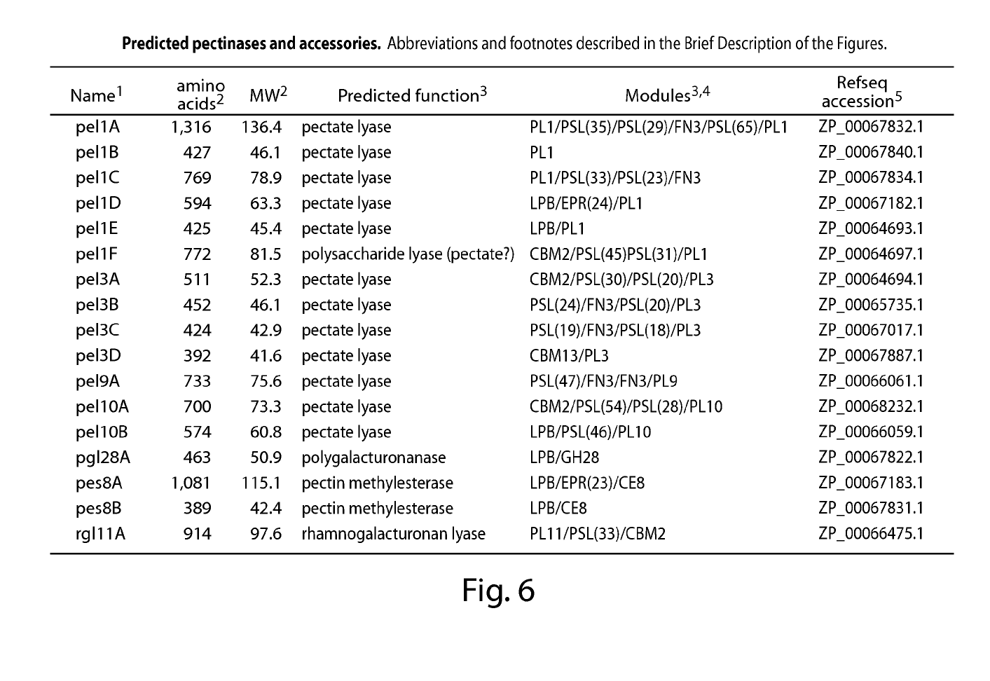

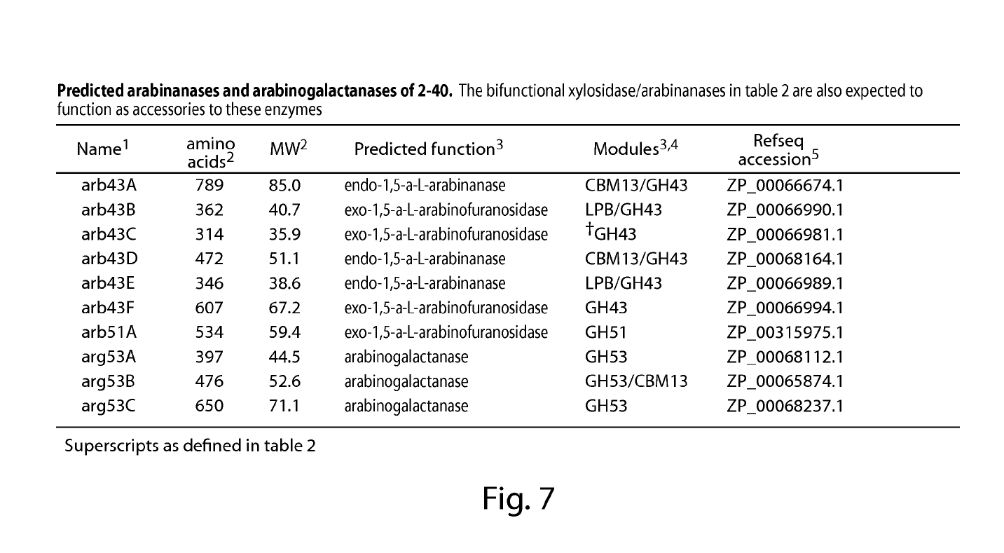

S. degradans produces many hemicellulases that are used to break down hemicellulose to simpler sugars. As shown in FIG. 6, expression of xyn10A, xyn10B, xyn10D, xyn11A and xyn11B is omduced in S. degradans 2-40 grown on xylan, containing hemicellulose. Moreover, as shown in FIG. 7, expression of xyn10A, xyn10B, xyn10D, xyn11A and xyn11B was shown after 10 hours of culture of S. degradans 2-40 on xylan. However, at 2 hours, the greatest increases in expression were for xyn11A and xyn11B, while the greatest increases in expression at 4 hours of culture of S. degradans 2-40 on xylan was xyn10A.

Thus, Xyn10a, Xyn10b, Xyn10d, Xyn11a And Xyn11b are all important for hemicellulose break down to simpler sugars. However, particular emphasis should be placed on the importance of Xyn10a, Xyn10b, Xyn11a And Xyn11b.

Enhanced Cellulase Expression in S. degradans

Bacteria are thought to degrade cellulose by using either a complexed or noncomplexed cellulolytic system composed of endoglucanases, cellobiohydrolases and β-glucosidases. The marine bacterium Saccharophagus degradans 2-40 produces a multi-component cellulolytic system that is unusual in the abundance of GH5-containing endoglucanases and β-glucosidases. Although secreted enzymes of this bacterium produce high levels of cellobiose, there is an apparent deficiency of processive enzymes, such as cellobiohydrolases. Each of the 10 annotated GH5-containing cellulases were cloned into pET28b and expressed in E. coli Rossetta2™ (DE3) to establish function. After purification to near homogeneity, all but Cel5C and Cel5I, either as the full-length polypeptide or a derivative sufficient to carry the catalytic domain, exhibited cellulase activity in zymograms consistent with their annotation as endoglucanases. One cellulase, Cel5H, showed significantly greater activity on several types of cellulose and primarily released cellobiose during digestions. The activity was processive as the ratio of soluble to insoluble products was greater than 4 irrespective of the length of digestion and resided with the catalytic domain. The processivity coupled with viscosity reduction of carboxymethyl cellulose solutions and synergisms with known cellulases indicates that Cel5H is a processive endoglucanase. Phylogenetic analyses indicated that Cel5H is a member of a separate clade of GH5-containing enzymes that also included Cel5G and Cel5J. These enzymes were also found to be processive endoglucanases whereas the other GH5 cellulases of S. degradans were classical endoglucanases forming cellodextrins. The high activity and expression of the S. degradans processive endoglucanases enables this bacterium to degrade cellulose to cellobiose independently of cellobiohydrolases.

The noncomplexed and complexed cellulolytic systems of microorganisms generally rely upon the activity of endoglucanases and cellobiohydrolases to solubilize cellulose to cellodextrins and cellobiose that are then converted to glucose or glucose 1-phosphate by the activity of β-glucosidases or cellobiose phosphorylases. Some exceptions to this model had been noted in bacterial systems that appear to lack or have deficiencies in cellobiohydrolases. (Wilson, D. B. (2008) Ann NY Acad Sci 1125:289-297). An example is the S. degradans cellulolytic system that was predicted to produce an unusual abundance of GH5 endoglucanases but has a comparative deficiency in annotated cellobiohydrolases (Taylor et al (2006) J Bacteriol 188:3849-3861). Cellobiose, however, appeared to be an early product of cellulose degradation. Thus, it was not apparent how the enzymes of the S. degradans cellulolytic system interact to solubilize and metabolize cellulose. The results presented here indicate that the S. degradans cellulolytic system utilizes a novel set of processive GH5 endoglucanases (Cel5G, Cel5H and Cel5J) to substitute for the apparent deficiency in cellobiohydrolase activity. In this model, the activity of processive endoglucanases release cellobiose from cellulose independently of classical endoglucanases. Thus the processive endoglucanases coupled with the activity of β-glucosidases or cellobiose phosphorylase are sufficient for this bacterium to metabolize cellulose.

The identification of Cel5G, Cel5H and Cel5J as processive endoglucanases acting on the β-1-4 bonds linking cellobiose units is supported by the constant ratio of soluble to insoluble products formed during reaction time courses, the release of cellobiose from pNP-cellobioside and the phylogenetic segregation of these enzymes from classic GH5 endoglucanases. Unlike classical endoglucanases that randomly cleave cellulose polymers to form a variety of degradation products, these enzymes appeared to primarily release cellobiose from a variety of cellulose substrates. Although this is not demonstrative of processivity due to the turnover rates of these enzymes (Horn, S. J. et al (2006) Proc Natl Acad Sci USA 103:18089-18094), greater than 80% of the reaction products formed by Cel5H activity were soluble, irrespective of the reaction time. Thus, S. degradans Cel5H, Cel5G and Cel5J exhibited processivity values (ratio of soluble to insoluble reaction products) in excess of 4. The processivity values reported for T. fusca Cel9A, an extensively characterized processive endoglucanase range from 3.1 to 7.0 (Li et al. (2007) Appl Environ Microbiol 73:3165-3172; Irwin et al. (1993) Biotechnol and Bioeng 42:1002-1013). In contrast, the T. fusca classic endoglucanase, Cel6A, only released twice as much soluble sugar as insoluble sugar. (Zhang et al. (2000) Eur J Biochem 267:244-252). Therefore, the processivity values for S. degradans Cel5H, Cel5G and Cel5J were most similar to that of the processive T. fusca Cel9A.

The identification of S. degradans Cel5H, Cel5G and Cel5J as endoglucanases is best supported by the effect of these enzymes on the viscosity of CMC solutions and the synergisms detected with a known exoglucanase. Exoglucanases have little effect on the overall degree of polymerization of CMC and its inherent viscosity whereas endoglucanases substantially reduce the degree of polymerization through random cleavage of the polymer with a corresponding decrease in viscosity. S. degradans Cel5H, Cel5G and Cel5J all rapidly reduced the viscosity of CMC solutions similarly to an endoglucanase. Endoglucanases act synergistically with exoglucanases by increasing the number of available ends. (Jeoh et al. (2006) Biotechnol Progr 22:270-277). As expected for endoglucanases, S. degradans Cel5H, Cel5G and Cel5J were synergistic with the known exoglucanase, T. fusca Cel6B. The absence of synergism with endoglucanases argues that the processive GH5 enzymes of S. degradans are not dependent upon other endoglucanases for activity and therefore lack cellobiohydrolase activity.

The processivity of the GH5 enzymes of S. degradans is unusual. In bacterial systems, processive endoglucanases have almost exclusively been found in the GH9 family. (Wilson, D. B. (2008) Ann NY Acad Sci 1125:289-297). Processive endoglucanase activity, however, has been suggested for another member of the GH5 family, Cel5A produced by the brown rot basidiomycete Gloeophyllum trabeum. (Cohen et al. (2005) Appl Environ Microbiol 71:2412-2417). As such, the processive endoglucanases of S. degradans should have distinctive structures. The catalytic sites of processive enzymes are typically associated with either tunnel conformations that enclose the substrate during catalysis or are located in deep clefts that partial enclose the substrate. (Breyer, W. A. and Matthews, B. W. (2001) Protein Sci 10:1699-1711). The catalytic sites of GH5 enzymes are cleft enzymes and the 7 residues that form the active site and cleft of the GH5 domains of Cel5G, Cel5H and Cel5J are conserved. (Ducros, V. et al (1995) Structure 3:939-949; Violot, S. et al (2005) J Mol Biol 348:1211-1224; Gilad, R. et al (2003) J Bacteriol 185:391-398). Phylogenetic analysis, however, revealed that other aspects of the primary sequence of these enzymes are sufficiently divergent to allow their segregation from the other families of GH5 cellulases found in S. degradans and other microorganisms. This infers that these enzymes have a distinct structure relative to other GH5 enzymes. As the GH5 cleft appears to be conserved in these enzymes, induced fit with the substrate could explain the processivity of these enzymes.

The processivity of most endoglucanases is dependent upon their associated CBM module. For example, the processivity of several bacterial GH9 cellulases is dependent upon the resident CBM3 (Gilad, R. et al (2003) J Bacteriol 185:391-398; Sakon et al. (1997) Nat Struct Biol 4:810-818). The processive GH5 cellulases of S. degradans are all linked to CBM6 modules via flexible linkers. (Howard, M. B. et al (2004) Protein Sci 13:1422-1425). These CBM6 modules exhibit properties typical of a Type B CBM that binds to individual polysaccharide chains and is consistent with the substrate bias of these enzymes towards amorphous cellulose as demonstrated by the high activity on CMC and PASC and the inability to breakdown cotton linters. The CBM6, however, was not necessary for activity or processivity. The truncated derivative of Cel5H, Cel5H′, retained greater than 78% of its activity on CMC, but did show a significant loss of activity on insoluble substrates. For example, the CBM6 was shown to contribute in the degradation of cotton linters, but while only acting on the amorphous regions of the substrate. The ratio of soluble to insoluble reducing sugars was not affected by the deletion of the CBM6.

Other systems lacking cellobiohydrolases have also been discovered where the role of processive endoglucanases is not well understood. (Wilson, D. B. (2008) Three microbial strategies for plant cell wall degradation. Ann NY Acad Sci 1125:289-297). For example, the cellulolytic system of Cytophaga hutchinsonii appears to be composed of 9 candidate endoglucanases containing either a GH5 or GH9 domain and 4 candidate β-glucosidases (Xie, G. et al (2007) Appl Environ Microbiol 73:3536-3546). Cellobiohydrolases are not obvious in this system. A similar system appears to be found in Fibrobacter succinogenes that contains five cellulases including Cel9D. (Qi et al. (2007) Appl Environ Microbiol 73:6098-6105). Cel9D is interesting in that it exhibits a wide range of synergistic interactions with other members of its own family. (Qi et al. (2008) J Bacteriol 190:1976-1984). C. hutchinsonii, F. succinogenes, and S. degradans all share the property of having a large number of predicted endoglucanases with relatively few cellobiohydrolases. Processive enzymes would enable these organisms to solubilize cellulose.

S. degradans degrades cellulose by at least two distinct mechanisms involving secreted GH5 cellulases. The primary mechanism appears to be through the activity of the processive endoglucanases that form cellobiose. The processive endoglucanases have the highest specific activity of the tested endoglucanases and are highly expressed. The high expression and activity of these enzymes explains the formation of cellobiose as the primary product of cellulose digestion by culture filtrates of S. degradans. Most likely this cellobiose is transported into the cell by a presently unknown transporter and convert to glucose by the activity of the cytoplasmic β-glucosidases Bgl1A or Bgl1B. A homolog to a cellobiose phosphorylase is also present in the genome opening the possibility of phosphorylytic cleavage to glucose 1-phosphate and glucose. At a lower rate, classic endoglucanases form cellodextrins that could be converted to glucose by the activity of the cellobiohydrolase Cel6A, the cellodextrinases Ced3A and Ced3B or the surface-associated β-glucosidase Bgl3C. This combination of mechanisms which utilizes processive endoglucanases, and a possible secondary mechanism utilizing phosphorylase activity to degrade cellulose, makes S. degradans a remarkable cellulolytic system.

This invention relates to a host cell (e.g., bacterium) modified to express one or more saccharifying enzymes such as processive endoglucanases. According to preferred embodiments, the host cell is modified to express the processive endoglucanases of Cel5A, Cel5G, Cel5H, Cel5J, and combinations and mixtures thereof. Cel5A, Cel5G, Cel5H, Cel5J are processive endoglucanases acting on the β-1-4 bonds. Cel5A is encoded by the nucleic acid sequence shown in the region of 3828980-3832483 of GenBank Accession No. NC_{—}007912. The GeneID is 3967764. The Cel5A protein is shown at GenBank Accession No. YP_{—}528472. Cel5G is encoded by the nucleic acid sequence shown in the region of 4130713-4132629 of GenBank Accession No. NC_{—}007912. The GeneID is 3965729. The Cel5G protein is shown at GenBank Accession No. YP_{—}528708. Cel5H is encoded by the nucleic acid sequence shown in the region of 4125970-4127862 of GenBank Accession No. NC_{—}007912. The GeneID is 3965710. The Cel5H protein is shown at GenBank Accession No. YP_{—}528706.1. Cel5J is encoded by the nucleic acid sequence shown in the region of 3151734-3153566 of GenBank Accession No. NC_{—}007912. The GeneID is 3968571. The Cel5J protein is shown at GenBank Accession No. YP_{—}527966.1.

In another preferred embodiment, a host cell is modified with a nucleic acid that expresses a homolog of Cel5A, Cel5G, Cel5H, or Cel5J. The isolated nucleic acid homolog of the invention comprises a nucleotide sequence which is at least about 40-60%, preferably at least about 60-70%, more preferably at least about 70-75%, 75-80%, 80-85%, 85-90%, or 90-95%, and even more preferably at least about 95%, 96%, 97%, 98%, 99%, or more identical to a nucleotide sequence that encodes any of Cel5A, Cel5G, Cel5H, or Cel5J.

The processive endoglucanases of the present invention act synergistically with exoglucanases by increasing the number of available ends. Accordingly, the host cell may be a cell that expresses or is known to express exoglucanases. According to some embodiments, the host cell may be modified to express one or more exoglucanases. In one preferred embodiment, the host cell is genetically modified to express both a processive endonuclease, for example, Cel5G, Cel5H and/or Cel5J as well as a beta-glucosidase, for example Bgl1A, Bgl1B, Bgl3C, Ced3A and/or Ced3B. Preferably, the host cell is a bacterial, plant, fungal or insect cell. In certain embodiments, the processive endonuclease or beta-glucosidase is a full length protein. In other embodiments, the processive endonuclease and/or beta-glucosidase lacks its N-terminus so that it cannot be secreted from the host cell. Preferably, the proteins lack their N-terminal signal peptide. In other embodiments, the processive endonuclease and/or beta-glucosidase only include the catalytic domain.

This invention relates to a host cell (e.g., bacterium) modified to express one or more saccharifying enzymes such as beta-glucosidases. According to preferred embodiments, the host cell is modified to express the beta-glucosidases of Bgl1A, Bgl1B, Bgl3C, Ced3A and/or Ced3B, and combinations and mixtures thereof. Bgl1A is encoded by the nucleic acid sequence shown in region 4563392-4564777 of GenBank Accession No. NC_{—}007912. The GeneID is 3966465. The Bgl1A protein is shown at GenBank Accession No. YP_{—}529070.1. Bgl1B is encoded by the nucleic acid sequence shown in the region complementary to 1805639-1806973 of GenBank Accession No. NC_{—}007912. The GeneID is 3968663. The Bgl1B protein is shown at GenBank Accession No. YP_{—}526868.1. Bgl3C is encoded by the nucleic acid sequence shown in th region of 3391161-3393761 of GenBank Accession No. ACCESSION NC_{—}007912. The GeneID is 3968493. The Bgl3C protein is shown at GenBank Accession No. YP_{—}528146.1. Ced3A is encoded by the nucleic acid sequence shown in the region complementary to 3155564-3158782 of GenBank Accession No. NC_{—}007912. The GeneID is 3968574. The Ced3A protein is shown at GenBank Accession No. YP_{—}527969.1. Ced3B is encoded by the nucleic acid sequence shown in the region complementary to 307565-310153 of GenBank Accession No. NC_{—}007912. The GeneID is 3968087. The Ced3B protein is shown at GenBank Accession No. YP_{—}525721.1.

In another preferred embodiment, a host cell is modified with a nucleic acid that expresses a homolog of Bgl1A, Bgl1B, Bgl3C, Ced3A and/or Ced3B. The isolated nucleic acid homolog of the invention comprises a nucleotide sequence which is at least about 40-60%, preferably at least about 60-70%, more preferably at least about 70-75%, 75-80%, 80-85%, 85-90%, or 90-95%, and even more preferably at least about 95%, 96%, 97%, 98%, 99%, or more identical to a nucleotide sequence shown above. The nucleic acid sequence may be modified so that the N-terminus is truncated. More specifically, the nucleic acid may be modified so that the N-terminal signal sequence is truncated. The nucleic acid sequence may also be modified so that only the catalytic domain of the protein is expressed.

According to some embodiments, the host cell is a marine γ-proteobacterium. According to preferred embodiments, the marine γ-proteobacterium is Saccharophagus degradans. The preferred strain of Saccharophagus degradans is the S. degradans strain 2-40 having the American Type Culture Collection accession number 43961. S. degradans is further described in: WO 2008/136997; WO 2008/033330; U.S. Patent Publication No. 2005/0136426; U.S. Patent Publication No. 2007/0292929; U.S. Pat. No. 7,384,772; and U.S. Pat. No. 7,365,180; the disclosures of which are incorporated herein by reference in their entireties. In certain preferred embodiments, S. degradans is transfected with an incQ plasmid (belonging to the incompatibility group Q). These plasmids include the plasmid pDSK600, among others. Preferably, the incQ plasmid provides resistance to kanamycin and/or spectinomycin. In certain embodiments, the promoter used with incQ plasmid is the 3xlacUV5 promoter. In other embodiments, the incQ plasmid, preferably the pDSK600 plasmid, is used with any inducible promoter that can regulate expression. In other embodiments, the expression vector for a

The present invention features a genetically modified host cell that overexpresses one or more processive endoglucanases (e.g., Cel5G, Cel5H, Cel5J, and combinations and mixtures thereof), the genetically modified host cell comprising one or more genetic modifications that provide for an increased level of processive endoglucanase activity.

The genetic modifications provide for production of processive endoglucanases at a level that is at least about 5% higher (e.g., from about 5% higher to 10³-fold, or more, higher) than the level of the processive endoglucanase in a control cell not comprising the genetic modification(s). According to some embodiments, the genetic modifications provide for production of processive endoglucanases at a level that is at least about 10% higher, at least about 50% higher, at least about 2-fold higher, at least about 3-fold higher, at least about 4-fold higher, at least about 5-fold higher, at least about 10-fold higher, at least about 20-fold higher, at least about 30-fold higher, at least about 40-fold higher, at least about 50-fold higher, at least about 60-fold higher, at least about 70-fold higher, at least about 80-fold higher, at least about 90-fold higher, at least about 100-fold higher, at least about 200-fold higher, at least about 300-fold higher, at least about 400-fold higher, or at least about 500-fold higher.

The genetic modifications provide for production of processive endoglucanases at a level that is at least about 5% higher (e.g., from about 5% higher to 10³-fold, or more, higher) than the level of the processive endoglucanase in a control cell not comprising the genetic modification(s). According to some embodiments, the genetic modifications provide for production of processive endoglucanases at a level that is at least about 10% higher, at least about 50% higher, at least about 2-fold higher, at least about 3-fold higher, at least about 4-fold higher, at least about 5-fold higher, at least about 10-fold higher, at least about 20-fold higher, at least about 30-fold higher, at least about 40-fold higher, at least about 50-fold higher, at least about 60-fold higher, at least about 70-fold higher, at least about 80-fold higher, at least about 90-fold higher, at least about 100-fold higher, at least about 200-fold higher, at least about 300-fold higher, at least about 400-fold higher, or at least about 500-fold higher.

DNA sequences encoding the saccharifying enzymes may be cloned into any suitable vectors for expression in intact host cells or in cell-free translation systems by methods well-known in the art. The particular choice of the vector, host, or translation system is not critical to the practice of the invention.

Several regulatory elements (e.g., promoters) have been isolated and shown to be effective in the transcription and translation of heterologous proteins in the various hosts. Such regulatory regions, methods of isolation, manner of manipulation, etc. are known in the art. Non-limiting examples of bacterial promoters include the β-lactamase (penicillinase) promoter; lactose promoter; tryptophan (trp) promoter; araBAD (arabinose) operon promoter; lambda-derived P₁ promoter and N gene ribosome binding site; and the hybrid tac promoter derived from sequences of the trp and lac UV5 promoters.

Expression and cloning vectors will likely contain a selectable marker, a gene encoding a protein necessary for survival or growth of a host cell transformed with the vector. The presence of this gene ensures growth of only those host cells that express the inserts. Typical selection genes encode proteins that 1) confer resistance to antibiotics or other toxic substances, e.g., ampicillin, neomycin, methotrexate, etc.; 2) complement auxotrophic deficiencies, or 3) supply critical nutrients not available from complex media, e.g., the gene encoding D-alanine racemase for Bacilli. Markers may be an inducible or non-inducible gene and will generally allow for positive selection. Non-limiting examples of markers include the ampicillin resistance marker (i.e., β-lactamase), tetracycline resistance marker, neomycin/kanamycin resistance marker (i.e., neomycin phosphotransferase), dihydrofolate reductase, glutamine synthetase, and the like. The choice of the proper selectable marker will depend on the host cell, and appropriate markers for different hosts as understood by those of skill in the art.

Vectors can contain one or more replication and inheritance systems for cloning or expression, one or more markers for selection in the host, e.g., antibiotic resistance, and one or more expression cassettes. The inserted coding sequences can be synthesized by standard methods, isolated from natural sources, or prepared as hybrids. Ligation of the coding sequences to transcriptional regulatory elements (e.g., promoters, enhancers, and/or insulators) and/or to other amino acid encoding sequences can be carried out using established methods.

Expression vectors for host cells ordinarily include an origin of replication (where extrachromosomal amplification is desired, as in cloning, the origin will be a bacterial origin), a promoter located upstream from the saccharifying enzyme coding sequences, together with a ribosome binding site (the ribosome binding or Shine-Dalgarno sequence is only needed for prokaryotic expression), RNA splice site (if the saccharifying enzyme DNA contains genomic DNA containing one or more introns), a polyadenylation site, and a transcriptional termination sequence. As noted, the skilled artisan will appreciate that certain of these sequences are not required for expression in certain hosts. An expression vector for use with microbes need only contain an origin of replication recognized by the intended host, a promoter which will function in the host and a phenotypic selection gene, for example a gene encoding proteins conferring antibiotic resistance or supplying an auxotrophic requirement.

Expression vectors, unlike cloning vectors, must contain a promoter which is recognized by the host organism. This is generally a promoter homologous to the intended host. Promoters most commonly used in recombinant DNA constructions include the β-lactamase (penicillinase) and lactose promoter systems (Chang et al., 1978, “Nature”, 275: 615; and Goeddel et al., 1979, “Nature” 281: 544), a tryptophan (trp) promoter system (Goeddel et al., 1980, “Nucleic Acids Res.” 8: 4057 and EPO Appl. Publ. No. 36,776) and the tac promoter (H. De Boer et al., 1983, “Proc. Nat'l. Acad. Sci. U.S.A.” 80: 21-25). While these are the most commonly used, other known microbial promoters are suitable. Details concerning their nucleotide sequences have been published, enabling a skilled worker operably to ligate them to DNA encoding a saccharifying enzyme in plasmid vectors. Promoters for use in prokaryotic expression systems also will contain a Shine-Dalgarno (S.D.) sequence operably linked to the DNA encoding the saccharifying enzymes of the present invention, i.e., the S.D. sequence is positioned so as to facilitate translation.

Host cells can be transformed, transfected, or infected as appropriate by any suitable method including electroporation, calcium chloride-, lithium chloride-, lithium acetate/polyethylene glycol-, calcium phosphate-, DEAE-dextran-, liposome-mediated DNA uptake, spheroplasting, injection, microinjection, microprojectile bombardment, phage infection, viral infection, or other established methods. Alternatively, vectors containing the nucleic acids of interest can be transcribed in vitro, and the resulting RNA introduced into the host cell by well-known methods, e.g., by injection. The cells into which have been introduced nucleic acids described above are meant to also include the progeny of such cells. Methods of transfection include nucleofection, electroporation, sonoporation, heat shock, magnetofection and proprietary transfection reagents such as Lipofectamine, Dojindo, GenePORTER, Hilymax, Fugene, jetPEI, Effectene or DreamFect.

Host cells carrying an expression vector (i.e., transformants or clones) are selected using markers depending on the mode of the vector construction. The marker may be on the same or a different DNA molecule, preferably the same DNA molecule. In prokaryotic hosts, the transformant may be selected, e.g., by resistance to ampicillin, tetracycline or other antibiotics. Production of a particular product based on temperature sensitivity may also serve as an appropriate marker.

It is further preferred that the isolated nucleic acid homolog of the invention encodes a saccharifying enzyme, or portion thereof, that is at least 80% identical to an amino acid sequence of any of Cel5A, Cel5G, Cel5H, or Cel5J, and that functions as a processive endoglucanase. In a more preferred embodiment, overexpression of the nucleic acid homolog in a host cell increases the host cell's yield of cellobiose.

For the purposes of the invention, the percent sequence identity between two nucleic acid or polypeptide sequences is determined using the Vector NTI 9.0 (PC) software package (Invitrogen, 1600 Faraday Ave., Carlsbad, Calif. 92008). A gap opening penalty of 15 and a gap extension penalty of 6.66 are used for determining the percent identity of two nucleic acids. A gap opening penalty of 10 and a gap extension penalty of 0.1 are used for determining the percent identity of two polypeptides. All other parameters are set at the default settings. For purposes of a multiple alignment (Clustal W algorithm), the gap opening penalty is 10, and the gap extension penalty is 0.05 with blosum62 matrix. It is to be understood that for the purposes of determining sequence identity when comparing a DNA sequence to an RNA sequence, a thymidine nucleotide is equivalent to a uracil nucleotide.

In another aspect, the invention relates to an isolated nucleic acid comprising a polynucleotide that hybridizes to the polynucleotide of any that encodes any of Cel5A, Cel5G, Cel5H, or Cel5J under stringent conditions. Preferably, an isolated nucleic acid homolog of the invention comprises a nucleotide sequence which hybridizes under highly stringent conditions to the nucleotide sequence that encodes any of Cel5A, Cel5G, Cel5H, or Cel5J and functions as a processive endoglucanase. In a further preferred embodiment, overexpression of the isolated nucleic acid homolog in a host cell increases a host cell's yield of cellobiose.

As used herein with regard to hybridization for DNA to a DNA blot, the term “stringent conditions” may refer to hybridization overnight at 60° C. in 10×Denhart's solution, 6×SSC, 0.5% SDS, and 100 μg/ml denatured salmon sperm DNA. Blots are washed sequentially at 62° C. for 30 minutes each time in 3×SSC/0.1% SDS, followed by 1×SSC/0.1% SDS, and finally 0.1×SSC/0.1% SDS. In a preferred embodiment, the phrase “stringent conditions” refers to hybridization in a 6×SSC solution at 65° C. As also used herein, “highly stringent conditions” refers to hybridization overnight at 65° C. in 10×Denharts solution, 6×SSC, 0.5% SDS, and 100 μg/ml denatured salmon sperm DNA. Blots are washed sequentially at 65° C. for 30 minutes each time in 3×SSC/0.1% SDS, followed by 1×SSC/0.1% SDS, and finally 0.1×SSC/0.1% SDS. Methods for nucleic acid hybridizations are described in Meinkoth and Wahl, 1984, Anal. Biochem. 138:267-284; Current Protocols in Molecular Biology, Chapter 2, Ausubel et al. Eds., Greene Publishing and Wiley-Interscience, New York, 1995; and Tijssen, 1993, Laboratory Techniques in Biochemistry and Molecular Biology Hybridization with Nucleic Acid Probes, Part I, Chapter 2, Elsevier, N.Y., 1993. Preferably, an isolated nucleic acid molecule of the invention that hybridizes under stringent or highly stringent conditions to a sequence of any that encodes any of Cel5A, Cel5G, Cel5H, or Cel5J and corresponds to a naturally occurring nucleic acid molecule. As used herein, a “naturally occurring” nucleic acid molecule refers to an RNA or DNA molecule having a nucleotide sequence that occurs in nature (e.g., encodes a natural polypeptide). In one embodiment, the nucleic acid encodes a naturally occurring processive endoglucanase.

Using the above-described methods, and others known to those of skill in the art, one of ordinary skill in the art can isolate homologs of the processive endoglucanases comprising amino acid sequences shown in any of Cel5A, Cel5G, Cel5H, or Cel5J. One subset of these homologs is allelic variants. As used herein, the term “allelic variant” refers to a nucleotide sequence containing polymorphisms that lead to changes in the amino acid sequences of a saccharifying enzyme and that exist within a natural population (e.g., a host cell species or variety). Such natural allelic variations can typically result in 1-5% variance in a saccharifying enzyme nucleic acid. Allelic variants can be identified by sequencing the nucleic acid sequence of interest in a number of different host cells, which can be readily carried out by using hybridization probes to identify the same HSRP genetic locus in those host cells. Any and all such nucleic acid variations and resulting amino acid polymorphisms or variations in a saccharifying enzyme that are the result of natural allelic variation and that do not alter the functional activity of a saccharifying enzyme, are intended to be within the scope of the invention.

Moreover, nucleic acid molecules encoding processive endoglucanases from the same or other species such as processive endoglucanase analogs, orthologs, and paralogs, are intended to be within the scope of the present invention. As used herein, the term “analogs” refers to two nucleic acids that have the same or similar function, but that have evolved separately in unrelated organisms. As used herein, the term “orthologs” refers to two nucleic acids from different species, but that have evolved from a common ancestral gene by speciation. Normally, orthologs encode polypeptides having the same or similar functions. As also used herein, the term “paralogs” refers to two nucleic acids that are related by duplication within a genome. Paralogs usually have different functions, but these functions may be related. Analogs, orthologs, and paralogs of a naturally occurring HSRP can differ from the naturally occurring HSRP by post-translational modifications, by amino acid sequence differences, or by both. In particular, orthologs of the invention will generally exhibit at least 80-85%, more preferably, 85-90% or 90-95%, and most preferably 95%, 96%, 97%, 98%, or even 99% identity, or 100% sequence identity, with all or part of a naturally occurring processive endoglucanase amino acid sequence, and will exhibit a function similar to a processive endoglucanase. Preferably, a saccharifying enzyme ortholog of the present invention functions as a processive endoglucanases.

In addition to naturally-occurring variants of a saccharifying enzyme sequence that may exist in the population, the skilled artisan will further appreciate that changes can be introduced by mutation into a nucleotide sequence of any that encodes any of Cel5A, Cel5G, Cel5H, or Cel5J, thereby leading to changes in the amino acid sequence of the encoded processive endoglucanase, without altering the functional activity of the processive endoglucanase. For example, nucleotide substitutions leading to amino acid substitutions at “non-essential” amino acid residues can be made in a sequence of any that encodes any of Cel5A, Cel5G, Cel5H, or Cel5J. A “non-essential” amino acid residue is a residue that can be altered from the wild-type sequence of one of the processive endoglucanases without altering the activity of said processive endoglucanase, whereas an “essential” amino acid residue is required for processive endoglucanase activity. Other amino acid residues, however, (e.g., those that are not conserved or only semi-conserved in the domain having processive endoglucanase activity) may not be essential for activity and thus are likely to be amenable to alteration without altering processive endoglucanase activity.

Accordingly, another aspect of the invention pertains to nucleic acid molecules encoding processive endoglucanases that contain changes in amino acid residues that are not essential for processive endoglucanase activity. Such processive endoglucanases differ in amino acid sequence from a sequence contained in any that encodes any of Cel5A, Cel5G, Cel5H, or Cel5J, yet retain at processive endoglucanase activity.

In one embodiment, the isolated nucleic acid molecule comprises a nucleotide sequence encoding a polypeptide, wherein the polypeptide comprises an amino acid sequence at least about 50-60% identical to the sequence of any of Cel5A, Cel5G, Cel5H, or Cel5J, more preferably at least about 60-70% identical to the sequence of any of Cel5A, Cel5G, Cel5H, or Cel5J, even more preferably at least about 70-75%, 75-80%, 80-85%, 85-90%, or 90-95% identical to the sequence of any of Cel5A, Cel5G, Cel5H, or Cel5J, and most preferably at least about 96%, 97%, 98%, or 99% identical to the sequence of any of Cel5A, Cel5G, Cel5H, or Cel5J.

An isolated nucleic acid molecule encoding a saccharifying enzyme having sequence identity with a polypeptide sequence of any of Cel5A, Cel5G, Cel5H, or Cel5J can be created by introducing one or more nucleotide substitutions, additions, or deletions into a nucleotide sequence that encodes any of Cel5A, Cel5G, Cel5H, or Cel5J, such that one or more amino acid substitutions, additions, or deletions are introduced into the encoded polypeptide. Mutations can be introduced into the sequence that encodes any of Cel5A, Cel5G, Cel5H, or Cel5J by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain.

Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine), and aromatic side chains (e.g., tyrosine, phenylalanine, tryptophan, histidine). Thus, a predicted nonessential amino acid residue in a saccharifying enzyme is preferably replaced with another amino acid residue from the same side chain family. Alternatively, in another embodiment, mutations can be introduced randomly along all or part of a saccharifying enzyme coding sequence, such as by saturation mutagenesis, and the resultant mutants can be screened for a saccharifying enzyme activity described herein to identify mutants that retain processive endoglucanase activity. Following mutagenesis of the sequence of any of Cel5A, Cel5G, Cel5H, or Cel5J, the encoded polypeptide can be expressed recombinantly and the activity of the polypeptide can be determined by analyzing processive endoglucanase as described herein elsewhere.

### Fermentation

The fermentation process may be carried out using any method known in the art. Fermentation may, therefore, be understood as comprising shake flask cultivation, small- or large-scale fermentation (including continuous, batch, fed-batch, or solid state fermentations) in laboratory or industrial fermenters performed in a suitable medium and under conditions allowing the fermentation of fermentable sugars into ethanol.

In the fermentation step, sugars, released from the plant cell wall polysaccharides are fermented to one or more organic substances, e.g., ethanol, by a fermentation organism, such as yeast, or fermenting organisms. According to preferred embodiments, the fermentation is carried out simultaneously with the enzymatic hydrolysis in the same vessels, again under controlled pH, temperature and mixing conditions. When saccharification and fermentation are performed simultaneously in the same vessel, the process is generally termed simultaneous saccharification and fermentation. The most widely used process in the art is the simultaneous saccharification and fermentation (SSF) process where there is no holding stage for the saccharification, meaning that the adding of the fermenting microorganism and lysis of the saccharifying microorganism occur in the same vessel.

During the fermentation stage, the combining of the impregnated pulp and fermenting organisms may be accomplished in a number of ways that one skilled in the art would readily be able to determine. A fermenting organism goes through different stages of growth including a lag phase, logarithmic phase, a stationary phase and a death phase. The length of the lag phase may vary depending on nutrition, growth conditions, temperature, and inoculation density. Also the lag phase may depend on whether or not the fermenting organism, such as yeast were acclimatized or directly added to a fermenter. Generally the lag phase is 6 to 9 hours. If a fermenting organism such as yeast can be kept in an active growth state, production of end products such as alcohol and particularly ethanol could be increased and fermentation time potentially decreased.

Therefore, in some embodiments the initial fermentation is conducted for a period of time that corresponds to the lag phase of the fermenting organism. In other embodiments, the initial fermentation step is conducted for a period of time between 2 to 40 hours, also between 2 to 30 hours, also between 2 to 25 hours, also between 5 and 20 and between 2 and 15 hours. In some embodiments, the initial fermentation time is greater than 2, 3, 4, 5, 6, 7, 8, 9, 10 or 15 hours but less than 36 hours.

In some embodiments, the initial fermentation is conducted at a temperature of at least about 5° C., 10° C., 15° C., 20° C., 25° C., 30° C., 35° C., 40° C., 45° C., 50° C., 55° C., 60° C., 65° C., 70° C., and 75° C. and also at a temperature of less than 70° C., less than 65° C. and less than 60° C. In other embodiments, the temperature will be between about 5-65° C., about 10-65° C., about 20-65° C., about 20-60° C., about 20-55° C., about 25-50° C., about 25-45° C., about 30-45° C., about 30-40° C. and about 35-45° C.

In some embodiments, the initial fermentation is conducted at a pH of between pH 3.0 and 7.0, between pH 3.0 and 6.5, between pH 3.0 and 6.0, between pH 3.0 and 5.0, between pH 3.5 and 5.5, between pH 3.5 and 5.0, between pH 3.5 and 4.5 or between pH 5.0 and 7.0. The exact temperature and pH used in accordance with any of the fermentation steps of the instant process depends upon the specific fermentable substrate and further may depend upon the particular plant variety, enzymes that are being used and the fermenting organism.

In some embodiments the total fermentation time of the fermentation process will be for about 24 to 336 hours, 24 to 168 hours, 24 to 144 hours, 24 to 108 hours; 24 to 96 hours, 36 to 96 hours, 36 to 72 hours, 48 to 72 hours, 72 to 120 hours, 120 to 168 hours and 168 to 336 hours. In a preferred aspect, the fermentation proceeds for 24-96 hours, such as typically 35-60 hours. In another preferred aspect, the temperature is generally between 26-40° C., in particular about 32° C., and the pH is generally from pH 3 to 6, preferably from about pH 4 to about 5. The fermenting organism are preferably applied in amounts of 10⁵ to 10¹², preferably from 10⁷ to 10¹⁰, especially 5×10⁷ viable cells count per ml of fermentation broth. During the ethanol producing phase the cell count (e.g., yeast cell count) should preferably be in the range from 10⁷ to 10¹⁰, especially around 2×10⁸. Specific examples of fermentation systems used with the invention are disclosed in U.S. Provisional Patent Application No. 61/156,158, filed on Feb. 27, 2009 and incorporated herein by reference in its entirety.

### Recovery

Following the fermentation, the organic substance of interest is recovered from the mash by any method known in the art. Such methods include, but are not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, distillation, or extraction. For example, in an ethanol fermentation, the alcohol is separated from the fermented plant cell wall polysaccharides and purified by conventional methods of distillation. Ethanol with a purity of up to about 96 vol. % ethanol can be obtained, which can be used as, e.g., fuel ethanol; drinking ethanol, i.e., potable neutral spirits; or industrial ethanol. According to preferred methods of ethanol production, following the fermentation the mash is distilled to extract the ethanol.

The yield of glucose (percent of the total solubilized solids) from a fermentable substrate may be at least about 60%, 65%, 70%, 75%, 80%, 85%, 90%, 91%, 92%, 93%, 94%, 95%, 96%, 97% and 98%. However, in a preferred embodiment, the glucose is continually produced and substantially all of the glucose is used in the process to produce an end-product, such as ethanol. In further embodiments, the final mash will include less than 1.0%, less than 0.8%, less than 0.5%, less than 0.2%, less than 0.15%, less than 0.1%, and less than 0.05% monosaccharides (w/v).

While the preferred end-product is an alcohol and particularly ethanol, other end-products may be obtained and these include without limitation, glycerol, ASA intermediates, 1,3-propanediol, butanol, isobutanol, acetic acid, lactic acid, oil, enzymes, antimicrobials, organic acids, amino acids and antibiotics.

In some embodiments, the yield of ethanol will be greater than 0.1%, 0.2%, 0.3%, 0.4%, 0.5%, 0.6%, 0.7%, 0.8%, 0.9%, 1%, 2%, 3%, 4%, 5%, 6%, 7%, 8%, 10%, 12%, 14%, 16%, 18% and 20% by volume. In other embodiments, at least 50%, 60%, 70%, 80% of the final ethanol yield is produced in the first 20, 22, 24, 26, 28 or 30 hours. In certain embodiments, the yield of ethanol will be greater than 3% and at least 5% of the final ethanol will be produced in the first 5 days. The ethanol obtained according to the fermentation process may be used as a fuel ethanol, potable ethanol or industrial ethanol.

The mash at the end of the fermentation may include from 0 to 30% residual cellulose or lignocellulose. In some embodiments, the mash may include at least 1%, 2%, 4%, 6%, 8%, 10%, 12% but less than 30%, less than 20% and less than 15% residual cellulose or lignocellulose.

The term “fermenting microorganism” refers to any microorganism suitable for use in a desired fermentation process. Suitable fermenting microorganisms according to the invention are able to ferment, i.e., convert, sugars, such as glucose, xylose, arabinose, mannose, galactose, or oligosaccharides, directly or indirectly into the desired fermentation product(s). Examples of fermenting microorganisms include fungal organisms, such as yeast. Preferred yeast includes strains of Saccharomyces spp., and in particular, Saccharomyces cerevisiae. Commercially available yeast include, e.g., Red Star®/Lesaffre Ethanol Red, FALI, SUPERSTART, GERT, and FERMIOL. Other microorganisms may also be used depending the fermentation product(s) desired. These other microorganisms include Gram positive bacteria, e.g., Lactobacillus such as Lactobacillus lactis, Propionibacterium such as Propionibacterium freudenreichii; Clostridium sp. such as Clostridium butyricum, Clostridium beijerinckii, Clostridium diolis, Clostridium acetobutylicum, and Clostridium thermocellum; Gram negative bacteria, e.g., Zymomonas such as Zymomonas mobilis; and filamentous fungi, e.g., Rhizopus oryzae. Bacteria that can efficiently ferment glucose to ethanol include, for example, Zymomonas mobilis.

According to preferred embodiments, the yeast is a Saccharomyces sp., Saccharomyces cerevisiae, Saccharomyces distaticus, Saccharomyces uvarum, Kluyveromyces, Kluyveromyces marxianus, Kluyveromyces fragilis, Candida, Candida pseudotropicalis, Candida brassicae, Clavispora, Clavispora lusitaniae, Clavispora opuntiae, Pachysolen, Pachysolen tannophilus, Bretannomyces, Bretannomyces clauseni.

It is well known in the art that the organisms described above can also be used to produce other organic substances, as described herein. Other examples might be clostridial strains for butanol or isobutanol production, algae for oil production, various bacteria for acetic acid production. According to some embodiments, the algae are used for the production of oils, which may then be used as a source to produce non-petroleum-based diesel fuel (e.g., biodiesel). Preferably, the alga is selected from spirogyra, cladophora, oedogonium, or a combination thereof. The production of biodiesel may be performed using any known method in the art. According to preferred embodiments, the saccharifying enzymes are inactivated using a heat and/or chemical process prior to the addition of algae.

A fermentation stimulator may also be used to improve the fermentation process, and in particular, the performance of the fermenting microorganism, such as, rate enhancement and ethanol yield. A “fermentation stimulator” refers to stimulators for growth of the fermenting microorganisms, in particular, yeast. Preferred fermentation stimulators for growth include vitamins and minerals. Examples of vitamins include multivitamins, biotin, pantothenate, nicotinic acid, meso-inositol, thiamine, pyridoxine, para-aminobenzoic acid, folic acid, riboflavin, and Vitamins A, B, C, D, and E. Examples of minerals include minerals and mineral salts that can supply nutrients comprising P, K, Mg, S, Ca, Fe, Zn, Mn, and Cu.

### DEFINITIONS

As used herein, the terms “transformed”, “stably transformed” or “transgenic” with reference to a cell means the cell has a non-native (heterologous) nucleic acid sequence integrated into its genome or as an episomal plasmid that is maintained through multiple generations.

As used herein, the term “expression” refers to the process by which a polypeptide is produced based on the nucleic acid sequence of a gene. The process includes both transcription and translation.

The term “introduced” in the context of inserting a nucleic acid sequence into a cell, means “transfection”, or “transformation” or “transduction” and includes reference to the incorporation of a nucleic acid sequence into a eukaryotic or prokaryotic cell where the nucleic acid sequence may be incorporated into the genome of the cell (for example, chromosome, plasmid, plastid, or mitochondrial DNA), converted into an autonomous replicon, or transiently expressed (for example, transfected mRNA).

By the term “host-cell” is meant a cell that contains a vector and supports the replication, and/or transcription or transcription and translation (expression) of the expression construct. Host cells for use in the present invention can be prokaryotic cells, such as E. coli, or eukaryotic cells such as yeast, plant, insect, amphibian, or mammalian cells. In general, host cells are S. degradans or other marine γ-proteobacterium.

The term “cellulase” refers to a category of enzymes capable of hydrolyzing cellulose polymers to shorter cello-oligosaccharide oligomers, cellobiose and/or glucose. Numerous examples of cellulases, such as exoglucanases, exocellobiohydrolases, endoglucanases, and glucosidases have been obtained from cellulolytic organisms, particularly including fungi, plants and bacteria.

The term “endoglucanase” is defined herein as an endo-1,4-(1,3; 1,4)-β-D-glucan 4-glucanohydrolase, which catalyses endohydrolysis of 1,4-β-D-glycosidic linkages in cellulose, cellulose derivatives (such as carboxymethyl cellulose and hydroxyethyl cellulose), lichenin, β-1,4 bonds in mixed β-1,3 glucans such as cereal β-D-glucans or xyloglucans, and other plant material containing cellulosic components. Endoglucanases digest the cellulose polymer at random locations, opening it to attack by cellobiohydrolases.

The exo-1,4-β-D-glucanases include both cellobiohydrolases and glucohydrolases.

The term “cellobiohydrolase” is defined herein as a 1,4-β-D-glucan cellobiohydrolase, which catalyzes the hydrolysis of 1,4-β-D-glucosidic linkages in cellulose, cellooligosaccharides, or any β-1,4-linked glucose containing polymer, releasing cellobiose from the reducing or non-reducing ends of the chain. Cellobiohydrolases sequentially release molecules of cellobiose from the ends of the cellulose polymer.

The term “glucohydrolase” is defined herein as a 1,4-β-D-glucan glucohydrolase, which catalyzes the hydrolysis of 1,4-linkages (O-glycosyl bonds) in 1,4-β-D-glucans so as to remove successive glucose units. Glucohydrolases liberate molecules of glucose from the ends of the cellulose polymer.

The term “β-glucosidase” is defined herein as a β-D-glucoside glucohydrolase, which catalyzes the hydrolysis of terminal non-reducing β-D-glucose residues with the release of β-D-glucose. Cellobiose is a water-soluble β-1,4-linked dimer of glucose. β-glucosidases hydrolyze cellobiose to glucose.

Analysis of zymograms and proteomic analyses of cultures may be used to reveal the identity of enzymes that are induced during growth on a particular substrate (e.g., glucose, Avicel™, oat spelt xylan, newsprint, whole and pulverized corn leaves, pulverized Panicum vigatum leaves, or any other known substrate). Induction of specific enzymes can be assessed by qRT-PCR. Nomenclature for specific enzymes is explained in further detail in U.S. application Ser. No. 11/121,154, filed on May 4, 2005 and published as U.S. Publication No. 2006/0105914, which is incorporated herein in its entirety.

The term “fermentation medium” will be understood to refer to a medium before the fermenting microorganism(s) is(are) added, such as, a medium resulting from a saccharification process, as well as a medium used in a simultaneous saccharification and fermentation process (SSF).

A “fermentable sugar” refers to mono- or disaccharides, which may be converted in a fermentation process by a microorganism in contact with the fermentable sugar to produce an end product. In some embodiments, the fermentable sugar is metabolized by the microorganism and in other embodiments the expression and/or secretion of enzymes by the microorganism achieves the desired conversion of the fermentable sugar.

As used herein, “monosaccharide” refers to a monomeric unit of a polymer such as starch wherein the degree of polymerization is 1 (e.g., glucose, mannose, fructose and galactose).

As used herein the term “starch” refers to any material comprised of the complex polysaccharide carbohydrates of plants, comprised of amylose and amylopectin with the formula (C₆H₁₀O₅)ₓ, wherein x can be any number.

The term “cellulose” refers to any cellulose-containing material. In particular, the term refers to the polymer of glucose (cellobiose) with the formula (C₆₁H₁₀O₅)ₓ, wherein x can be any number.

The term “slurry” refers to an aqueous mixture containing insoluble solids (may be used interchangeably with “pulp”).

The term “mash” refers to a mixture of a fermentable substrate in liquid used in the production of a fermented product and is used to refer to any stage of the fermentation from the initial mixing of the fermentable substrate or inoculated pulp and fermenting organisms through the completion of the fermentation run. Sometimes the terms “mash”, “fermentation broth”, and “fermentation medium” are used interchangeably. In some embodiments the term fermentation broth means a fermentation medium, which includes the fermenting organisms.

The terms “saccharifying enzyme” and “starch hydrolyzing enzymes” refer to any enzyme that is capable of converting starch to mono- or oligosaccharides.

The term “vessel” includes but is not limited to tanks, vats, bottles, flasks, bags, bioreactors and the like. In one embodiment, the term refers to any receptacle suitable for conducting the saccharification and/or fermentation processes encompassed by the invention.

“A”, “an” and “the” include plural references unless the context clearly dictates otherwise.

Numeric ranges are inclusive of the numbers defining the range.

The term “variant” refers to a protein or polypeptide in which one or more amino acid substitutions, deletions, and/or insertions are present as compared to the amino acid sequence of an protein or peptide and includes naturally occurring allelic variants or alternative splice variants of an protein or peptide. The term “variant” includes the replacement of one or more amino acids in a peptide sequence with a similar or homologous amino acid(s) or a dissimilar amino acid(s). There are many scales on which amino acids can be ranked as similar or homologous. (Gunnar von Heijne, Sequence Analysis in Molecular Biology, p. 123-39 (Academic Press, New York, N.Y. 1987.) Preferred variants include alanine substitutions at one or more of amino acid positions. Other preferred substitutions include conservative substitutions that have little or no effect on the overall net charge, polarity, or hydrophobicity of the protein. Conservative substitutions are set forth in the table below. According to some embodiments, the SPINT1 and TMPRSS4 polypeptides have at least 80%, 85%, 88%, 95%, 96%, 97%, 98% or 99% sequence identity with the amino acid sequences of the preferred embodiments.)

### Conservative Amino Acid Substitutions

| | |
|---|---|
| Basic: | arginine |
| | lysine |
| | histidine |
| Acidic: | glutamic acid |
| | aspartic acid |
| Uncharged Polar: | glutamine |
| | asparagine |
| | serine |
| | threonine |
| | tyrosine |
| Non-Polar: | phenylalanine |
| | tryptophan |
| | cysteine |
| | glycine |
| | alanine |
| | valine |
| | praline |
| | methionine |
| | leucine |
| | isoleucine |

The table below sets out another scheme of amino acid substitution:

| Original Residue | Substitutions |
|---|---|
| Ala | Gly; Ser |
| Arg | Lys |
| Asn | Gln; His |
| Asp | Glu |
| Cys | Ser |
| Gln | Asn |
| Glu | Asp |
| Gly | Ala; Pro |
| His | Asn; Gln |
| Ile | Leu; Val |
| Leu | Ile; Val |
| Lys | Arg; Gln; Glu |
| Met | Leu; Tyr; Ile |
| Phe | Met; Leu; Tyr |
| Ser | Thr |
| Thr | Ser |
| Trp | Tyr |
| Tyr | Trp; Phe |
| Val | Ile; Leu |

Other variants can consist of less conservative amino acid substitutions, such as selecting residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions that in general are expected to have a more significant effect on function are those in which (a) glycine and/or proline is substituted by another amino acid or is deleted or inserted; (b) a hydrophilic residue, e.g., seryl or threonyl, is substituted for (or by) a hydrophobic residue, e.g., leucyl, isoleucyl, phenylalanyl, valyl, or alanyl; (c) a cysteine residue is substituted for (or by) any other residue; (d) a residue having an electropositive side chain, e.g., lysyl, arginyl, or histidyl, is substituted for (or by) a residue having an electronegative charge, e.g., glutamyl or aspartyl; or (e) a residue having a bulky side chain, e.g., phenylalanine, is substituted for (or by) one not having such a side chain, e.g., glycine. Other variants include those designed to either generate a novel glycosylation and/or phosphorylation site(s), or those designed to delete an existing glycosylation and/or phosphorylation site(s). Variants include at least one amino acid substitution at a glycosylation site, a proteolytic cleavage site and/or a cysteine residue. Variants also include proteins and peptides with additional amino acid residues before or after the protein or peptide amino acid sequence on linker peptides. The term “variant” also encompasses polypeptides that have the amino acid sequence of the proteins/peptides of the present invention with at least one and up to 25 (e.g., 5, 10, 15, 20) or more (e.g., 30, 40, 50, 100) additional amino acids flanking either the 3′ or 5′ end of the amino acid sequence.

The headings provided herein are not limitations of the various aspects or embodiments of the invention, which can be had by reference to the specification as a whole.

The following examples are illustrative, but not limiting, of the methods and compositions of the present invention. Other suitable modifications and adaptations of the variety of conditions and parameters normally encountered in therapy and that are obvious to those skilled in the art are within the spirit and scope of the embodiments.

### EXAMPLES

The following examples further support, but do not exclusively represent, preferred embodiments of the present invention.

### Example 1

### Genotyping Methods

The growth rate of S. degradans was measured when it was cultured on different substrates. The basic media was composed of (2.3% Instant Ocean, 0.05% Yeast Extract, 0.05% NH₄Cl, 15 mM Tris, pH 6.8). The final concentration of each carbon source were 0.2% for Glucose, Xylose, Cellobiose, Arabinose, Xylan, Avicel™ and 1.0% for Newsprint, Switchgrass, and corn leaves. S. degradans grew on all plant material it was grown on.

A Zymogram was performed to find which glucanases were induced during growth on various cell wall polymers (FIG. 2). Cells were grown to an OD₆₀₀ of 0.3-0.5 in media containing glucose as the sole carbon source, harvested and transferred to the same volume of media containing the indicated inducer. Samples were removed at the indicated times and proteins in samples normalized to OD₆₀₀ were fractionated by standard SDS-PAGE in which either 0.1% barley β-glucan or HE cellulose was included in the resolving gel. Gels were incubated in refolding buffer (20 mM PIPES [piperazine-N,N′-bis(2-ethanesulfonic acid)] buffer [pH 6.8], 2.5% Triton X-100, 2 mM dithiothreitol, 2.5 mM CaCl₂) for 1 h at room temperature and then held overnight in fresh refolding buffer at 4° C. The gels were transferred to PIPES buffer, incubated at 37° C., and stained in 0.25% Congo red. Calculated masses are shown on the left in kDa. Different glucanases were expressed in the presence of different plant materials or carbohydrate sources used.

The expression of cellulolytic enzymes during growth on glucose and cell wall polymers was determined (FIG. 3). S. degradans was cultured on glucose to OD₆₀₀ 0.3-0.4, harvested and transferred to the same volume medium containing the indicated substrate. After 10 hours the RNA was isolated using the RNA PROTECT™ Bacteria Reagent (Qiagen) and RNEASY™ Mini kit (Qiagen). The cDNA was synthesized using the QIANTITECT™ Reverse Transcription Kit. The 120-200 bp fragments of each indicated gene or two control genes for Guanylate kinase and Dihydrofolate reductase were amplified using the SYBR Green™ master mix kit (Roche) and a LIGHT CYCLE® 480 (Roche). The bars shown with numbers above them are presented at 1/10 scale. Different celluloytic enzymes were induced by different plant materials or carbohydrate sources.

### Example 2

### Measurement of Increase in Expression of Xyn10A, Xyn10B, Xyn11A and Xyn11B in Response to Growth of S. Degradans on Xylan

Primers were designed for six target genes: xyn10A-D and xyn11A-B along with two house keeping genes: dihydrofolate reductase and guanylate kinase. S. degradans was cultured in glucose media until OD₆₀₀ reached 0.370-0.400. The 0 hour time point was taken and the cultures were transferred to xylan media for 10 hour time course experiments. A second culture was transferred back to glucose as a control. Samples were taken at 0, 2, 4 and 10 hours from both the xylan and glucose cultures.

RNA from each sample was purified using RNAprotect™ bacteria reagent (Qiagen) and Rneasy MiniKit. The isolated mRNA was transformed using QuantiTech™ reverse transcriptase and expression patterns were analyzed using LightCycler Pro™ pRT-PCR.

As shown in FIG. 6, xyn10A, xyn10B, xyn10D, xyn11A and xyn11B all had greater mRNA expression at 2, 4, and 10 hours after exposure to xylan. The increases were the greatest for xyn10A, xyn10B, xyn11A and xyn11B. As shown in FIG. 7, the highest fold induction of mRNA expression at 2 hours of culture of S. degradans on xylan, was for xyn11A and xyn11B. Xyn10A had the highest induction at 4 hours. At 10 hours, xyn10A, xyn10B, xyn11A and xyn11B all had higher fold induction.

As S. degradans increases expression of these proteins when it is exposed to xylan, and with the sequence homology these proteins have to known hemicellulase genes, Xyn10A, Xyn10B, Xyn10D, Xyn11A and Xyn11B are functional hemicellulases that can be used to break down hemicellulose.

### Example 3

### Secreted Enzymes of S. Degradans Produce Cellobiose

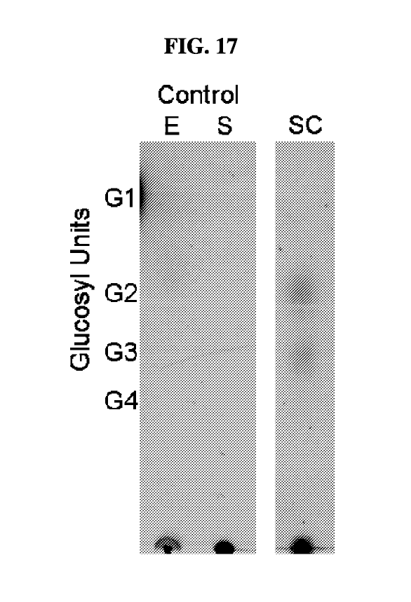

The cellulolytic system of S. degradans is capable of utilizing pure cellulose for its growth and metabolism. To determine the initial products of cellulose degradation, the secreted enzymes of S. degradans were allowed to degrade cellulose and the products of the reaction monitored by thin-layer chromatography (FIG. 17). Irrespective of the cellulosic substrate or reaction time, cellobiose was the primary product. This opened the possibility that one or more of the GH5 enzymes could generate cellobiose and/or cellotriose.

### Example 4

### Activity of GH5 Glucanases of S. Degradans

To understand the mechanism for degradation of cellulose, the biochemical activity for each of the GH5 cellulases predicted for the S. degradans cellulolytic system was evaluated. Three of the originally annotated endoglucanases, Cel5H, Cel5G and Cel5J were shown to be processive. This is a new activity for this family of enzymes and suggests that this bacterium utilizes a novel mechanism to degrade cellulose. In this mechanism, these highly expressed and active processive GH5 endoglucanases degrade cellulose to cellobiose. The cellobiose is then converted to glucose by the activity of β-glucosidases or cellobiose phosphorylase.

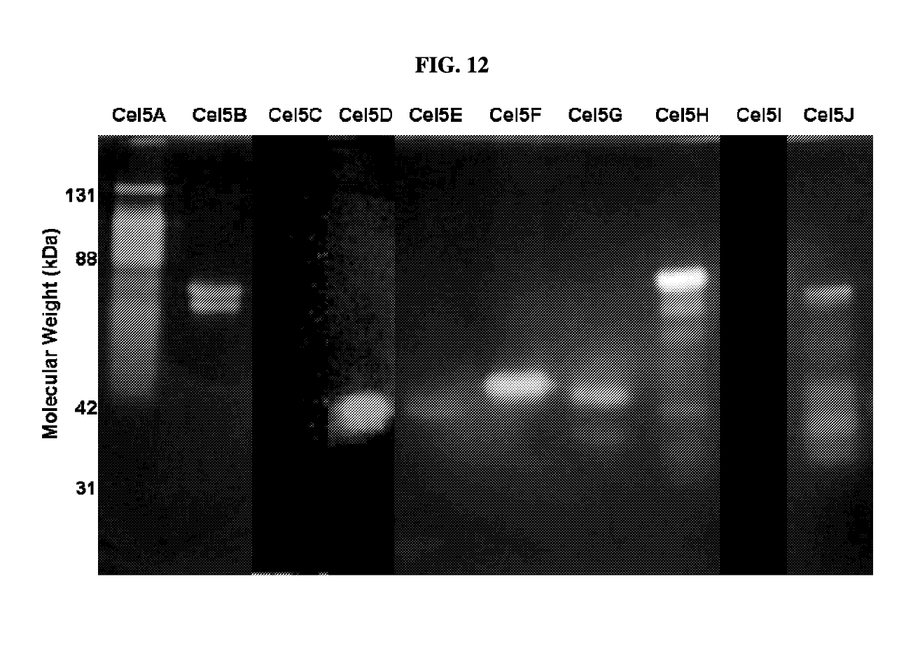

In order to test the proposed biochemical activities of the annotated GH5 glucanases, genes for each of the annotated GH5 glucanases were amplified from the S. degradans 2-40 genome by PCR and cloned into the T7 expression system carried by pET28b (Table S2). After transformation into E. coli Rosetta2™ (DE3), expression of the cloned genes was induced by IPTG (Isopropyl β-D-1-thiogalactopyranoside) and enzyme activity in cell lysates assessed using β-glucan and HE (hydroxyethyl) cellulose zymograms. Most of the expressed GH5 glucanases exhibited endoglucanase activity in β-glucan and HE cellulose zymograms consistent with their annotation as cellulases (FIG. 12). Endoglucanase activity was detected in each case as zones of clearing in Congo Red-stained gels. Cel5B, Cel5C, Cel5E, Cel5F, and Cel5H were comparatively stable. In contrast, Cel5A, Cel5D, Cel5G, Cel5I, and Cel5J were unstable. Residual full length polypeptides as well as the fragments of Cel5A, Cel5G and Cel5J sufficient to carry the catalytic domain retained cellulase activity. None of the fragments derived from Cel5C or Cel5I exhibited evidence of cellulase activity on any substrate. Use of protease inhibitors, alternative protease-deficient host strains, a variety of cell lysis protocols and alternative cloning strategies did not improve the stability of any of these modular enzymes.

TABLE S2
Primers used in this study

| | Restric- | |
|---|---|---|
| | tion | |
| Primer | site | Nucleotide sequence |
| Cel5A-F | BamHI | CCCGGATCCCATGCAAAGCACTGCAGCGGTA |
| Cel5A-R | EcoRI | CCGAATTCCACGGTGCTTGTGCTGCGTATTC |
| Cel5B-F | BamHI | CCCGGATCCTGGTGGCGATGCTTTAGCGTGC |
| Cel5B-R | EcoRI | CCGAATTCATCGGGTTTGGCGCCACTAATAC |
| Cel5C-F | BamH I | CCCGGATCCCGAAGCGCTTTACCCAAGCTAC |
| Cel5C-R | EcoR I | CCGAATTCCCTTCCATAATGGCATCTAG |
| Cel5D-F | BamH I | CCCGGATCCATCAAGCTCTAGTTCGTCGTCT |
| Cel5D-R | EcoR I | CCGAATTCCCGGCATTGATAATTGCGTT |
| Cel5E-F | BamHI | GCGAATTCAACGCGGTATTTCTAGGTCC |
| Cel5E-R | HindIII | CGCAAGCTTCGTCCAAATAGGTACTTGGTTCTAGC |
| Cel5F-F | BamHI | CCCGGATCCTGCAAATAACAGCGCCCCATCA |
| Cel5F-R | EcoRI | CCGAATTCCCAGCTTCTAGCATAACCTGTTT |
| Cel5G-F | BamHI | CCCGGATCCCGTAGCGCCGTTAACCGTAGAT |
| Cel5G-R | EcoRI | CCGAATTCGATGAGCTTGAGGAGGAACT |
| Cel5H-F | BamHI | CCCGGATCCAATTCTTAGCGGTGGCCAGCAA |
| Cel5H-R | XhoI | CCCGCTCGAGCCAGCTACCAAATTGCAGGGTGT |
| Cel5I-F | BamH I | CCGGATCCTGGTGGTGGAGTATTCCGCGTA |
| Cel5I-R | Eco R I | CCGAATTCCGAGAATCGAAGTCTAACCAAC |
| Cel5J-F | BamHI | CCCGGATCCCGTGCCAGCAATGTCCGTACAA |
| Cel5J-R | EcoRI | CCGAATTCCCGAAGCCACCACTAGTAATACC |

Significant differences were noted, however, in the apparent activity of these cellulases. Although each polypeptide appeared to be expressed at similar levels from the pET28b expression system in Rosetta2™ (DE3) transformants, some lysates had to be diluted significantly in order to see discrete regions of activity in zymograms. The highest activity seemed to be associated with Cel5G, Cel5H and Cel5J. Cell lystates of Rosetta2™ (DE3) transformants expressing these enzymes had to be diluted at least 10⁵ before a well-defined zone of activity typical of an individual polypeptide could be identified (FIG. 12).

### Example 5

### Purification and Properties of Cel5H

Because the apparent specific activity of Cel5H seemed to be at least 100-fold higher than the other cellulases and the polypeptide appeared to be stable in lysates, it was chosen for further analysis. To determine the biochemical properties of Cel5H, the polypeptide was purified to near homogeneity from Rosetta2™ (DE3) transformants by nickel-NTA (nitriltriacetic acid) chromatography employing the 6×-His tags created when the gene was cloned into pET28b. In order to establish the appropriate assay conditions, optimal temperature, pH and ionic conditions were determined. Cel5H exhibited a pH optimum of 6.5 and retained greater than 84% of its activity in the range between pH 6.0-7.0. Activity increased linearly up to 50° C. but lost activity at higher temperatures. Addition of salt enhanced the activity of the enzyme. A 2.5 fold increase in activity was observed when 1% Instant Ocean™ was included in the assay mixture. This effect was complex as 1% NaCl only partially substituted for Instant Ocean (1.9-fold stimulation) and the metal salts included in Instant Ocean were not stimulatory individually or in combination with 1.0% NaCl. The specific activity of purified Cel5H was established on soluble carboxymethyl cellulose (CMC) as well as substrates of increasing crystallinity (Table S1). The activity was highest on CMC but significant activity was retained on filter paper and Avicel™.

TABLE S1
Specific Activity of S. degradans Cel5H on Cellulosic Substrates

| | Specific activity on the indicated substrate | | | |
|---|---|---|---|---|
| | (μmol reducing sugar × minute⁻¹ × μmol cellulase^{-1a,b}) | | | |
| Cellulase | | Swollen | | |
| (kDa)^{c} | CMC^{d} | Cellulose^{d} | Filter Paper^{d} | Avicel ™^{d} |
| Cel5H (72) | 643 ± 23% | 792 ± 15% | 2.21 ± 5% | 1.45 ± 10% |
| Cel5H′ (35) | 500 ± 2% | 545 ± 15% | 0.92 ± 11% | 0.66 ± 11% |

^{a}Reducing sugar released from CMC and swollen cellulose was measured using a DNS assay relative to a cellobiose standard curve whereas glucose released from filter paper and Avicel ™ was measured using the glucose oxidase method.
^{b}milligrams of protein measured using BSA as the reference
^{c}The size of the largest polypeptide in a barley-β-glucan zymogram (kDa)
^{d}The approximate degree of crystallinity for each substrate (% crystallinity, 14): carboxymethyl cellulose CMC (soluble), PASC (10-20%), filter paper (50%), and Avicel ™ *(70%).

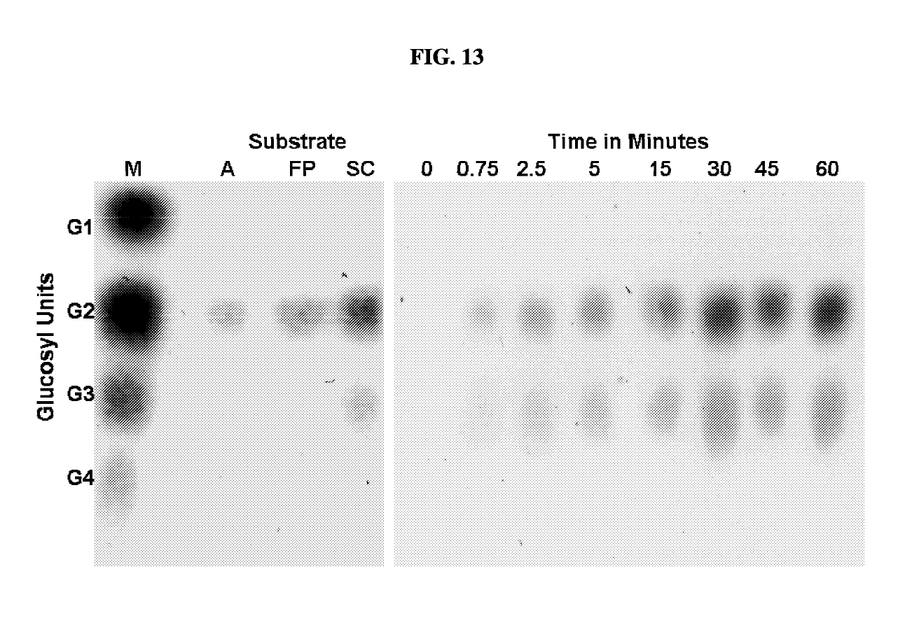

Product Analysis: The products released by Cel5H were determined using thin layer chromatography (TLC). Cellobiose was the primary product formed during digestion of swollen cellulose, filter paper, and Avicel™ (FIG. 13). Because the apparent accumulation of cellobiose could be due to the length of the digestion, shorter digestions were performed. Even at 45 seconds cellobiose was the primary product detected. In no case could products larger than cellotriose be detected.

Cel5H is Processive: The accumulation of cellobiose as the primary reaction product could be an indication of processivity. To evaluate the processivity of Cel5H, the ratio of soluble and insoluble products was measured and compared to that of T. fusca Cel9A (processive endoglucanase) and T. fusca Cel6A (classical endoglucanase) (gifts of D. Wilson, 7). Like the processive T. fusca Cel9A and in contrast to the classical endoglucanase T. fusca Cel6A (11), 82% of the products formed by the activity of the S. degradans Cel5H were soluble (Table S4). For both T. fusca Cel9A and S. degradans Cel5H the ratio of soluble products to insoluble products was excess of 4.

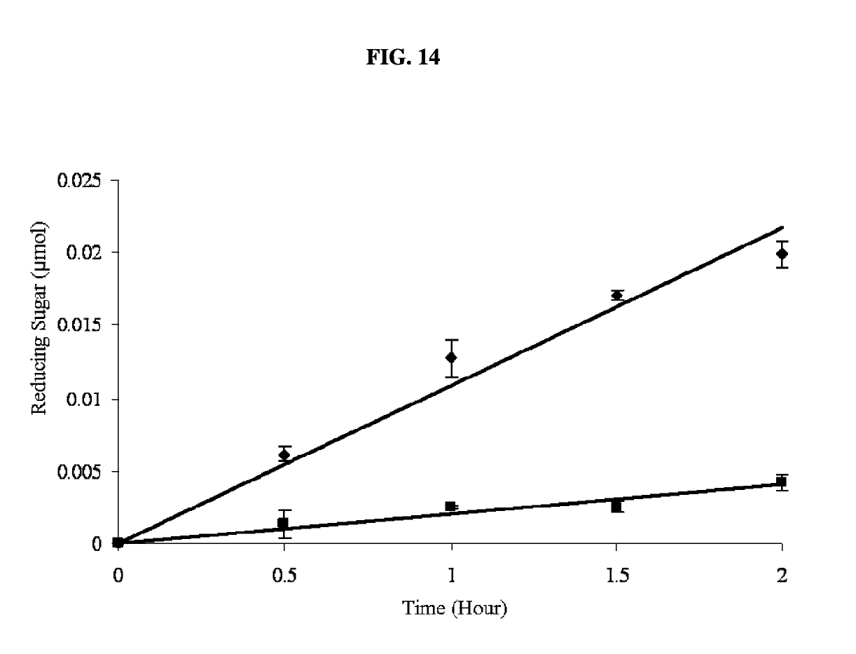

An additional experiment was performed to verify the processivity of Cel5H. Both soluble and insoluble ends were measured during a 2 hr time course (FIG. 14). The rates of soluble and insoluble product formation were divergent. Soluble sugar was released at a rate greater than 4 times faster than insoluble ends consistent with the data of Table S4.

TABLE S4
Processivity of S. degradans Cel5H

| | | | Soluble | Insoluble |
|---|---|---|---|---|
| | Specific | Processive | Reducing | Reducing |
| Cellulase^{a} | Activity^{b} | Ratio^{c} | Sugar^{d} | Sugar^{d} |
| T. fusca Cel9A | 0.70 ± 0.014 | 4.72 ± 0.43 | 82.5% | 17.5% |
| (Proc. Endo) | | | | |
| T. fusca Cel6A | 1.33 ± 0.14 | 2.55 ± 0.28 | 71.9% | 28.1% |
| (Endo) | | | | |
| S. degradans | 1.66 ± 0.07 | 4.26 ± 0.71 | 81.4% | 18.6% |
| Cel5H | | | | |
| S. degradans | 0.92 ± 0.01 | 4.42 ± 1.07 | 81.1% | 18.9% |
| Cel5H′ | | | | |

^{a}0.1 nmol of each cellulase was used in each 2 h digestion of filter paper.
^{b}The specific activity is reported as μmol reducing sugar × minute⁻¹ × μmol cellulase⁻¹.
^{c}The processive ratio is defined as the μmol soluble reducing sugar (cellobiose standard) divided by μmol insoluble sugar (glucose standard).
^{d}The mass fractions of soluble and insoluble products given as percentages.

Cel5H acts from the nonreducing end of cellulose: The processivity of Cel5H opens the possibility that there is directionality to its activity similar to cellobiohydrolases specific to either the nonreducing or reducing end of the cellulose polymer. To determine whether Cel5H acts from the non-reducing or reducing end, degradation of para-nitrophenol-cellobioside (pNP-cellobioside) was monitored. The activity of Cel5H released pNP and cellobiose from the substrate. As the pNP group was located at the reducing end of the cellobiose unit in the substrate, these results indicate that Cel5H acts from the non-reducing end of the polymer to release cellobiose.

### Example 6

### Role of CBM6 in the Activity and Processivity of Cel5H

To determine the contribution of the resident CBM6 of Cel5H to its activity and processivity, the activity of the full length polypeptide was compared to that of its truncated derivative consisting of the GH5 catalytic domain constructed by specific amplification of the catalytic domain (Cel5H′). The specific activity of Cel5H′ was 69% that of Cel5H on amorphous PASC and 78% on soluble CMC. On the crystalline substrates, 42% of the activity was retained on filter paper and 46% on Avicel™ (Table S1). The ratio of soluble to insoluble products, however, remained constant and was indistinguishable from that of Cel5H. Thus, processivity of the enzyme resides with the catalytic domain.

### Example 7

### Cel5H is an Endoglucanase

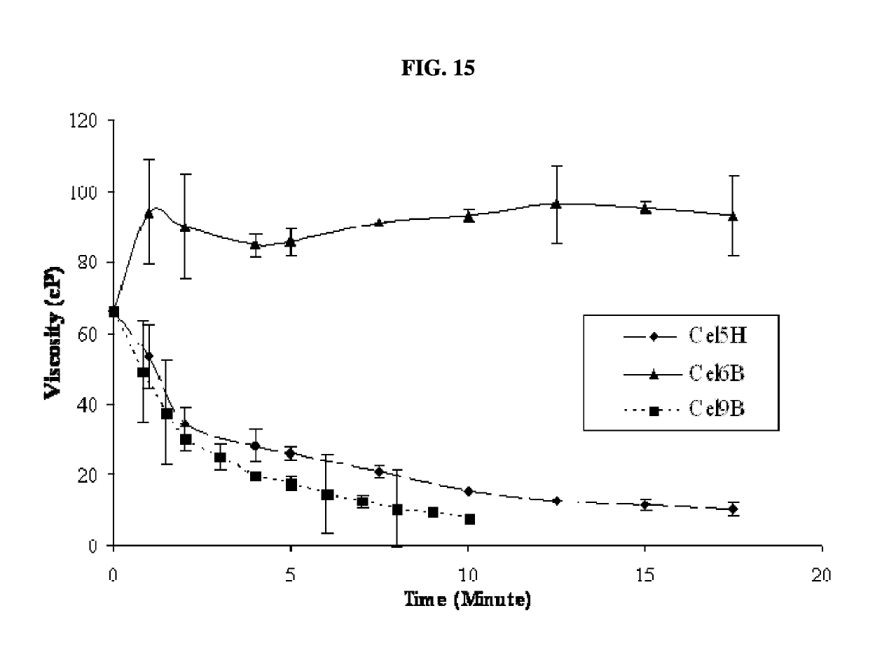

The observation of processivity was surprising for this GH5 family enzyme as the activity of the enzymes carrying this domain have previously been classified as endoglucanases (Lo Leggio, L. and Larsen, S. (2002) The 1.62 angstrom structure of Thermoascus aurantiacus endoglucanase: completing the structural picture of subfamilies in glycoside hydrolase family 5. FEBS Lett 523:103-108). To evaluate whether Cel5H is an endoglucanase, its effect on the viscosity of CMC solutions was monitored. When a soluble polymer such as CMC is cleaved randomly, the degree of polymerization dramatically decreases and viscosity is reduced whereas exo-acting enzymes have little effect on viscosity. (Wilson, D. B. (2004) Chem Rec 4:72-82). The exo-acting cellulase, T. fusca Cel6B, had no significant effect on the viscosity of CMC as expected (FIG. 15). In contrast, S. degradans Cel5H rapidly decreased the viscosity of CMC similar to the endoglucanase T. fusca Cel9B, thus supporting the prediction that Cel5H is an endoglucanase.

Synergistic interactions between cellulases also provide an indication of the activity of the enzymes. Endo-acting cellulases act synergistically with exo-acting enzymes by increasing the availability of reducing ends on the substrate for the exo-acting enzyme. (Jeoh et al. (2006) Effect of cellulase mole fraction and cellulose recalcitrance on synergism in cellulose hydrolysis and binding. Biotechnol Progr 22:270-277). S. degradans Cel5H acted synergistically with the exoglucanase T. fusca Cel6B, producing at least 30% more product when acting together than each individually (Table S3). In contrast anti-synergism was observed with the endoglucanase Cel9B with combined activities 15-25% lower than theoretical as seen previously in the interaction of classical endoglucanases. (Jeoh et al. (2006) Effect of cellulase mole fraction and cellulose recalcitrance on synergism in cellulose hydrolysis and binding. Biotechnol Progr 22:270-277). These are consistent with the identification of Cel5H as a processive endoglucanase.

TABLE S3
Synergy of S. degradans and T. fusca cellulases

| | | Yield of Glucose (μg) | | | |
|---|---|---|---|---|---|
| | Nanomoles | Independent | Theoretical | Actual | |
| Cellulase^{a} | Cellulase | Yield^{b} | Yield^{c} | Yield | DoS^{d} |
| S. degradans Cel5H | 1 | 12.6 ± 0.90 | | | |
| T. fusca Cel9B | 0.1 | 5.91 ± 0.49 | 18.5 | 14.3 ± 0.44 | 0.77 |
| T. fusca Cel6B | 0.1 | 2.65 ± 0.02 | 15.3 | 20.1 ± 0.89 | 1.31 |
| S. degradans Cel5G | 1 | 10.5 ± 1.32 | | | |
| T. fusca Cel9B | 0.1 | 5.91 ± 0.49 | 16.4 | 14.1^{e} | 0.86 |
| T. fusca Cel6B | 0.1 | 2.65 ± 0.02 | 13.2 | 20.4 ± 0.95 | 1.55 |

^{a}T. fusca Cel9B (Endoglucanase) and T. fusca Cel6B (Exoglucanase)
^{b}The independent yield was measured after two hours as described for the filter paper assay
^{c}The sum of the μg glucose produced for both the S. degradans and the T. fusca cellulases
^{d}DoS: The degree of synergy: the actual yield divided by the theoretical yield
^{e}Representative of single trial due to lack of T. fusca cellulase

### Example 8

### Cel5H Acts on Amorphous Cellulose

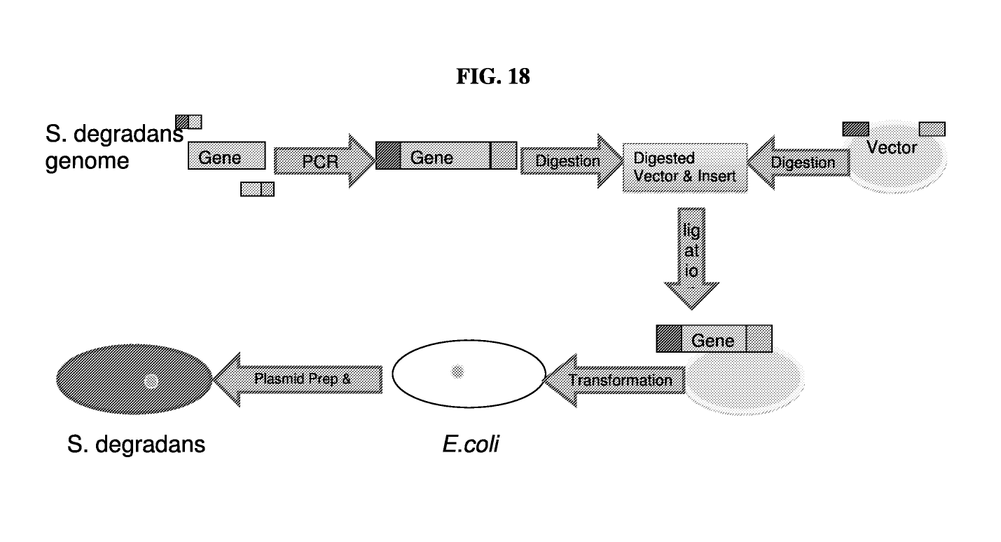

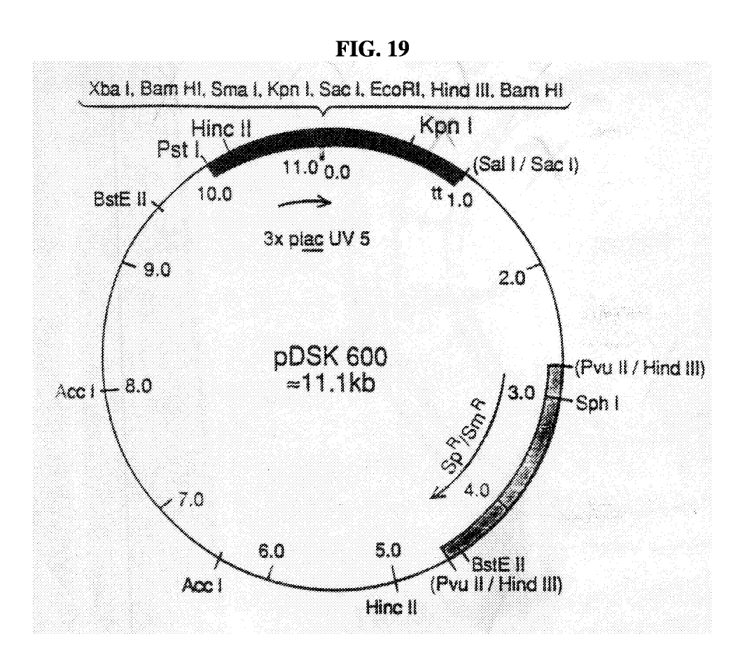

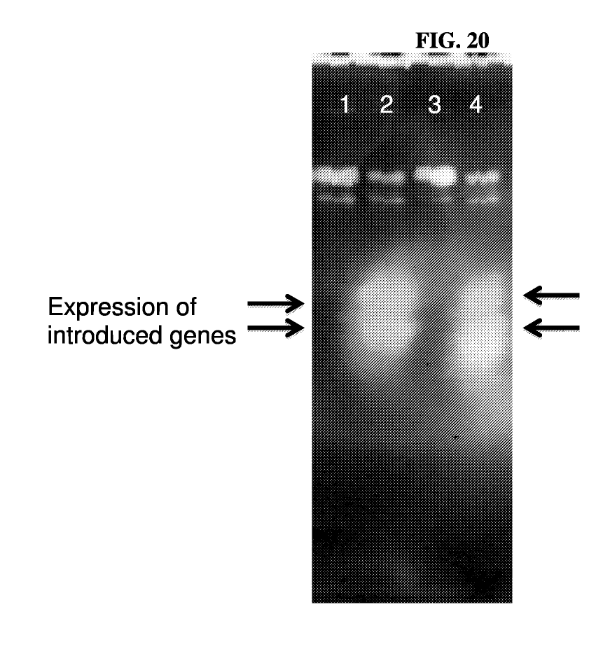

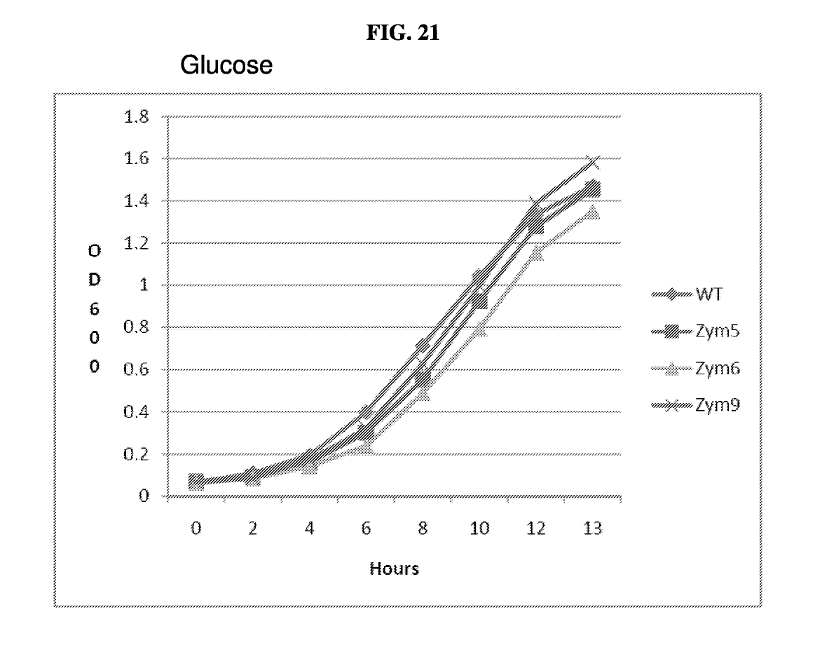

The comparative high activity of Cel5H on crystalline substrates, such as Avicel™, suggested that this enzyme may be acting on crystalline cellulose like many well known cellobiohydrolases. To investigate this hypothesis, an extended degradation was performed using highly crystalline hydrolyzed cotton linters (>90% crystalline) as the substrate. (Zhang et al. (2006) Outlook for cellulase improvement: Screening and selection strategies. Biotechnol Adv 24:452-481). After 150 hours only 0.1% percent of the hydrolyzed cotton linters were converted to cellobiose (FIG. 18). The preparation, however, retained similar levels of activity on CMC through the course of the experiment. Similar levels of digestion were obtained with Cel5H. In contrast, a commercial preparation derived from H. jecorina known to degrade crystalline cellulose (Accelerase 1000; Genencor) degraded greater than 7% of the substrate in 24 hours. These observations are most consistent with a substrate bias of Cel5H towards the amorphous regions of cellulose consistent with the presence of a CBM6.

### Example 9

### Phylogenetic Analysis of the S. Degradans GH5 Domains

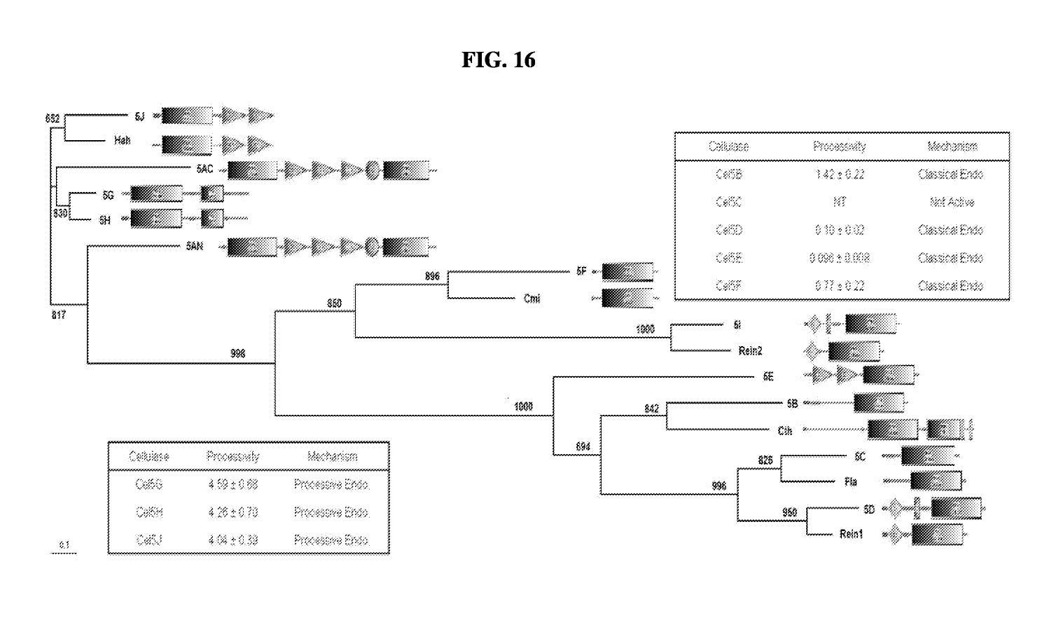

To understand the relationships between Cel5H and the other GH5 endoglucanases of S. degradans, phylogenetic relationships were determined by nearest neighbor joining. Significant differences were apparent in the organization of the structural features of S. degradans GH5 endoglucanases that precluded the use of full length polypeptides in the phylogenetic analyses. Instead, the GH5 domain of each cellulase together with that of its closest homolog in the NR database was used in the analyses. Two distinct clades of GH5 endoglucanases were apparent (FIG. 16). The majority of the endoglucanases carry a GH5 domain near the carboxy terminus of the host polypeptide and segregate into a single clade with strong bootstrap support. The first clade included Cel5A_{N}, Cel5B, Cel5C, Cel5D, Cel5E, CelF, and Cel5I as well as their homologs. The second clade comprised Cel5A_{C}, Cel5G, Cel5H, and Cel5J. Most of the enzymes associated with the second clade carry a GH5 near the amino terminus of the polypeptide and a CBM6 near the carboxy terminus. Of particular interest were Cel5G and Cel5H. Both enzymes exhibited strong similarities in sequence and domain organization, including the 7 conserved catalytic residues of the GH5 catalytic domain, differing only in the length and sequence of the linker region. No full length homologs to Cel5G/Cel5H were detected in the databases. The distinct domain organization and the phylogenetic segregation into a separate clade opened the possibility that the enzymes of the second clade represent a distinct subclass of GH5 endoglucanases.

### Example 10

### Purification and Properties of Other GH5 Endoglucanases

To determine the biochemical properties of the remaining GH5-containing cellulases, each polypeptide was purified to near homogeneity from Rosetta2™ (DE3) transformants as before either as the full length polypeptide (Cel5B, Cel5E, Cel5F) or the active catalytic domain (Cel5D′, Cel5G′, Cel5J′). The activity of purified Cel5B, ‘Cel5D, Cel5E and Cel5F were typical of classical endoglucanases. The enzymes had pH optima near 6.5, did not require salts and functioned up to 50° C. (Table S5).

TABLE S5
pH optimum

| Cellulase | MES pH = 5.0 | PIPES pH = 6.5 | TRIS pH = 8.0 |
|---|---|---|---|
| Cel5B | 100% | 92% | 95% |
| Cel5F | 54% | 100% | 63% |
| Cel5D | N/T | 100% | 84% |
| Cel5G | 76% | 100% | 71% |
| Cel5H^{a} | 21% | 100% | 23% |

^{a}Cel5H contained 0.04% Urea

In contrast to fungal cellulases (Kumar et al. (2008) Bioconversion of lignocellulosic biomass: biochemical and molecular perspectives. J Ind Microbiol Biot 35:377-391), Cel5B, Cel5D, Cell5E, Cel5F, and Cel5G retained at least 60% of the optimal activity at pH 8.0. As before, the activity was inversely proportional to the crystallinity of the cellulose substrate with the highest activity observed on CMC (Table 3).

TABLE 3
Specific Activities of the S. degradans GH5 cellulases

| | Specific activity on the indicated substrate^{a,b} | | | |
|---|---|---|---|---|
| | (μmol reducing sugar × minute⁻¹ × mg protein⁻¹) | | | |
| Cellulase^{c} | | Swollen | | |
| (kDa) | CMC^{d} | Cellulose | Filter Paper^{d} | Avicel ™^{d} |
| Cel5B (62) | 2.23 ± 16% | 1.53 × 10⁻² ± 4% | 1.84 × 10⁻³ ± 4% | 1.44 × 10⁻³ ± 8% |
| Cel5D (40) | 8.59 × 10⁻⁴ ± 26% | 1.13 × 10⁻⁴ ± 5% | 2.68 × 10⁻⁵ ± 28% | N/D^{e} |
| Cel5E (40) | 4.82 × 10⁻² ± 43% | 5.96 × 10⁻³ ± 8% | 4.01 × 10⁻⁶ ± 19% | N/D^{e} |
| Cel5F (42) | 0.37 ± 21% | 4.27 × 10⁻² ± 4% | 6.60 × 10⁻⁴ ± 7% | 6.26 × 10⁻⁴ ± 12% |
| Cel5G (42) | 4.62 ± 13% | 5.65 × 10⁻² ± 2% | 5.12 × 10⁻³ ± 4% | 4.88 × 10⁻³ ± 9% |
| Cel5H (72) | 9.61 ± 23% | 7.80 × 10⁻² ± 8% | 3.29 × 10⁻² ± 5% | 2.18 × 10⁻² ± 7% |
| Cel5J (38) | 6.97 ± 8% | 4.86 × 10⁻² ± 2% | 6.67 × 10⁻³ ± 6% | 2.18 × 10⁻³ ± 2% |

^{a}Reducing sugar released from CMC and swollen cellulose was measured using a DNS assay relative to a cellobiose standard curve whereas glucose released from filter paper and Avicel ™ was measured using the glucose oxidase method
^{b}milligrams of protein measured using BSA as the reference
^{c}The size of the largest polypeptide in a barley-β-glucan zymogram (kDa)
^{d}The approximate degree of crystallinity for each substrate (% crystallinity, 14): Carboxymethyl cellulose (CMC; soluble), PASC (10-20%), filter paper (50%), and Avicel ™ *(70%).
^{e}Activity not detected in a 15 h digestion period

Cel5B, Cel5D, and Cel5F exhibited product distributions typical of classical endoglucanases with ratios of soluble to insoluble products similar to that of other endoglucanases. The processivity ratios for Cel5G′ and Cel5J′, however, were all found to be significantly greater than 2 and each degraded filter paper, Avicel™, and pNP-cellobioside like Cel5H. Due to the presence of two distinct GH5 domains in Cel5A, processivity of the GH5 domains of this enzyme were not tested. The processivity of the other enzymes of clade 2 suggests that all of the GH5 domains of clade 2 are associated with processive activity. Interestingly, the activity of these processive endoglucanses was not dependent upon the activity of classic endoglucanases. For example, Cel5H was not synergistic with Cel5D, Cel5F, and Cel5G at three different molar ratios (1:4, 1:1, and 4:1), indicating that the enzyme does not recognize the free nonreducing ends of cellulose polymers like cellobiohydrolases (Table S6).

TABLE S6
Synergy within the S. degradans system

| | | Yield of Glucose (μg)^{a} | | | |
|---|---|---|---|---|---|
| | Nanomoles | Independent | Theoretical | Actual | |
| Cellulase | Cellulase | Yield^{b} | Yield^{c} | Yield | DoS^{d} |
| Cel5D | 1 | 0.26 ± 0.15^{e} | | | |
| Cel5F | 1 | 1.64 ± 0.045 | | | |
| | 0.5 | 1.23 ± 0.081 | | | |
| Cel5G | 0.8 | 6.17 ± 0.33 | | | |
| | 0.5 | 4.92 ± 0.79 | | | |
| | 0.2 | 2.64 ± 0.09 | | | |
| Cel5H | 0.1 | 20.4 ± 0.71^{e} | | | |
| | 0.8 | 18.7 ± 0.44 | | | |
| | 0.5 | 15.8 ± 2.27 | | | |
| | 0.2 | 11.1 ± 3.9 | | | |
| Cel5D + Cel5H | 1 + 0.1 (10:1) | | 20.7 | 12.2 ± 0.78 | 0.59 |
| Cel5F + Cel5H | 1 + 0.2 (5:1) | | 12.7 | 12.4 ± 0.44 | 0.98 |
| Cel5G + Cel5H | 0.8 + 0.2 (4:1) | | 17.3 | 12.0 ± 0.62 | 0.69 |
| | 0.5 + 0.5 (1:1) | | 20.7 | 16.9 ± 0.54 | 0.82 |
| | 0.2 + 0.8 (1:4) | | 21.3 | 19.3 ± 0.73 | 0.91 |
| Cel5G + Cel5F | 0.5 + 0.5 (1:1) | | 6.2 | 6.09 ± 0.27 | 0.98 |

^{a}The yield of glucose is measured from a two hour filter paper assay as described
^{b}The independent yield was measured after two hours as described for the filter paper assay
^{c}The sum of the μg glucose produced for both the S. degradans cellulases independently
^{d}DoS: The degree of synergy: The actual yield divided by the theoretical yield
^{e}Yield after 15 hours due to low activity of Cel5D

### Example 11

### Bacterial Growth Media and Conditions

Saccharophagus degradans 2-40^{T} (ATCC 43961) was grown in minimal medium containing (per liter) 2.3% Instant Ocean (Aquarium Systems, Mentor, Ohio), 0.05% Yeast Extract, 0.5% (w/v) ammonium chloride, and 16.7 mM Tris-HCl, pH 8.6 supplemented by 0.2% carbon source using standard protocols. Escherichia coli, DH5a (Invitrogen, Frederick, Md.) and Rosetta2™ (DE3) (Novagen, Madison, Wis.) strains were grown at 37° C. in Luria-Bertani (LB) broth or agar supplemented with the appropriate antibiotics. Antibiotics were added to media at the indicated concentrations (in μg/ml): chloramphenicol, 30; and kanamycin (Kan), 50.

### Example 12

### Bioinformatic Analyses

Similarities to the S. degradans sequences were based on local alignments obtained through the BLAST program. (Altschul et al. (1997) Gapped BLAST and PSI-BLAST: a new generation of protein database search programs. Nucleic Acids Res 25:3389-3402). Domain architectures were ascertained using SMART. (Schultz et al. (1998) SMART, a simple modular architecture research tool: Identification of signaling domains. Proc Natl Acad Sci USA 95:5857-5864). Multiple sequence alignments were created using clustalX 1.81, and manually adjusted where appropriate. (Thompson et al (1997) The CLUSTAL_X windows interface: flexible strategies for multiple sequence alignment aided by quality analysis tools. Nucleic Acids Res 25:4876-4882). The neighbor-joining algorithm was executed via the ClustalX 1.81 using the “Exclude Positions with Gaps” and “Correct for Multiple Substitutions” options. Statistical support for tree topology was provided by 1000 bootstrap trials.

### Example 13

### Molecular Cloning of S. Degradans GH5 Cellulases

S. degradans genomic DNA was isolated by using a commercial genomic DNA purification kit (Promega, Madison, Wis.). Sequences of annotated GH5 cellulases were extracted from the S. degradans genome sequence (http://maple.lsd.ornl.gov) and tailed primers were designed to amplify each individual GH5 cellulase by PCR (Table S2). Amplified fragments were ligated into pET28b (Novagen) as a BamH1-EcoR1 (Table S2) or BamH1-XhoI fragments (Table S2) to create in-frame amino and carboxy terminal 6×-His fusions and transformed into E coli DH5α. After confirmation of the correct construct in Kan^{R} transformants by nested PCR and/or sequencing, the resulting plasmid constructs were isolated and transformed into E. coli Rosetta2™ (DE3) for expression.

### Example 14

### Purification of Cellulases

Twenty ml of overnight Rosetta2™ (DE3)(pHZ-Cel) culture were inoculated into 500 ml LB broth and grown at 37° C. for 2 hours. Expression of cloned genes was induced by the addition of IPTG to a final concentration of 0.2 mM when OD₆₀₀=0.6. The culture was then incubated overnight at 15° C. with mild shaking. Cells from induced cultures were harvested and resuspended in 50 mM sodium phosphate, 300 mM sodium chloride, 10 mM imidizole, pH 8.0 and 0.5 mM Phenyl methyl sulfonyl fluoride. Lysozyme was added to a concentration of 1 mg/ml and the cell suspension incubated on ice for 30 minutes. Lysis of cells was completed using five 30s cycles in a Bead Beater™ (Biospec Products). The lysate was clarified by centrifugation at 10,000 RPM for 20 minutes. The expressed proteins in cleared lysates were purified using chelated Ni-NTA (Nitriltriacetic acid) chromatography according to the manufacturer's recommendations (Qiagen, 2003). The protein concentration was determined by the Pierce microBCA protein assay reagent kit (23235) using bovine serium albinum (BSA) as the standard.

### Example 15

### Zymograms

An adaptation of the procedures of Taylor et al was used to prepare zymograms. (Taylor et al (2006) Complete cellulase system in the marine bacterium Saccharophagus degradans strain 2-40(T). J Bacteriol 188:3849-3861). Samples and gels were prepared as in standard sodium dodecyl sulfate polyacrylamide gel electrophoresis (SDS-PAGE) with the indicated substrate incorporated directly into the resolving gel at a final concentration of 0.1% (w/v) for barley β-glucan (medium viscosity; Megazyme) or HE-cellulose. After electrophoretic fractionation of the proteins, gels were washed twice in distilled water and incubated in 30 ml of refolding buffer (20 mM PIPES (piperazine-N,N′-bis(2-ethanesulfonic acid)) buffer (pH 6.8), 2.5% Triton X-100, 2 mM dithiothreitol, 2.5 mM CaCl₂) for 1 hour at 20° C. and then held overnight in fresh refolding buffer at 4° C. The gels were washed twice in 20 mM PIPES buffer pH 6.8 and incubated for 12 h at 37° C. in fresh PIPES buffer. Residual substrate was visualized by staining with 0.25% Congo red.

### Example 16

### Enzyme Assays

Most assays were performed at pH 6.5 and 50° C. in a reaction mixture containing 1% Instant Ocean™ 20 mM PIPES buffer, and 0.01-1.0 nmol purified enzyme for soluble substrates and 0.1-2.0 nmol purified enzyme for insoluble substrates. The DNS (Dinitrosalicylic acid) assay was used to detect product formation unless indicated otherwise (32). All assays were performed in triplicate and reported as the mean with the percent error. For alternative pH conditions, 20 mM MES (2-(N-morpholino)ethanesulfonic acid) for pH 3-6, 20 mM PIPES (1,4-piperazinediethanesulfonic acid) for pH 6-7 or 20 mM Tris (2-Amino-2-(hydroxymethyl)propane-1,3-diol) for pH 7-8.5 were employed. The ionic strength of the reaction mixture was adjusted by the addition of 0-10% (w/v) Instant Ocean™.

The CMC assay was performed with 1% substrate in a total volume of 0.4 ml for a 15 min reaction time. Phosphoric acid swollen cellulose (PASC) was prepared as described Zhang et al. (2006). Biomacromolecules 7:644-648. The PASC (1 mg) assay was performed in a total volume of 0.15 ml for 30 minutes. Avicel™ (1 mg), or Whatman #1 filter paper (3 mg) was assayed in a total volume of 0.15 ml for 2 hours. The reaction was stopped by incubation for 3 minutes at 95° C. and the substrate separated by centrifugation at 10,000 rpm. The products of the insoluble substrates were digested with 0.45 nanomoles of the β-glucosidase S. degradans Bgl1A in a total volume of 0.2 ml at 50° C. for 1 hour. After 1 hour at 50° C., the β-glucosidase was inactivated by incubation for 3 minutes at 95° C. Glucose oxidase (Sigma GAGO-20) was added to a volume of 0.4 ml and incubated at 37° C. for 30 minutes. 0.4 ml of 12 N sulfuric acid was added and the glucose concentration was measured at OD₅₄₀ in comparison with a glucose standard curve. (Park et al. (2002) Molecular cloning and characterization of a unique β-glucosidase from Vibrio cholerae. J Biol Chem 277:29555-29560). Release of cellobiose was calculated at 50% the rate of glucose accumulation.

pNP-cellobioside activity was measured using 0.1 ml of 125 mM pNP-cellobioside in a total volume of 0.4 ml at 50° C. for 15 minutes. After incubation, the OD₄₀₀ was used to calculate the rate of nitrophenol release.

Total digestibility of cellulose was evaluated using 20 mg cotton linters (Sigma 435236) in a final volume of 1.0 ml in PIPES assay buffer with 2.0 nmols of the indicated enzyme and 1.25 nmols of β-glucosidase (S. degradans Bgl1A). At each time point 0.1 ml was removed and released sugar determined using the glucose oxidase assay as described above. Accellerase 1000 (Genecor Corp). was used to determine overall substrate accessibility.

### Example 17

### Assessment of Synergy

Evaluation of synergy between enzymes employed the filter paper assay as described above. T. fusca Cel9B and T. fusca Cel6B were used as reference enzymes of known activity (gifts of Professor David Wilson, Cornell University). Each assay utilized 1.0 nmol of S. degradans and 0.1 nmol the indicated T. fusca enzyme. Reaction conditions were as described above for the filter paper assay.

### Example 18

### Viscosity Measurements

Viscosity was monitored using a cross-arm viscometer (ASTM D455 and D2170). T. fusca Cel6B (0.01 nmol), S. degradans Cel5H (0.01 nmol) or T. fusca Cel9B (0.001 nmol) was added to 2.0 ml 1% CMC in assay buffer. The viscosity of the reaction mixture was measured periodically between 0.5-20 minutes.

### Example 19

### Thin Layer Chromatography

One μl samples were spotted onto Fisher (5729-6) silica gel 60 plates and air dried. Chromatograms were developed using nitromethane, 1-propanol, and water (2:5:1.5) (v:v:v) (35). Two ascents of the solvent were used to ensure high resolution. The plate was dipped in 5% (v/v) sulfuric acid in methanol and heated to 140° C. for 5 minutes to visualize resolved products.

### Example 20

### Processivity

The processivity was evaluated by the filter paper assay as described by Zhang et al. (2000). Eur J Biochem 267:3101-3115. The reducing sugar in the soluble fractions was measured as described above and reported in μmols of cellobiose. The insoluble reducing sugar was determined using a modified 2,2′-bicinchoninate (BCA) assay as described by Doner and Irwin (1992). Anal Biochem 202:50-53. At the end of the assay period, the filter paper was washed with 6M guanidine HCL to remove any bound protein. The filter paper disc was then washed 4 times with assay buffer and water. (Zhang et al. (2000), Eur J Biochem 267:3101-3115). The retained reducing sugar was measured using the Pierce microBCA reagent kit using glucose standards. The processivity was determined using 0.1-1 nmols S. degradans cellulases and 0.1 nmols of T. fusca Cel6A and T. fusca Cel6B.

### Example 21

### Expression of S. Degradans Cel5G, Cel5H, and Bgl1A in S. Degradans

Unless otherwise indicated, the recombinant DNA techniques utilized in the present example are standard procedures, well known to those skilled in the art. Such techniques are described and explained throughout the literature in sources such as, J. Perbal, A Practical Guide to Molecular Cloning, John Wiley and Sons (1984); J. Sambrook et al., Molecular Cloning: A Laboratory Manual, Cold Spring Harbor Laboratory Press (1989); T. A. Brown (editor), Essential Molecular Biology: A Practical Approach, Volumes 1 and 2, IRL Press (1991); D. M. Glover and B. D. Hames (editors), DNA Cloning: A Practical Approach, Volumes 1-4, IRL Press (1995 and 1996); and F. M. Ausubel et al. (Editors), Current Protocols in Molecular Biology, Greene Pub. Associates and Wiley-Interscience (1988, including all updates until present). These publications are incorporated herein by reference.

Isolation of Genomic DNA. Genomic DNA may be isolated using any method known in the art. Briefly, S. degradans was grown in a growth medium, as described herein elsewhere. Genomic DNA was isolated from the cells using standard techniques.

PCR was performed on a standard PCR machine under standard conditions. Briefly, PCR reactions contained 10 pMol of forward and reverse primers, 1 μl of 10 mM DNTPs, 1.5 μl of 100 mM MgCl₂, and 1 μl Proof Pro® Pfu Polymerase in a 501 reaction with 0.5 μl of S. degradans genomic DNA as the template. PCRs conditions used standard parameters for tailed primers and Pfu DNA polymerase. PCR products were cleaned up with the QIAGEN QIAquick PCR Cleanup kit and viewed in 0.8% agarose gels.

The primers used for amplification were as follows:

Zym5F Bam CCCGCGGATCCTGCTAAGCCGGAGGAAACC
Zym5R Kpn CCGGGGTACCATAAGCACGAACTTTAACGT
Zym6F Xba GCTCTAGAAAGGCGCATTGCGCCAACTAT
Zym6R Xma TCCCCCCGGGACTGACTAGAACCCACCATCT T
Zym9F Bam CCCGGATCCAATTCTTAGCGGTGGCCAGCAA
Zym9R Xho CCCGCTCGAGCCAGCTACCAAATTGCAGGGTGT
pET F Xba CTAGTCTAGAAACTTTAAGAAGGAGATATACCAT
pET R Sma TCCCCCCGGGATCTCAGTGGTGGTGGTGGTGGTG

The gene for Zym9 was first cloned into the cloning vector pET28b. The genes were then amplified from the pET clones using the primers pETF and pETR and ligated into pDSK600 before transformation into S. degradans.

Following cleanup and confirmation of size, PCR products are ligated into the pDSK600 plasmid. The pDSK600 plasmid is a broad host plasmid and contains a SpR marker and robust 3× placUV promotor.

Competent S. degradans cells are then prepared. Briefly, a culture of S. degradans was grown to OD600 0.6-1.0. The culture is placed on ice for 15 min and then centrifuge for 10 min at 4° C. Resuspend by 0.7 mol/L sucrose, centrifuge at 4° C., 8000 rpm for 10 min. Repeat above procedure for 2 times.

The pDSK600/Zym5, pDSK600/Zym6, pDSK600/Zym8, and pDSK600/Zym9 expression vectors were then electroporated into the S. degradans competent cell. Briefly the electroporation was performed using 0.2 mm cuvette at 1500V; 3 μl pDSK600 for 50 μl cells. The cells were then transferred to 1 ml fresh medium and subculture for 3-4 h. The cells where then spread on plates with antibiotics.

Zymogram of Zym5 and Zym8 grown on Avicel. Activity assays were then performed. Briefly, a culture of the Zym and wild type S. degradans strains were prepared. The cells were then lysed to make the enzyme samples. Next, 100 ul enzyme was incubated with 100 ul CMC and 200 ul buffer for 15 min. Then 1 ml DNS reagents was added and boiled for 15 min. Absorbance at OD600 was measured.

TABLE
Overexpression of Endoglucanases

| | | | Activities | Activities |
|---|---|---|---|---|
| | Enzyme Over | | compared with | compared with |
| Strain | Expressed | Secretion | WT (Avicel) | WT (Glucose) |
| Zym5 | Cel5G | + | 3.5 | 108 |
| Zym7 | Cel5G | (−) | 1.7 | 291 |
| Zym8 | Cel5J | (−) | 0.9 | 77 |
| Zym9 | Cel5H | + | 2.6 | 691 |

(−) Deleted secretion signal on polypeptide

TABLE
Overexpression β-Glucosidase

| | | | Activities | Activities |
|---|---|---|---|---|
| | Enzyme Over | | compared with | compared with |
| Strain | Expressed | Secretion | WT (Avicel) * | WT (Glucose) * |
| Zym6 | Bgl1A | (−) | 29 | 105 |

(−) Deleted secretion signal on polypeptide
* Degradation of cellobiose

### Example 22

### Zym Strains Evaluation

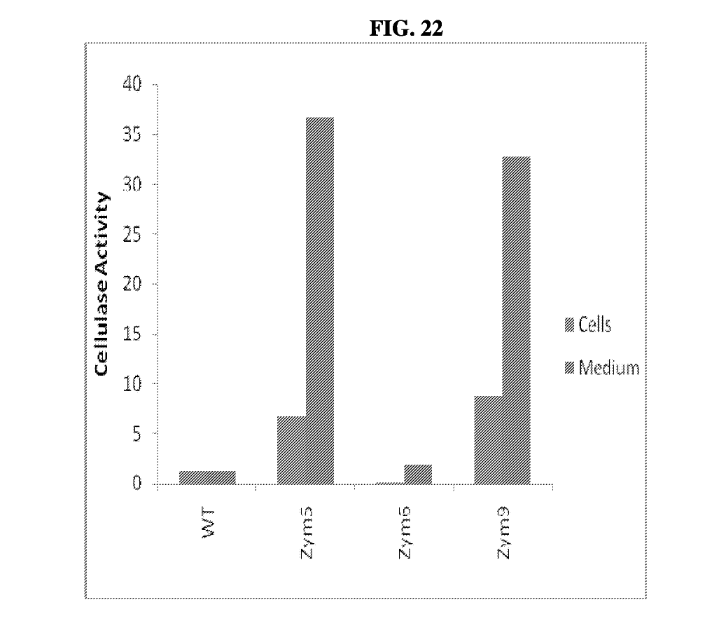

Protein preps. Frozen cultures of Zym5, Zym6, Zym9, and the wild-type S. degradans 2-40 were used to inoculate 5 mL of the 2-40 medium supplemented with 0.2% glucose. The strains were gown overnight at 29° C. on a orbital shaker. Densities of the bacterial cultures were measured spectrophotometrically at 600 nm. Densities of these cultures were normalized (adjusted to OD₆₀₀ 0.793). Two milliliters of the normalized cultures were used to inoculate 50 mL of the 2-40 medium containing 1% Avicel. The Avicel cultures were incubated for 27 hrs at 29° C. on a orbital shaker at 200 rpm. Densities of the cultures were measured again. Since Avicel is insoluble in water, the OD₆₀₀ readings were taken after 30-min settling period. The cells and the medium were separated by centrifugation at 7,000×g for 15 minutes. The OD₆₀₀ readings were used to normalize protein preps. The cell protein preps were obtained by osmotic lysis of the S. degradans cells in deionized water. Amount of the water added to the cells, and dilution factor for the medium protein preps were calculated based on the OD₆₀₀ readings. See FIG. 22.

For evaluation of the cellulase activity of the cell and the media protein preps, AZCL-HE-Cellulose was used (Megazyme). AZCL-HE-Cellulose is a chromogenic water-insoluble substrate for cellulases. A typical reaction mixture used in this experiment contained 2 mg of the substrate, 900 μL of 50 mM citrate buffer, pH 6.0, and 100 μL of the protein prep. Upon degradation of this substrate by the enzymes, a blue dye is released into the reaction mixture. Amount of the dye released per time period is proportional to the cellulolytic activity in the reaction mixture. The reaction mixtures were incubated at 50 C for 3 hrs, and absorbance of the supernatant was measured at 590 nm. The OD₅₉₀ readings were used to produce the data shown in FIG. 25. Comparison of the cellulase preps from different strains was performed on “per cell” basis. Total cellulase activity of the protein preps from the medium and the cells of the wild-type S. degradans was equal.

### EQUIVALENTS AND INCORPORATION BY REFERENCE

Those skilled in the art will recognize, or be able to ascertain using no more than routine experimentation, numerous equivalents to the specific polypeptides, nucleic acids, methods, assays and reagents described herein. Such equivalents are considered to be within the scope of this invention and are covered by the following claims.

The instant application includes numerous citations to learned texts, published articles and patent applications as well as issued U.S. and foreign patents. The entire contents of all of these citations are hereby incorporated by reference herein.

### REFERENCES CITED

1 Altschul, S. F. et al (1997) Gapped BLAST and PSI-BLAST: a new generation of protein database search programs. Nucleic Acids Res 25:3389-3402.
2 Andrykovitch, G. and I. Marx (1988) “Isolation of a new polysaccharide-digesting bacterium from a salt marsh.” Applied and Environmental Microbiology 54: 3-4.
3 Bayer, E. A., Chanzy, H., Lamed, R., and Shoham, Y. (1998) Cellulose, cellulases and cellulosomes. Curr Opin Struc Biol 8:548-557.
4 Beguin, P. and J. P. Aubert (1994) “The biological degradation of cellulose.” FEMS Microbiol Rev 13(1): 25-58.
5 Boraston, A. B., Bolam, D. N., Gilbert, H. J., and Davies, G. J. (2004) Carbohydrate-binding modules: fine-tuning polysaccharide recognition. Biochem J 382:769-781.
6 Breyer, W. A. and Matthews, B. W. (2001) A structural basis for processivity. Protein Sci 10:1699-1711.
7 Chakravorty, D. (1998). Cell Biology of Alginic Acid degradation by Marine Bacterium 2-40. College Park, University of Maryland.
8 Cohen, R., Suzuki, M. R., and Hammel, K. E. (2005) Processive endoglucanase active in crystalline cellulose hydrolysis by the brown rot basidiomycete Gloeophyllum trabeum. Appl Environ Microbiol 71:2412-2417.
9 Coutinho, P. M. and B. Henrissat (1999) Carbohydrate-active enzyme server. Accessed Jan. 21, 2004.
10 Coutinho, P. M. and B. Henrissat (1999) The modular structure of cellulases and other carbohydrate-active enzymes: an integrated database approach. Genetics, biochemistry and ecology of cellulose degradation. T. Kimura. Tokyo, Uni Publishers Co: 15-23. Distel, D. L., W. Morrill, et al. (2002) “Teredinibacter turnerae gen. nov., sp. nov., a dinitrogen-fixing, cellulolytic, endosymbiotic gamma-proteobacterium isolated from the gills of wood-boring mollusks (Bivalvia: Teredimidae).” Int J Syst Evol Microbiol 52(6): 2261-2269.
11 Doi, R. H. and Kosugi, A. (2004) Cellulosomes: Plant-cell-wall-degrading enzyme complexes. Nat Rev Microbiol 2:541-551.
12 Doner, L. W. and Irwin, P. L. (1992) Assay of reducing end-groups in oligosaccharide homologs with 2,2′-bicinchoninate. Anal Biochem 202:50-53.
13 Ducros, V. et al (1995) Crystal-structure of the catalytic domain of a bacterial cellulase belonging to family-5. Structure 3:939-949.
14 Ensor, L., S. K. Stotz, et al. (1999) “Expression of multiple insoluble complex polysaccharide degrading enzyme systems by a marine bacterium.” J Ind Microbiol Biotechnol 23: 123-126.
15 Ghose, T. K. (1987) Measurement of cellulase activities. Pure Appl Chem 59:257-268.
16 Gilad, R. et al (2003) Cell, a noncellulosomal family 9 enzyme from Clostridium thermocellum, is a processive endoglucanase that degrades crystalline cellulose. J Bacteriol 185:391-398.
17 Gonzalez, J. and R. M. Weiner (2000) “Phylogenetic characterization of marine bacterium strain 2-40, a degrader of complex polysaccharides.” International journal of systematic evolution microbiology 50: 831-834.
18 Henrissat, B. and A. Bairoch (1993) “New families in the classification of glycosyl hydrolases based on amino acid sequence similarities.” Biochem J 293 (Pt 3): 781-8.
19 Henrissat, B., T. T. Teeri, et al. (1998) “A scheme for designating enzymes that hydrolyse the polysaccharides in the cell walls of plants.” FEBS Lett 425(2): 352-4.
20 Himmel, M. E. (2007) Biomass recalcitrance: engineering plants and enzymes for biofuels production. Science 316:982-982.
21 Horn, S. J. et al (2006) Costs and benefits of processivity in enzymatic degradation of recalcitrant polysaccharides. Proc Natl Acad Sci USA 103:18089-18094.
22 Howard, M. B. et al (2004) Identification and analysis of polyserine linker domains in prokaryotic proteins with emphasis on the marine bacterium Microbulbifer degradans. Protein Sci 13:1422-1425.
23 Irwin, D. C., Spezio, M., Walker, L. P., and Wilson, D. B. (1993) Activity studies of 8 purified cellulases-specificity, synergism, and binding domain effects. Biotechnol and Bioeng 42:1002-1013.
24 Jeoh, T., Wilson, D. B., and Walker, L. P. (2006) Effect of cellulase mole fraction and cellulose recalcitrance on synergism in cellulose hydrolysis and binding. Biotechnol Progr 22:270-277.
25 Jonsson, A. P., Y. Aissouni, et al. (2001) “Recovery of gel-separated proteins for in-solution digestion and mass spectrometry.” Anal Chem 73(22): 5370-7.
26 Kang, M. S. et al (2007) Effect of Leuconastoc spp. on the formation of Streptococcus mutans biorilm. J Microbiol 45:291-296.
27 Kelley, S. K., V. Coyne, et al. (1990) “Identification of a tyrosinase from a periphytic marine bacterium.” FEMS Microbiol Lett 67: 275-280.
28 Kosugi, A., K. Murashima, et al. (2002) “Characterization of two noncellulosomal subunits, ArfA and BgaA, from Clostridium cellulovorans that cooperate with the cellulosome in plant cell wall degradation.” J Bacteriol 184(24): 6859-65.
29 Kumar, R., Singh, S., and Singh, O. V. (2008) Bioconversion of lignocellulosic biomass: biochemical and molecular perspectives. J Ind Microbiol Biot 35:377-391.
30 Laemmli, U. K. (1970). “Cleavage of structural proteins during the assembly of the head of the bacteriophage T4.” Nature 277: 680-685.
31 Li, Y. C., Irwin, D. C., and Wilson, D. B. (2007) Processivity, substrate binding, and mechanism of cellulose hydrolysis by Thermobifida fusca Cel9A. Appl Environ Microbiol 73:3165-3172.
32 Ljungdahl, L. G. and K. E. Eriksson (1985) Ecology of Microbial Cellulose Degradation. Advances in Microbial Ecology. New York, Plenum Press. 8: 237-299.
33 Lo Leggio, L. and Larsen, S. (2002) The 1.62 angstrom structure of Thermoascus aurantiacus endoglucanase: completing the structural picture of subfamilies in glycoside hydrolase family 5. FEBS Lett 523:103-108.
34 Lou, J., K. Dawson, et al. (1996) “Role of phosphorolytic cleavage in cellobiose and cellodextrin metabolism by the ruminal bacterium Prevotella ruminicola.” Appl. Environ. Microbiol. 62(5): 1770-1773.
35 Lynd, L. R., P. J. Weimer, et al. (2002) “Microbial cellulose utilization: fundamentals and biotechnology.” Microbiol Mol Biol Rev 66(3): 506-77, table of contents.
36 Martinez, D. et al (2008) Genome sequencing and analysis of the biomass-degrading fungus Trichoderma reesei (syn. Hypocrea jecorina). Nat Biotechnol 26:1193-1193.
37 Park, J. K., Wang, L. X., Patel, H. V., and Roseman, S. (2002) Molecular cloning and characterization of a unique β-glucosidase from Vibrio cholerae. J Biol Chem 277:29555-29560.
38 Qi, M., Jun, H. S., and Forsberg, C. W. (2007) Characterization and synergistic interactions of Fibrobacter succinogenes glycoside hydrolases. Appl Environ Microbiol 73:6098-6105.
39 Qi, M., Jun, H. S., and Forsberg, C. W. (2008) Cel9D, an atypical 1,4-1′-D-glucan glucohydrolase from Fibrobacter succinogenes: characteristics, catalytic residues, and synergistic interactions with other cellulases. J Bacteriol 190:1976-1984.
40 Rubin, E. M. (2008) Genomics of cellulosic biofuels. Nature 454:841-845.
41 Sakon, J., Irwin, D., Wilson, D. B., and Karplus, P. A. (1997) Structure and mechanism of endo/exocellulase E4 from Thermomonospora fusca. Nat Struct Biol 4:810-818.
42 Schultz, J., Milpetz, F., Bork, P., and Ponting, C. P. (1998) SMART, a simple modular architecture research tool: Identification of signaling domains. Proc Natl Acad Sci USA 95:5857-5864.
43 Shevchenko, A., M. Wilm, et al. (1996) “Mass spectrometric sequencing of proteins silver-stained polyacrylamide gels.” Anal Chem 68(5): 850-8.
44 Smith, R. D., J. A. Loo, et al. (1990) “New developments in biochemical mass spectrometry: electrospray ionization.” Anal Chem 62(9): 882-99.
45 Stotz, S. K. (1994). An agarase system from a periphytic prokaryote. College Park, University of Maryland.
46 Sumner, J. B. and E. B. Sisler (1944) “A simple method for blood sugar.” Archives of Biochemistry 4: 333-336.
47 Taylor, L. E. et al (2006) Complete cellulase system in the marine bacterium Saccharophagus degradans strain 2-40(T). J Bacteriol 188:3849-3861.
48 Thompson, J. D. et al (1997) The CLUSTAL_X windows interface: flexible strategies for multiple sequence alignment aided by quality analysis tools. Nucleic Acids Res 25:4876-4882.
49 Tomme, P., R. A. Warren, et al. (1995) “Cellulose hydrolysis by bacteria and fungi.” Adv Microb Physiol 37: 1-81.
50 Violot, S. et al (2005) Structure of a full length psychrophilic cellulase from Pseudoalteromonas haloplanktis revealed by x-ray diffraction and small angle x-ray scattering. J Mol Biol 348:1211-1224.
51 Warren, R. A. (1996) “Microbial hydrolysis of polysaccharides.” Annu Rev Microbiol 50: 183-212.
52 Weiner, R. M. et al (2008) Complete genome sequence of the complex carbohydrate-degrading marine bacterium, Saccharophagus degradans strain 2-40(T). PLOS Genet. 4:e100087
53 Whitehead, L. (1997). Complex Polysaccharide Degrading Enzyme Arrays Synthesized By a Marine Bacterium. College Park, University of Maryland.
54 Wilson, D. B. (2004) Studies of Thermobifida fusca plant cell wall degrading enzymes. Chem Rec 4:72-82.
55 Wilson, D. B. (2008) Three microbial strategies for plant cell wall degradation. Ann NY Acad Sci 1125:289-297.
56 Xie, G. et al (2007) Genome sequence of the cellulolytic gliding bacterium Cytophaga hutchinsonii. Appl Environ Microbiol 73:3536-3546.
57 Zhang, S., Barr, B. K., and Wilson, D. B. (2000) Effects of noncatalytic residue mutations on substrate specificity and ligand binding of Thermobifida fusca endocellulase Cel6A. Eur J Biochem 267:244-252.
58 Zhang, S., Irwin, D. C., and Wilson, D. B. (2000) Site-directed mutation of noncatalytic residues of Thermobifida fusca exocellulase Cel6B. Eur J Biochem 267:3101-3115.
59 Zhang, Y. H. P., Cui, J. B., Lynd, L. R., and Kuang, L. R. (2006) A transition from cellulose swelling to cellulose dissolution by o-phosphoric acid: Evidence from enzymatic hydrolysis and supramolecular structure. Biomacromolecules 7:644-648.
60 Zhang, Y. H. P., Himmel, M. E., and Mielenz, J. R. (2006) Outlook for cellulase improvement: Screening and selection strategies. Biotechnol Adv 24:452-481.

What is claimed is:

1. A genetically modified host cell, wherein said host cell is genetically modified with one or more isolated nucleic acids selected from the group consisting of:
a. a polynucleotide having a sequence of Cel5A, Cel5G, Cel5H, or Cel5J;
b. a polynucleotide encoding a polypeptide having a sequence of Cel5A, Cel5G, Cel5H, or Cel5J;
c. a polynucleotide having at least 95% sequence identity to a polynucleotide having a sequence of Cel5A, Cel5G, Cel5H, or Cel5J, wherein the polynucleotide encodes a polypeptide having processive endoglucanase activity;
d. a polynucleotide encoding a polypeptide having at least 95% sequence identity to a polypeptide having a sequence of that encodes any of Cel5A, Cel5G, Cel5H, or Cel5J, wherein the polynucleotide encodes a polypeptide having processive endoglucanase activity; and
e. a polynucleotide that hybridizes under stringent conditions to the complement of any of the polynucleotides of a) through d) above, wherein the polynucleotide encodes a polypeptide having processive endoglucanase activity.

2. The genetically modified host cell of claim 1, wherein the expression of the polynucleotide in the host cell results in increased yield of cellobiose in the presence of cellulosic material.

3. The genetically modified host cell of claim 1, wherein the expression of the polynucleotide in the host cell is at least 50-fold over control levels.

4. The genetically modified host cell of claim 1, wherein the host cell is Saccharophagus degradans.

5. A method of producing a genetically modified host cell containing an isolated nucleic acid encoding a polypeptide, wherein the method comprises the steps of transforming a host cell with an expression vector comprising the nucleic acid, wherein the nucleic acid comprises a polynucleotide selected from the group consisting of:
a. a polynucleotide having a sequence that encodes any of Cel5A, Cel5G, Cel5H, or Cel5J;
b. a polynucleotide encoding a polypeptide having a sequence of Cel5A, Cel5G, Cel5H, or Cel5J;
c. a polynucleotide having at least 95% sequence identity to a polynucleotide having a sequence as that encodes any of Cel5A, Cel5G, Cel5H, or Cel5J, wherein the polynucleotide encodes a polypeptide having processive endoglucanase activity;
d. a polynucleotide encoding a polypeptide having at least 95% sequence identity to a polypeptide having a sequence of Cel5A, Cel5G, Cel5H, or Cel5J, wherein the polynucleotide encodes a polypeptide having processive endoglucanase activity; and
e. a polynucleotide that hybridizes under stringent conditions to the complement of any of the polynucleotides of a) through d) above, wherein the polynucleotide encodes a polypeptide having processive endoglucanase activity.

6. The method of claim 5, wherein the nucleic acid is operably linked to one or more regulatory sequences.

7. The method of claim 5, wherein the regulatory sequence is a promoter.
